(19) United States
(12) Patent Application Publication
(10) Pub. No.: US 2012/0295893 A1
(43) Pub. Date: Nov. 22, 2012

(54) COMPOSITIONS AND METHODS OF COUNTERACTING RESIDUAL SEDATIVE EFFECTS OF SLEEP/ HYPNOTIC DRUGS

(75) Inventors: Nir Peled, Hod Hasharon (IL); David Solomon, Zichron Yaakov (IL); Amir Toren, Zichron Yaakov (IL)

(73) Assignee: COERULEUS LTD., Katzrin (IL)

(21) Appl. No.: 13/515,747

(22) PCT Filed: Dec. 14, 2010
(86) PCT No.: PCT/IL10/01062

§ 371 Date: Jun. 13, 2012

Related U.S. Application Data
(60) Provisional application No. 61/286,033, filed on Dec. 14, 2009.
(60) Provisional application No. 61/331,397, filed on May 5, 2010.

(51) Int. Cl. A61K 31/5517; A61P 11/00; A61P 25/32; A61P 43/00

(52) U.S. Cl. 514/220

(57) ABSTRACT

The present invention provides pharmaceutical compositions comprising a flumazenil, and methods of alleviating or counteracting residual effects (e.g. drowsiness) associated with the administration of sleep/hypnotic drugs or alleviating effects of alcohol intoxication, using self administration modes of delivery.

### FIELD OF THE INVENTION

The present invention provides pharmaceutical compositions comprising a flumazenil, and methods of alleviating or counteracting residual effects (e.g. drowsiness) associated with the administration of sleep/hypnotic drugs or alleviating effects of alcohol intoxication, using self administration modes of delivery.

### BACKGROUND OF THE INVENTION

The clinical disorder termed insomnia represents a subjective perception of dissatisfaction with the amount and/or quality of sleep, and is associated with complaints of non-restorative sleep and dysfunction of diurnal alertness, energy, cognitive function, behavior or emotional state, with a secondary decrease in quality of life. Although insomnia is a common problem, it is nevertheless under-diagnosed since only 5% of patients with insomnia specifically seek medical help, while 70% never inform a physician of their disorder. Insomnia is commonly treated with sleep drugs, however, many sleep drugs, in particular those with a longer half life, are associated with post arousal drowsiness in about 30% of individuals who use them. This undesirable effect is a major cause of the lack of patient compliance associated with many sleep drugs, which contributes to the cycle of insomnia and persistent patient discomfort.

Flumazenil is an imidazobenzodiazepine with high affinity to the GABA_{A}/benzodiazepine-receptor complex, the specific binding site of benzodiazepines. In addition, flumazenil is a competitive inhibitor of benzodiazepines, thus used to reverse benzodiazepine-induced sedation and anesthesia following therapeutic or diagnostic procedures (e.g. Goldfrank, L. R. (2002) Goldfrank's toxicologic emergencies. New York: McGraw-Hill). Flumazenil is also known to reverse the effect of non-benzodiazepine drugs, such as the imidazopyridine hypnotic zolpidem (e.g. Patat et al., Clin Pharmacol Ther. 1994 October; 56(4):430-6). Flumazenil was also shown effective in treating idiopathic hypersomnia by i.v. infusion for 2 days (Clark, http://www.emory.edu/EMORY_MAGAZINE/2008/winter/sleeping). In addition, flumazenil, in combination with hydroxyzine and gabapentin, was successful in the treatment of stimulant addiction (methamphetamine; Bond A J, 1998, CNS Drugs, 9(1): 41-57. doi:10.2165/00023210-199809010-00005.)

A liquid form of flumazenil for intravenous (i.v.) injection (Romazicon®) is approved for the reversal of the sedative effects of benzodiazepines. The current usage of flumazenil known in art is sporadic, i.e. on specific occasions as needed to treat medical conditions and not on a daily basis. In addition, the common usage of flumazenil is by a physician/health provider, and not in a self-administered manner.

A single intraoral administration of flumazenil has been disclosed to incompletely reverse deep sedation from incremental sublingual dosing of triazolam in a randomized controlled clinical trial of flumazenil as a rescue strategy (Hosaka et al., J Am Dent Assoc. 2009 May; 140(5):559-66).

U.S. Patent Application Publication Nos. 2005/0031688 and 2009/0041840 disclose a sleep regulating pharmaceutical formulation having two principal drugs having opposing actions to one another, incorporated into a unitary solid dosage form for oral administration. The outer area of the dosage form comprises a drowsiness promoting drug and the core comprises an arousal promoting/stimulant drug coated with an osmotic semi-permeable membrane. According to this disclosure, flumazenil is not suitable for incorporation into the formulation since it is not orally administrable and is not a stimulant.

PCT Patent Application Publication No. WO 2009/114740 discloses methods of treating GABA_{A} receptor mediated hypersomnia and excessive sleepiness associated with GABA_{A} receptor mediated hypersomnia, by administering to a subject in need thereof an effective amount of a GABA_{A} receptor antagonist inter alia flumazenil. According to the disclosure, the invention is directed to treatment of excessive sleepiness disorders in patients having one or more endogenous substances present, typically in excess, in their CSF that act as positive allosteric modulators of the GABA_{A} re ceptor. Also disclosed are tablets for sublingual administration comprising flumazenil, tablet triturate base (powdered sucrose and hydrous lactose monohydrate), unflavored tablet triturate excipient, flavoring agent and Stevia concentrate (Stevia powder extract, sodium benzoate and water), however, chronic (e.g. daily usage) and self-administered profile, are not disclosed

The U.S. Army Medical Research and Materiel Command has disclosed the results of a study investigating the sporadic (single day) usage of a liquid intravenous formulation of flumazenil administered sublingually for reversing soporific effects induced by zolpidem (Eddy et al., AFRL-RH-BR-TR-2009-0026; Public Affairs Case File No. 09-271, 15 Jun. 2009). This study was published after the conception of the present invention by the present inventors. According to the disclosure, sublingual administration of flumazenil i.v. formulation is only partially effective for nullifying the soporific effects of zolpidem, in particular since the effects of zolpidem were reversed for only one to two hours, and a second dose of flumazenil had no beneficial effect. Moreover, chronic (e.g. on a daily basis) usage of flumazenil was not taught or suggested.

U.S. Pat. No. 6,977,070 discloses a method of administering a pharmacologically active compound to provide transmucosal absorption of the compound through the oral mucosa, comprising spraying the oral mucosa with a buccal spray composition. According to the disclosure, the active compound may be a benzodiazepine antagonist, such as flumazenil. According to the disclosure, the preferred composition comprises in weight percent of the composition, 0.1 to 25% of the active compound; 10 to 97% of a polar solvent, and 2 to 10% of a C₃ to C₈ hydrocarbon propellant.

There remains an unmet need for pharmaceutical compositions effective in countering drug induced sedation, that are safe and suitable for single or repetitive use and particularly for self administration use.

### SUMMARY OF THE INVENTION

The present invention provides improved pharmaceutical compositions and methods of counteracting residual effects associated with the administration of sleep/hypnotic drugs, taken by subjects who are insomniacs (or are otherwise in need of sleep/hypnotic drugs), or administered to subjects during medical procedures which require sedation and/or anesthesia. In particular, the invention addresses the need for improved patients compliance in the methods directed to alleviate excessive sleepiness and drowsiness during waking hours when a sedated state is undesirable and counterproductive to the therapeutic goal of achieving a beneficial sleep-waking cycle, in particular in insomniac patients. The invention further addresses the need for improved patients compliance in the methods directed to alleviate excessive sleepiness and drowsiness caused by sleep drugs such as benzodiazepines in cases where anesthesia has been induced and/or maintained with such sleep drugs, where sedation has been produced with the sleep drugs for diagnostic or therapeutic procedures, or for the management of sleep drug overdose. It has unexpectedly been discovered that the formulations of the present invention, which are formulated for sublingual administration, are superior to conventional formulations given by intravenous route, as demonstrated in preclinical (animal) and clinical studies. The invention further provides methods for reversing, reducing or alleviating effects of alcohol intoxication, or improving performance after alcohol consumption using the pharmaceutical composition of the invention. The methods of the invention are particularly suitable for flumazenil self administration (e.g. at home) on a sporadic and/or chronic daily basis for the above mentioned indications. The methods of the invention may be also directed to administration by professional care takers, but do not necessarily require this kind of involvement.

The methods of the invention comprise administration of a GABA_{A} receptor antagonist, in particular flumazenil, generally using formulations which provide sublingual, transmucosal or transdermal delivery, and any other delivery suitable for self administration. While intravenous administration of flumazenil is known in the art for reversing GABA_{A} receptor-targeted sedation and anesthesia, the present invention provides a novel flumazenil composition formulated for sublingual administration, a new dosing regimen and new indications for flumazenil and related drugs, directed primarily at the prevention and treatment of the residual effects, e.g. the excessive drowsiness associated with the use of sleep drugs. The invention is of particular use in insomniac patients with a chronic usage of sleep drugs, but is also applicable to individuals using sleep drugs only on an occasional basis, for example due to short-term stress or air travel.

The invention is based in part on the unexpected discovery of flumazenil formulations that provide effective treatment for counteracting post awakening sleep drug-induced drowsiness when administered via transmucosal delivery. The invention provides beneficial clinical effects, beyond the expected duration, in a reliable and reproducible fashion, thereby contributes to improved patient compliance and to adequate use of sleep drugs. Moreover, the methods and formulations of the invention are intended for self-administration and/or for chronic usage (e.g. on a daily basis), and together with a suitable sleep drug, provide a therapeutic modality of insomnia with minimal side effects and with a safer post-awakening functioning.

The formulation of the invention is more effective for sublingual administration than the commercially available liquid formulation of flumazenil (Romazicon®) when the latter is given sublingually (Ro Mazicon® is marketed for i.v. administration. The advantageous effect of the formulation of the invention is exhibited by improved countering the excessive sleepiness induced by sleep drugs resulting in better performance. The formulation of the invention is advantageous over the commercially available liquid formulation of flumazenil for i.v. administration (Romazicon®). Sublingual administration of the commercial formulation was only partially effective for nullifying the sedative effects of the benzodiazepine hypnotic zolpidem. However the formulation of the invention when administered sublingually successfully counteracted the sedative effects of benzodiazepine hypnotics (diazepam) as well as of non-benzodiazepine hypnotics (Zolpidem™) in preclinical and clinical studies, as exemplified herein. The formulation of the invention was also shown to be advantageous over a non-formulated flumazenil USP solution which merely includes a solubilizing agent (Tween 80) and saline, as exemplified herein. Surprisingly, the strong countering effect exhibited by the formulation of the invention is maintained for at least 60 minutes after administration in humans and no rebound effect of the hypnotics was witnessed. In fact, the anti-sedating effect of the formulation of the invention is enhanced an hour after administration as compared with 20 minutes after administration. An additional advantage of the formulation of the invention is the relatively high concentration of flumazenil, which enables achieving an effective therapeutic dose of relatively small volumes thereby providing an improved convenience of use. Furthermore, the formulation of the invention contains a lower amount of non-active excipients as compared to the commercial formulation.

Without wishing to be bound by any particular theory or mechanism of action, it is hypothesized that the efficacy of the invention may be attributed to the ability of GABA_{A} receptor antagonists such as flumazenil to competitively inhibit binding of sleep/hypnotic drugs such as benzodiazepines, benzodiazepine analogs such as thienodiazepines and non-benzodiazepines to their target neurological receptors.

According to a first aspect, the present invention provides a liquid formulation for sublingual administration, the formulation comprising flumazenil as an active ingredient, a solubilizing agent selected from an alcohol, a glycol and a combination thereof, a buffering agent, and at least one agent selected from the group consisting of: a penetration enhancer, a surfactant and cyclodextrin. Each possibility represents a separate embodiment of the present invention.

According to one embodiment, the cyclodextrin is hydroxypropyl β-cyclodextrin (HPCD). The cyclodextrin is preferably formulated in a buffer having a pH from about 3 to about 6. In one particular embodiment, the cyclodextrin (e.g., HPCD) is formulated in a citric acid buffer having a pH of about 4.

The solubilizing agent is preferably an alcohol or a glycol, for example ethanol or propylene glycol, or a combination thereof. In one particular embodiment, the solubilizing agent is a combination of ethanol and propylene glycol. Each possibility represents a separate embodiment of the present invention.

According to yet another embodiment, the preservative is selected from the group consisting of benzyl alcohol, propylparaben, methylparaben and combinations thereof. In one embodiment, the preservative is benzyl alcohol. In another embodiment, the preservative is a combination of propylparaben and methylparaben. Each possibility represents a separate embodiment of the present invention.

According to yet another embodiment, the penetration enhancer is menthol. It has been surprisingly found that the inclusion of penetration enhancers, e.g. menthol, significantly improves the performance of the formulations in the utilities described herein, as opposed to conventional formulations which lack such excipient. In a preferred embodiment, menthol also improves the flavor of the formulation.

According to yet another embodiment, the buffering agent is selected from the group consisting of: citric buffer, sodium chloride and combination thereof.

According to yet another embodiment, the surfactant is a cationic surfactant. According to yet another embodiment, the surfactant is benzalkonium chloride.

According to yet another embodiment, the formulation comprises a plurality of agents selected from the group consisting of: a penetration enhancer, a preservative, a surfactant, a cyclodextrin, and a solubilizing agent.

According to yet another embodiment, the flumazenil formulation of the invention is provided in a form selected from the group consisting of: sublingual dosage form, transdermal dosage form, subdermal dosage form, aerosol form (for inhalation) and transmucosal dosage form.

According to yet another embodiment, the flumazenil formulation of the invention is provided in a form for transmucosal delivery to a mucosal surface of the oral cavity or nasal cavity. According to yet another embodiment, the mucosal surface is selected from the group consisting of the buccal mucosa, the sublingual mucosa, the gingival mucosa, the palatal mucosa, the labial mucosa, the sinusoidal mucosa, the nasal mucosa, and a combination thereof.

According to yet another embodiment, the flumazenil formulation of the invention is suitable for chronic (repeated) usage, such that it is administered a few times a day, daily, several times a week, weekly, and the like.

According to yet another embodiment, the formulation is in a liquid form for sublingual administration.

According to yet another embodiment, the formulation is provided in the form of a sublingual spray, e.g., a sublingual spray device (FLUMUP SL) vial, pump and actuator.

According to another aspect, the present invention provides a liquid formulation for sublingual administration, the formulation comprising flumazenil as an active ingredient, and ethanol, propylene glycol, HPCD in citric buffer 10 mM pH 4.0 and menthol, as inactive excipients. In one embodiment, the formulation further comprises benzyl alcohol or propylparaben/methylparaben as a preservative. Preferably the formulation is provided in the form of a sublingual spray, e.g., a sublingual spray device (FLUMUP SL) vial, pump and actuator.

According to yet another embodiment, the formulation of the invention comprises flumazenil in a concentration of at least 0.2% w/w or flumazenil concentration within the range of 0.2% w/w to 2% w/w, or flumazenil concentration of about 0.4% w/w or flumazenil concentration of about 1.6% w/w.

According to yet another aspect, the invention provides a method of treating excessive sleepiness during waking hours in a subject treated with a sleep drug, the method comprising administering to a subject in need thereof an effective amount of a flumazenil formulation according to the present invention.

It is to be understood that excessive sleepiness during waking hours in a subject treated with a sleep drug includes, but is not limited to, the residual effect of a sleep drug in a subject, the rebound effect of a sleep drug, post awakening drowsiness and the resultant cognitive impairment in a subject treated with a sleep drug, or a treating a disorder selected from: balance impairment induced by a sleep drug and symptoms associated with sleep drug overdose, managing post-sedation drowsiness, for example after general anesthesia in day procedures (like colonoscopy, endoscopy etc.) thereby allowing shorter post-sedation monitoring, the method comprising administering to a subject in need thereof an effective amount of a flumazenil formulation according to the present invention.

The term ‘treating’ as used herein with respect to the treatment of excessive sleepiness during waking hours in a subject treated with a sleep drug using the formulation of the invention, refers to preventing the onset of or ameliorating, the excessive sleepiness during waking hours in a subject treated with a sleep drug by the claimed formulation.

According to one embodiment, the flumazenil formulation of the invention is administered by a route appropriate for self administration, such as: sublingual, subdermal, intranasal, by inhalation and transmucosal, though the aforementioned modes of administration may be used for administration to the patient by others (caretakers). According to yet another embodiment, the flumazenil formulation of the invention is administered repeatedly for as long as treatment is required. In some embodiments, the formulation of the invention is administered several times a day. In other embodiments, the formulation of the invention is administered once a day, or once every other day, or a few times a week, or once a week.

According to yet another embodiment, the flumazenil formulation of the invention is a sublingual flumazenil formulation. According to yet another embodiment, the sublingual flumazenil formulation of the invention is administered to a subject, upon awakening from a sleep drug-induced sleep period.

According to yet another aspect, the invention provides a method for treating alcohol intoxication after alcohol consumption, the method comprising administering to a subject in need thereof an effective amount of a sublingual flumazenil formulation according to the present invention.

According to one embodiment, treating alcohol intoxication includes, but is not limited to, reversing the effects of alcohol intoxication, reducing the effects of alcohol intoxication, alleviating the effects of alcohol intoxication, alcohol withdrawal or improving performance after alcohol consumption.

According to one embodiment, the sublingual flumazenil formulation of the invention is self administered by the subject in need thereof. According to another embodiment, the sublingual flumazenil formulation of the invention is chronically administered, e.g., on a daily basis, e.g., once, twice or thrice daily or more often, as needed. Flumazenil may also be chronically administered on a less frequent basis, e.g., once weekly, twice weekly, once monthly, twice monthly and the like.

Furthermore, it is known that patients suffering from sleep apnea syndrome tend to experience breathing compensation after general anesthesia, including several known cases of post-procedure death from this reason. The compositions of the present invention further may be used for treating/preventing breathing compensation (e.g. hypoventilation) after general anesthesia, particularly in patients with sleep apnea syndrome, e.g. by delivery of flumazenil (e.g., by transdermal, subdermal, sublingual, intranasal or transmucosal routes) with/without automatic monitoring of physiological measures like oxygen saturation or breathing rate.

According to yet another embodiment, the sublingual flumazenil formulation of the invention is administered in a dosing regimen selected from the group consisting of: single administration, repetitive administration and continuous administration.

According to yet another embodiment, the method comprises administering to a subject in need thereof an effective amount of the formulation of the invention, wherein the administering is by any route of delivery suitable for self-administration, thereby treating excessive sleepiness during waking hours in the subject.

According to yet another embodiment, the subject has been diagnosed with a disorder or a disease selected from insomnia and substance addiction, particularly, addiction to benzodiazepines. According to yet another embodiments, the insomnia is selected from the group consisting of sleep onset insomnia; sleep maintenance insomnia; end of sleep insomnia; idiopathic insomnia; hypersomnia; idiopathic hypersomnia; transient insomnia; subacute insomnia; chronic insomnia; Time Zone Change Syndrome, and a combination thereof.

According to yet another embodiment, the sleep drug is selected from the group consisting of a benzodiazepine, a benzodiazepine modulator, a benzodiazepine analog such as a thienodiazepine, a non-benzodiazepine, a 5-HT_{2A} receptor antagonist, a melatonin receptor agonist, an orexin receptor antagonist, a selective serotonin reuptake inhibitor (SSRI), an antihistamine, a herbal product, an immediate release formulation, a controlled release formulation, a sustained release formulation and combinations thereof. In particular embodiments, the sleep drug is a sedative/hypnotic drug. According to yet another embodiment, the sleep drug is a benzodiazepine or a non-benzodiazepine sedative/hypnotic drug.

According to yet another embodiment, the benzodiazepine sleep drug is selected from the group consisting of alprazolam, bromazepam, clonazepam, clotiazepam, cloxazolam, diazepam, estazolam, etizolam, fludiazepam, flunitrazepam, flurazepam, halazepam, haloxazolam, lorazepam, medazepam, midazolam, nimetazepam, nitrazepam, olanzapine, oxazepam, quazepam, temazepam and triazolam. According to yet another embodiment, the non-benzodiazepine sleep drug is selected from the group consisting of adipiplon (NG-2-73), agomelatine, almoxerant (ACT-078573), brotizolam, diphenhydramine, divaplon, doxepin, eplivanserin (SR 46349), doxylamine succinate, eszopiclone, indiplon, ocinaplon, pagoclone, pazinaclone, pruvanserin (EMD 281014), suproclone, suriclone, L-tryptophan, 5-hydroxy-L-tryptophan, melatonin, melatonin receptor agonists, such as VEC-162 and PD-6735, muramyl dipeptide, ramelteon, sleep-promoting substance, uridine, volinanserin (M-100907), zaleplon, zolpidem, APD125, ACP-103, PD 200-390, HY10275, GW649863, and EVT-201. According to yet another embodiment, the thienodiazepine drug is brotizolam.

According to yet another embodiment, the benzodiazepine sleep drug is selected from the group consisting of estazolam, triazolam and temazepam.

According to yet another embodiment, the non-benzodiazepine sleep drug is selected from the group consisting of eszopiclone, zaleplon, indiplon and zolpidem. According to yet another embodiment, the sleep drug is an immediate release formulation, a controlled release formulation or a sustained release formulation.

According to yet another embodiment, the route of administration is sublingual.

According to yet another aspect, the invention provides a method of treating excessive sleepiness during waking hours in a subject, the method comprising administering to a subject in need thereof an effective amount of a flumazenil formulation for providing transdermal delivery.

The excessive sleepiness may be primary (e.g. narcolepsy, idiopathic hypersomnia, etc.) or secondary to other morbidities (e.g. insomnia, sleep apnea syndrome, congestive heart failure, drug overdose) including, improper sleep habits (e.g. lack of sleep).

In particular embodiments, administering for providing transdermal delivery comprises use of a means selected from the group consisting of a patch, an iontophoretic delivery device, a timed-release formulation, ethosomes, liposomes, microneedles and a combination thereof. In particular embodiments, the patch comprises a timed-release formulation. In particular embodiments, the patch comprises at least one of ethosomes, liposomes and microneedles. In particular embodiments, the patch is an iontophoretic patch. In particular embodiments, the patch is a passive patch. In particular embodiments, the patch comprises a mechanical mechanism to initiate the delivery. In particular embodiments, administering for providing subdermal delivery comprises use of a subdermal pump.

In particular embodiments, administering comprises use of a dosage form providing a mode of delivery selected from the group consisting of immediate release, delayed release, pulsatile release, continuous release and repetitive release. In particular embodiments, administering comprises a single dose or multiple doses.

In particular embodiments, administering for providing transmucosal delivery comprises administering to a mucosal surface of the oral cavity or nasal cavity. In particular embodiments, the mucosal surface is selected from the group consisting of the buccal mucosa, the sublingual mucosa, the gingival mucosa, the palatal mucosa, the labial mucosa, the sinusoidal mucosa, the nasal mucosa and a combination thereof.

In particular embodiments, the methods comprise administering flumazenil by a route providing transmucosal delivery. In particular embodiments, the methods comprise self-administering of flumazenil. In particular embodiments, the flumazenil comprises a formulation selected from the group consisting of a mucoadhesive patch, a spray, a liquid drops formulation and a combination thereof.

In particular embodiments, the methods further comprise determining the residual level of sleep drug or metabolite thereof in the subject prior to the administering of the GABA_{A} receptor antagonist thereby optimizing the treatment. In particular embodiments, determining the residual level of sleep drug comprises use of a means selected from the group consisting of: a monitoring device, a kit for measuring drug and/or metabolite levels in a body fluid, a psychomotor test and a combination thereof. In particular embodiments, a patch comprises the monitoring device. In particular embodiments, the monitoring device comprises use of reverse iontophoresis. In particular embodiments, the monitoring device is an electronic monitoring device. In particular embodiments, the body fluid is selected from the group consisting of blood, plasma, serum, saliva, urine, cerebral spinal fluid, semen, tears, mucus and sweat.

In particular embodiments, flumazenil is administered in a dose within the range of about 0.2 to about 10 mg over a 24 hour period (daily). In particular embodiments, flumazenil is administered in an amount of about 4 mg over a 24 hour period. In particular embodiments, flumazenil is administered in an amount in the range of about 0.2 to about 2.0 mg per dose. In particular embodiments, the flumazenil is administered in an amount of about 1.6 mg per dose. In particular embodiments, the flumazenil is administered in an amount of about 0.4 mg per dose. In other particular embodiments, the flumazenil is administered in an amount of about 0.8 mg per dose. In particular embodiments, multiple doses of flumazenil are administered. In particular embodiments, the doses of flumazenil are administered every 30 minutes. In particular embodiments, flumazenil in an amount of 0.4 mg to about 2.0 mg per dose is administered every 30 minutes.

In particular embodiments, the residual effect of a sleep drug comprises one or more of drowsiness, stupor, psychomotor impairment, cognitive impairment, depressed mood, decreased alertness and memory impairment.

In particular embodiments, the methods further comprise administering a wakefulness promoting agent, in combination with flumazenil. In a particular embodiment, the wakefulness promoting agent is selected from the group consisting of modafinil, armodafinil, adrafinil, methylphenidate, venlafaxine, nefazodone, sodium oxybate, phentermine, pemoline, adrenaline, methylxantines, theobromine, caffeine and a combination thereof.

The invention further provides a GABA_{A} receptor antagonist such as flumazenil for use in the treatment of excessive sleepiness due to administration of a sleep drug.

The invention further provides a GABA_{A} receptor antagonist such as flumazenil for use in the treatment of alcohol intoxication after alcohol consumption.

The invention further provides a GABA_{A} receptor antagonist such as flumazenil for use in the treatment of benzodiazepine addiction. The invention further provides a GABA_{A} receptor antagonist such as flumazenil for use in the treatment of excessive sleepiness during waking hours in a subject treated with a sleep drug, by a route of transdermal, transmucosal, sublingual or sublingual delivery.

Other objects, features and advantages of the present invention will become clear from the following description and drawings.

### BRIEF DESCRIPTION OF THE FIGURES

FIG. 1 shows the chemical structure of flumazenil (ethyl 8-fluoro-5,6-dihydro-5-methyl-6-oxo-4H-imidazo[1,5-a](1,4) benzodiazepine-3-carboxylate).

FIG. 2 shows a top-view representation of a transdermal patch according to the invention. The patch has sections A1, A2 and A3 for delivery of drug components A, B and C, respectively.

FIG. 3 shows a vertical cross-section of a transdermal patch according to the invention.

FIG. 4 shows a schematic diagram of the operation of an iontophoretic transdermal delivery device according to the invention.

FIG. 5 is a scheme presenting the preclinical study protocol in rats.

FIG. 6 represents the Mean (±SD) Group Values of Sleeping Time following diazepam induction in group treated with Control Item (Placebo; 3M, 3F), Test Items (F-22; 4M, 4F and F-26; 5M, 5F) and Reference Item (flumazenil USP; 6M, 6F) in male (A) and female (B) rats.

FIGS. 7 and 8 exhibits the Mean (±SD) Group Values of Total distance covered during 15 minutes following diazepam induction in control (3M, 3F), Test Item F-22 (4M, 4F), Test Item F-26 (5M, 5F) and Reference Item flumazenil USP (6M, 6F) in male (FIG. 7) and female (FIG. 8) rats.

FIG. 9 shows the Mean Group Values of Number of Rears during 15 minute measurement following diazepam induction in control (3M, 3F), Test Item F-22 (4M, 4F), Test Item F-26 (5M, 5F) and Reference Item flumazenil USP (6M, 6F) in male (A) and female (B) rats.

FIG. 10 presents the distance of movement at the first (A), second (B) and third time periods (C) and the total distance of movement (D) in the cages, for all animals.

FIG. 11 shows the time spent in the corners of the cages (Area 1; A) or in the central area of the cages (Area 2; B).

FIG. 12 shows changes in ward recall (iWRT score, P=0.005 and 0.002; A), alertness score (P=0.008 and 0.002; B) and DSST score (P=0.01 and 0.008; C) compare to baseline post hypnotic values in a human clinical study (N=20).

FIG. 13 exhibits changes in word recall (iWRT score) compare to baseline post-hypnotic values for the high-dose FLUMUP subpopulation (N=10; P=0.001 and 0.001 accordingly).

FIG. 14 presents the feasibility of passive transdermal delivery of flumazenil (A) and zolpidem (B) from a transdermal 3M paper patch.

FIG. 15 presents the chromatogram of a reverse phase HPLC with a solution of flumazenil.

FIG. 16 presents the correlation between flumazenil concentration and peak area.

FIG. 17 presents gel electrophoresis results for flumazenil at pH 4.5 (A), 5.5 (B) and 7.2 (C).

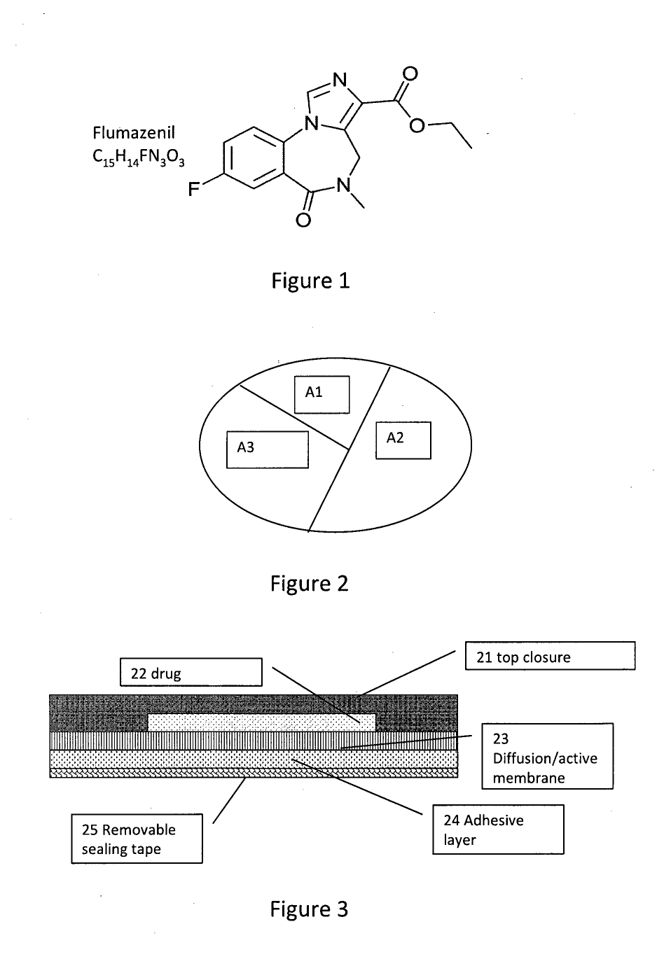

### DETAILED DESCRIPTION OF THE INVENTION

The present invention provides methods of preventing and alleviating excessive and/or residual hypnotic/sleepiness effect in subjects upon wakening from a sleep drug-induced sleep period. The invention can be used for any individual who has been treated with a sleep drug, including those who are diagnosed as insomniac, others who require sleep drugs on a very occasional, infrequent or sporadic basis, and patients undergoing therapeutic or diagnostic medical procedures which requires sedation with sleep/hypnotic drugs. Importantly, the invention addresses the therapeutic goal of achieving a beneficial sleep-waking cycle in insomniac patients by diminishing drowsiness during waking hours when a sedated state is undesirable and enabling normal functioning. Furthermore, the invention addressed the need of reversing drowsiness caused by sleep/hypnotic drugs in patients who have undergone medical procedures requiring general anesthesia and/or sedation with sleep/hypnotic drugs. The invention further provides methods for reversing, reducing or alleviating effects of alcohol intoxication, or improving performance after alcohol consumption and for treating (withdrawal from) benzodiazepine drug addiction. In particular, the methods comprise transmucosal or transdermal administration of an effective amount of a GABA_{A} receptor antagonist, e.g., flumazenil. Currently preferred formulations are sublingual formulations.

### DEFINITIONS

The terms “sleep drug”, “insomnia drug”, “insomnia medication”, “hypnotic drug” and the like are used interchangeably herein in reference to pharmaceutical agents used for inducing and/or maintaining sleep, in particular, prescription sleep drugs that are classified as hypnotics/sedatives. The term “sleep drug”/“hypnotic drug” further encompasses sleep medications given in a Doctor's office or in a hospital setting to induce anesthesia and/or sedation during medical procedures. When taken in excess of the recommended amount, the sleep/hypnotic drugs may cause drug overdose.

The terms “sedation” for medical procedure means any short term sedation, e.g., in operative procedures performed in outpatient clinics or in hospitals.

As used herein, the term “sleep period” refers to a period of time during which sleep continues uninterrupted. The sleep obtained during the sleep period may be perceived by the subject to be restorative sleep or non-restorative sleep.

As used herein, the term “sleep drug-induced sleep period” refers to a sleep period for a discrete period of time induced by administration of a sleep drug. This is in contrast to any residual or side effects (e.g., “hangover effect”, drowsiness) experienced during waking hours following such treatment.

As used herein, the term “discrete period of time” refers to a period of time in which a sleep drug is active, and generally depends upon the half life of the drug.

As used herein, the term “restorative sleep” means sleep which produces a rested state upon waking.

As used herein, the terms “post awakening drowsiness” and “post arousal drowsiness” interchangeably refer to a state characterized by one or more of sleepiness, lethargy, listlessness and a low level of alertness during waking hours.

The terms “wakening” and “awakening” are used herein interchangeably, and encompass the state of being awake and the act of awaking from sleep.

The term “alcohol intoxication” means overdose of alcohol (e.g., ethanol) upon alcohol consumption leading to behavioral impairment. A person is said to suffer from alcohol intoxication when the quantity of alcohol the person consumes exceeds the individual's tolerance for alcohol and produces behavioral, cognitive or physical abnormalities. In other words, the person's mental and physical abilities and performance are impaired.

Alcohol is a generic term for ethanol, which is a particular type of alcohol produced by the fermentation of many foodstuffs—most commonly barley, hops, and grapes. Other types of alcohol commonly available such as methanol (common in glass cleaners), isopropyl alcohol (rubbing alcohol), and ethylene glycol (automobile antifreeze solution) are highly poisonous when swallowed, even in small quantities. Ethanol produces intoxication because of its depressive effects on various areas of the brain causing these impairments in a progressive order as the person gets more and more drunk. Symptoms of alcohol intoxication and/or impaired performance after alcohol consumption include inhibition of normal social functioning (e.g., excessive talking), loss of memory, confusion, disorientation, uncoordinated movement, progressive lethargy, coma, or ultimately death.

As used herein, the term “administering” refers to delivery of a pharmaceutical compound to a subject by any means that does not affect the ability of the compound to perform its intended function. In particular embodiments of the invention, the GABA_{A} receptor antagonist is administered transmucosally, transdermally, by inhalation, sublingually or subdermally.

The term “rebound effect of a sleep drug” as used herein refers to the tendency of the sleep drug, when discontinued, to cause a return of the symptoms being treated by that drug, in a more severe manner than before (i.e. the symptom will be more pronounced after the medication is withdrawn than before it was used).

As used herein, the term “effective amount” refers to an amount of a pharmaceutical compound sufficient to achieve its desired effect.

As used herein, the term “transmucosal” refers to delivery of a pharmaceutical agent to and across a mucosal surface, e.g., sublingually.

As used herein, the term “transdermal” refers to delivery of a pharmaceutical agent through an unbroken skin surface by means of a specific drug delivery system (such as a patch containing a semisolid formulation of the drug) for systemic and/or prolonged drug effect.

As used herein, the term “subdermal” is synonymous with “subcutaneous” and refers to delivery of a pharmaceutical agent by means of a specific drug delivery system that is placed under the skin e.g. an implant, for systemic and/or prolonged drug effect.

As used herein, the term “subject” refers to a mammal, generally a human, to whom a pharmaceutical compound or treatment protocol is administered.

As used herein, the term “iontophoresis” refers to drug delivery across the skin by means of an electric field/current, typically involving two drug-permeated electrodes placed on the skin. Upon application of voltage to the electrodes, the drug migrates through the skin and provides a systemic and/or prolonged effect.

As used herein, the term “treating” encompasses substantially ameliorating, relieving, alleviating and preventing symptoms of a disease, disorder or condition in a subject.

Conditions include side effects caused by administration of one or more pharmaceutical agents, such as for example, a sleep drug, which are other than the desired pharmaceutical effect of the drug. Conditions also include residual effects of a drug/agent that are not longer desired.

As used herein, the term “GABA_{A} receptor antagonist” refers to a compound that binds to but does not activate or fully activate GABA_{A} receptors, thereby inhibiting or blocking the binding and/or action of endogenous γ-aminobutyric acid (GABA) or GABA_{A} receptor agonists.

As used herein, the term “residual effect of a sleep/hypnotic drug” refers to undesired residual effects experienced during waking hours as a result of administration of a sleep/hypnotic drug, including for example, drowsiness, stupor, psychomotor impairment, cognitive impairment, depressed mood, decreased alertness, decreased performance and memory impairment and breathing compensation. Residual effect of a sleep/hypnotic drug in the context of the present invention also includes the rebound effect of the sleep drug.

As used herein, the term “insomnia” refers to a sleep disorder characterized by the subjective perception of dissatisfaction with the amount and/or quality of sleep. Forms of insomnia include but are not limited to: sleep onset insomnia, also termed “initial insomnia” (difficulty in falling asleep); sleep maintenance insomnia, also termed “middle insomnia” (difficulty in remaining asleep); end of sleep insomnia, also termed “terminal insomnia” (early awakening, typically coupled with the inability to fall asleep again); idiopathic insomnia (a chronic inability to obtain adequate sleep, manifest for example as initial insomnia, middle insomnia, or both); transient insomnia, also termed “adjustment sleep disorder” (sleep disturbance temporally related to stress, conflict, or environmental change causing emotional agitation); Time Zone Change Syndrome also termed “jet lag” (varying degrees of insomnia, generally accompanied by difficulty in waking up, excessive sleepiness, decrements in subjective daytime alertness and performance, due to rapid travel across multiple time zones).

Insomnia can be classified based on different concepts, including duration, severity and form of presentation. Transient or acute insomnia persists for less than 4 weeks; short-term or subacute insomnia persists for longer than 4 weeks but less than 3 to 6 months, and long-term or chronic insomnia persists for longer than 3 to 6 months. In terms of severity, insomnia may be mild, moderate or severe. While each of these forms of insomnia may occur almost every night, mild insomnia is associated with a minimum impairment of quality of life, while moderate and severe insomnia are associated with increasing degrees of impairment of quality of life, due to associated symptoms e.g. irritability, anxiety, fatigue. Forms of presentation of insomnia include sleep onset insomnia i.e. difficulty in initiating sleep; sleep maintenance insomnia i.e. difficulty in maintaining sleep, and end of sleep insomnia i.e. early awakening coupled with the inability to fall asleep again. Each of the aforementioned conditions requires a different therapeutic goal, and the duration of the effect of any particular sleep drug/agent is an important parameter to be considered upon choosing a sleep drug for therapy. For example, for treatment of sleep onset insomnia, a drug with a short half life is suitable, while for treatment of sleep maintenance insomnia and end of sleep insomnia, a drug with a longer half life is more appropriate for providing a longer lasting sedative effect.

As used herein, the term “hypersomnia” refers to chronic or recurrent bouts of excessive sleepiness, characterized by one or more of near-daily diurnal sleep episodes, excessive naps, abnormally prolonged sleep intervals, a perception of non-restorative sleep, and difficulty in making the transition from sleep to wakefulness. Hypersomnia may be one or more of: shift work sleep disorder; narcolepsy; obstructive sleep apnea/hypopnea syndrome; REM behavior disorder; frontal nocturnal dystonia; restless legs syndrome; nocturnal movement disorder; Kleine-Levin syndrome; Parkinson's disease; excessive sleepiness; hypersomnia; idiopathic hypersomnia; recurrent hypersomnia; endozepine related recurrent stupor; and amphetamine resistant hypersomnia.

The terms “composition,” “formulation” and “dosage form” are used herein interchangeably to encompass formulated preparations comprising one or more pharmacologically active drugs, and one or more pharmaceutically acceptable excipients, diluents or carriers. Compositions, formulations and dosage forms can be designed for administration by all possible administration routes to achieve the desired therapeutic response. The terms used may refer to the physical format of the product which is dispensed and administered to the patient, for example, a capsule or a patch. Alternately or in addition, the terms used may refer to any of: the mode of administration, the mode of delivery or the mode of release of the drug, for example a transdermal delayed release formulation. By “pharmaceutical” or “pharmaceutically acceptable” it is meant that any excipient, diluent or carrier in the composition, formulation, or dosage form is compatible with the active ingredient and not deleterious to the recipient thereof.

The terms “timed-release” and “delayed release” are used herein interchangeably to refer to a pharmaceutical dosage form in which release of the active ingredient is other than promptly after administration of the dosage form, but rather is withheld or delayed following administration. The terms “lag time” and “delay” refer to the time span from the point of administration of the dosage form to the point at which the active ingredient becomes bioavailable and/or exerts a pharmacological effect.

As used herein the terms “sleep drugs” or “hypnotic drugs” or “sleep/hypnotic drugs” are interchangeably used herein to refer primarily to benzodiazepines which constitute a well-known class of therapeutics displaying hypnotic, anxiolytic and anticonvulsant effects. This class includes the sleep inducing hypnotics, brotizolam (Bondormin®), a benzodiazepine, and zolpidem (Stilnox®), a non benzodiazepine.

Sleep/hypnotic drugs are commonly used to reduce tension and anxiety and induce calm (sedative effect) or to induce sleep (hypnotic effect). Most such drugs exert a quieting or calming effect at low doses and a sleep-inducing effect in larger doses. Sedative-hypnotic drugs tend to depress the central nervous system (CNS). Since these actions can be obtained with other drugs, such as opiates, the distinctive characteristic of sedative-hypnotics is their selective ability to achieve their effects without affecting mood or reducing sensitivity to pain. Gamma-Aminobutyric acid (GABA) is the major inhibitory neurotransmitter in the mammalian CNS. GABA participates in the regulation of neuronal excitability through interaction with specific the GABAA receptors. The binding of GABA to these postsynaptic receptors, results in an opening of a chloride channel integrated in the receptor which allows the entry of chloride and consequently leads to hyperpolarization of the recipient cell. The action of GABA is allosterically modulated by a wide variety of chemical entities which interact with distinct binding sites at the GABAA receptor complex. One of the most thoroughly investigated modulatory site is the benzodiazepine binding site.

The terms “controlled release” and “sustained release” are used herein interchangeably to refer to a pharmaceutical dosage form in which release of the active ingredient is at a rate sufficient to maintain the desired therapeutic level over an extended period of time.

As used herein, the term “buccal tablets” refers to tablets, typically small, flat and soft tablets, which are designed to be placed in the side of the cheek (i.e. buccal cavity) to be directly absorbed through the buccal mucosa for a systemic effect.

As used herein, the term “oral films” refers to films administered on the gyngiva or tounge or buccal.

As used herein, the term “sublingual tablets” refers to tablets, typically small, flat and soft tablets, which are designed to be placed under the tongue to be directly absorbed through the sublingual mucosa for a systemic effect.

As used herein, the term “sublingual spray” refers to a formulation for delivery to the sublingual mucosa in the form of a spray for a systemic effect, typically provided in spray actuators, designed to access the mucosal surfaces under the tongue or the lips.

As used herein, the term “half life” in reference to a drug refers to the time required to eliminate, decompose or metabolize 50% of the initial amount of drug. Thus the higher the half-life, the longer the drug is present in the body.

The singular forms “a”, “and”, and “the” include plural referents unless the context clearly dictates otherwise. Thus, for example, reference to “a sleep drug” includes a plurality of such drugs, and so on.

The terms “chronic usage”, “continuous usage”, “chronic administration” or “continuous administration” are interchangeably used herein to describe a repetitive use of a pharmaceutical formulation, specifically, the flumazenil formulation of the invention, namely, repetitive dosages a few times a day (e.g. every 30 minutes, every hour), or once a day, or several times a week, or once a week, and the like, for as long as treatment is required.

### Compositions of the Invention

In some aspects, the present invention provides a liquid formulation for sublingual administration, the formulation comprising flumazenil as an active ingredient, a solubilizing agent selected from an alcohol, a glycol and a combination thereof, a cyclodextrin, a buffering agent, a penetration enhancer and optionally a preservative.

The present invention provides a pharmaceutical composition comprising a GABA_{A} receptor antagonist. A GABA_{A} receptor antagonist is a negative allosteric modulator. The GABA_{A} receptor antagonist may be selected from the group consisting of: flumazenil; clarithromycin; picrotoxin; bicuculline; cicutoxin and oenanthotoxin. Preferably, the GABA_{A} receptor antagonist is flumazenil.

In particular embodiments, the GABA_{A} receptor antagonist is administered at least 2 hours after the administration of a sleep drug. In particular embodiments, the GABA_{A} receptor antagonist is administered about 4 to 8 hours after the administration of the sleep drug. In particular embodiments, the GABA_{A} receptor antagonist is administered about 4 hours after the administration of a sleep drug. In particular embodiments, the GABA_{A} receptor antagonist is administered about 6 hours after the administration of the sleep drug. In particular embodiments, the GABA_{A} receptor antagonist is administered at any time that is desired by the user after the administration of the sleep drug.

In particular embodiments, the GABA_{A} receptor antagonist is administered together with the sleep drug, wherein the GABA_{A} receptor antagonist is administered as a timed-release formulation. In particular embodiments, the GABA_{A} receptor antagonist and the sleep drug are provided together in a single formulation, wherein the formulation provides the sleep drug for immediate release and wherein the formulation provides the GABA_{A} receptor antagonist for timed-release.

In particular embodiments, the GABA_{A} receptor antagonist is administered immediately upon awakening from a sleep drug-induced sleep period. In particular embodiments, the GABA_{A} receptor antagonist is administered within up to about four hours from awakening from a sleep drug-induced sleep period. In particular embodiments, the GABA_{A} receptor antagonist is administered within about two hours from awakening. In particular embodiments, the GABA_{A} receptor antagonist is administered within about one hour from awakening. In particular embodiments, the GABA_{A} receptor antagonist is administered within about 30 minutes from awakening. In particular embodiments, the GABA_{A} receptor antagonist is administered within about 10 minutes from awakening. In particular embodiments, the GABA_{A} receptor antagonist is administered within about 5 minutes from awakening.

In particular embodiments, the method comprises self-administering of the GABA_{A} receptor antagonist.

Flumazenil

Flumazenil (ethyl 8-fluoro-5,6-dihydro-5-methyl-6-oxo-4H-imidazo[1,5-a] [1,4]benzodiazepine-3-carboxylate), an imidazobenzodiazepine having high affinity for the GABA_{A}/benzodiazepine-receptor complex, that is the specific binding site of benzodiazepines, is a competitive inhibitor of benzodiazepines. Thus, flumazenil is used to reverse benzodiazepine-induced sedation and anesthesia following therapeutic or diagnostic procedures (see for example, Goldfrank, L. R. (2002) Goldfrank's toxicologic emergencies. New York: McGraw-Hill). In addition, flumazenil reverses the effect of non-benzodiazepine drugs, such as the imidazopyridine hypnotic zolpidem (see for example Patat et al., Clin Pharmacol Ther. 1994 October; 56(4):430-6). Flumazenil (Romazicon®) was approved to date for the complete or partial reversal of the sedative effects of benzodiazepines in cases where general anesthesia has been induced and/or maintained with benzodiazepines, where sedation has been produced with benzodiazepines for diagnostic and therapeutic procedures, and for the management of benzodiazepine overdose. The current usage of flumazenil known in art is sporadic, i.e. on specific occasions as needed to treat medical conditions and not on a daily basis. Since the commercial form of flumazenil is a liquid dosage form for injection, the use of flumazenil as known in art requires a physician/health provider, rather than a self-administration, as described in herein.

The onset of flumazenil action is rapid and usually takes effect within few seconds to two minutes, with a peak effect occurring six to ten minutes post administration. The recommended intravenous dose for adults is 200 μg every 1 to 2 minutes until the benzodiazepine-reversal effect is seen to a maximum of 3 mg per hour. Currently approved formulations of flumazenil are solutions intended for intravenous administration, such as Romazicon® (NDA 20-073/S-016) which contains in each mL: 0.1 mg flumazenil, 1.8 mg methylparaben, 0.2 mg propylparaben, 0.9% sodium chloride, 0.01% edentate disodium and 0.01% acetic acid where pH is adjusted to approximately 4 with hydrochloric acid and/or, if necessary, sodium hydroxide. The recommended doses and titration rates for Romazicon® are 0.2 mg to 1 mg given at 0.2 mg/min and for repeat treatments no more than 3 mg should be given in any one hour.

Flumazenil administered by i.v. infusion has been disclosed to be useful in treatment of overdose and toxification with the sleep drug zolpidem (see for example, Lheureux et al., Hum Exp Toxicol. 1990 March; 9(2):105-7; Quaglio et al., Int Clin Psychopharmacol. 2005 September; 20(5):285-7).

Flumazenil has been disclosed to be effective for reversing triazolam and zolpidem-induced memory impairment (Wesensten et al., Psychopharmacology (Berl). 1995 September; 121 (2):242-9).

Flumazenil administered intranasally in combination with naloxone has been disclosed to be effective for treating a pediatric patient over-sedated with midazolam and sufentanil during dental treatment (Heard et al., Paediatr Anaesth. 2009 August; 19(8):795-7).

Flumazenil has been disclosed to be effective for reversing the central nervous system depressant effects of midazolam in children undergoing conscious sedation (Shannon et al., J. Pediatr. 1997 October; 131(4):582-6).

Radiolabeled forms of flumazenil, such as ¹⁸F-flumazenil have been disclosed to be useful as tracers in positron emission tomography (PET) for investigating neurological and psychiatric disorders, including ischemic cerebral artery stroke (see for example, Massaweh et al., Nucl Med Biol. 2009 October; 36(7):721-7).

Oral delivery has shown low bioavailability of 16% due to high hepatic elimination (Als-Nielsen et al., Cochrane Database Syst Rev. 2004:2:CD002798). The effective plasma level is 5-20 ng/ml (for IV 0.2 mg) while oral dose of 200-600 mg produced plasma level of 143-439 ng/ml level with a peak plasma level within 20-90 minutes from the oral administration (e.g. Barbaro et al., Hepatology, 1998, 28:374-378). The protein binding of the drug is 40%, its volume distribution is 0.63-1.6 L/kg. The plasma half life time is 7-15 minutes, but its brain effect or biological half life is 35.3±13.8 minutes.

Without wishing to be bound by any theory or mechanism, it is understood that flumazenil selectively antagonizes or attenuates the effects of benzodiazepines in the CNS by competitively inhibiting their actions at the benzodiazepine binding site of the gamma aminobutyric acid (GABA)-benzodiazepine receptor complex. Flumazenil does not antagonize the effects of CNS-active substances that act via other receptors. Also, flumazenil does not alter the pharmacokinetics of benzodiazepines. The extent to which Flumazenil reverses the effects of a benzodiazepine depends on the dose and plasma concentration of both medications and on the effect being assessed. Flumazenil reverses some components of benzodiazepine-induced hypoventilation, leading to at least partial improvement in respiratory function. Also, amnesia is antagonized less consistently and less completely than psychomotor deficits, which may be reversed less completely than sedation.

Cyclodextrins:

Cyclodextrins are relatively large molecules (molecular weight ranging from almost 1000 to over 1500), with a hydrated outer surface, and under normal conditions, cyclodextrin molecules will only permeate biological membranes with considerable difficulty. It is generally recognized that cyclodextrins act as true carriers by keeping the hydrophobic drug molecules in solution and delivering them to the surface of the biological membrane, e.g. skin, mucosa or the eye cornea, where they partition into the membrane. The relatively lipophilic membrane has low affinity for the hydrophilic cyclodextrin molecules and therefore they remain in the aqueous membrane exterior, e.g. the aqueous vehicle system, salvia or the tear fluid. Conventional penetration enhancers, such as alcohols and fatty acids, disrupt the lipid layers of the biological barrier. Cyclodextrins, on the other hand, act as penetration enhancers by increasing drug availability at the surface of the biological barrier.

The expression “cyclodextrin” as used herein means α-, β- or γ-cyclodextrin or a derivative thereof. Suitable cyclodextrin derivatives for use in the formulations of the present invention include, but are not limited to the cyclodextrin listed above, e.g. hydroxypropyl derivatives of α-, β- and γ-cyclodextrin, sulfoalkylether cyclodextrins such as sulfobutylether β-cyclodextrin, alkylated cyclodextrins such as the randomly methylated β-cyclodextrin, and various branched cyclodextrins such as glucosyl- and maltosyl β-cyclodextrin (e.g. T. Loftsson and M. E. Brewster, “Cyclodextrins as pharmaceutical excipients”, Pharm. Technol. Eur., 9(5), 26-34 (1997); the contents of which are incorporated by reference in their entirety). Other cyclodextrins are described in US patent publication US 2004/0186075, the contents of which are incorporated by reference in their entirety.

Addition of organic solvents, such as ethanol, to the aqueous complexation media can result in enhanced complexation efficiency. Furthermore, addition of certain low molecular weight acids, such as acetic, citric, malic, or tartaric acid, to aqueous complexation media can further enhance cyclodextrin solubilization of basic drugs.

In one currently preferred embodiment, the cyclodextrin is hydroxypropyl β-cyclodextrin (HPCD). The cyclodextrin is preferably formulated in a buffer having a pH from about 3 to about 6. In one particular embodiment, the cyclodextrin (e.g., HPCD) is formulated in a citric acid buffer having a pH of about 4.

The cyclodextrin component of the formulations of the present invention can be present in an amount from about 10% to about 95% w/w, for example from about 30% to about 80%, from about 30% to about 75%, or preferably about 60% based on the formulations of the invention.

Solubilizing Agents:

The solublizing agent is preferably a polar solvent such as mono- or poly-alcohols of linear or branched configuration (e.g., C1 to C8 alcohols). Non-limiting examples include methanol, ethanol, propanol, iso-propanol, n-butanol, sec-butanol, isobutanol, t-butanol, n-pentanol, 2-pentanol, 3-pentanol, neopentanol, 1-hexanol, 2-hexanol, 3-hexanol, 1-heptanol, 2-heptanol, 1-octanol, as well as any geometrical isomers, enantiomers and diastereomers of any of the foregoing. Other suitable polar solvents include glycols such as ethylene glycol, propylene glycol and their polymers having a molecular weight between 400 and 1000. Each possibility represents a separate embodiment of the present invention. In one particular embodiment, the solubilizing agent is a combination of ethanol and propylene glycol.

The solubilizing component of the formulations of the present invention can be present in a total amount from about 10% to about 95% w/w, for example from about 30% to about 80%, from about 30% to about 75%, or preferably about 30% or 40% based on the formulations of the invention.

Penetration Enhancers:

The formulations of the present invention further comprise penetration enhancers, preferably skin/mucosal penetration enhancers such as menthol. It has been surprisingly found that the inclusion of penetration enhancers, e.g., menthol, significantly improve the performance of the formulations in the utilities described herein, as opposed to conventional formulations which lack such excipient.

Other penetration enhancers that may be used in the formulations of the present invention include, but are not limited to anionic surfactants (e.g. sodium lauryl sulphate, sodium dodecyl sulphate), cationic surfactants (e.g. palmitoyl DL camitine chloride, cetylpyridinium chloride), nonionic surfactants (e.g. polysorbate 80, polyoxyethylene 9-lauryl ether, glyceryl monolaurate, polyoxyalkylenes, polyoxyethylene 20 cetyl ether), lipids (e.g. oleic acid), bile salts (e.g. sodium glycocholate, sodium taurocholate), chitosan or a chitosan derivative, linalool, carvacrol, thymol, citral or t-anethole, and related compounds.

Preservatives:

The formulations of the invention optionally further comprise at least one preservative. Any suitable preservative may be present in the formulation in the present invention, in particular a preservative that prevents microbial spoilage of the liquid solution. The preservative may be any pharmaceutically acceptable preservative, for example methyl 4-hydroxybenzoate (methyl paraben), ethyl 4-hydroxybenzoate (ethyl paraben), propyl 4-hydroxybenzoate (propylparaben), benzyl alcohol, sorbic acid, sodium benzoate, benzoic acid, and any combination thereof. The parabens are preferably used in combination.

Currently preferred preservatives are selected from benzyl alcohol, propylparaben, methylparaben and combinations thereof. In one embodiment, the preservative is benzyl alcohol. In another embodiment, the preservative is a combination of propylparaben and methylparaben. Each possibility represents a separate embodiment of the present invention.

The preservative may be present in an amount ranging from 0.1% to 30% w/w, for example from about 1% to about 15%, for example about 1%, about 2%, about 5% or about 10% based on the weight of the formulation.

Additional Excipients:

The formulation of the invention optionally further comprises a flavoring agent in an amount between 0.05 and 10 percent by weight of the total composition. In one embodiment, the flavoring agent is present in an amount between 0.1 and 2.5 percent by weight of the total composition. The flavoring agent is preferably selected from the group consisting of synthetic or natural oil of peppermint, oil of spearmint, citrus oil, fruit flavors, sweeteners (sugars, aspartame, saccharin, Estevia, etc.), and mixtures thereof. Menthol can also act as a flavoring agent.

In one embodiment, the formulation is provided in the form of a sublingual spray, e.g., a sublingual spray device (FLUMUP SL) vial, pump and actuator.

### The Compositions and Methods of the Invention

The present invention provides a unique pharmaceutical formulation which was shown to be effective in countering the sedative effects induced by sleep/hypnotic drugs, when administered in a sublingual route of administration. The formulation of the invention is different by many aspects from known flumazenil formulations. First, the formulation of the invention comprises flumazenil in a concentration that is exceptionally high. The concentration of flumazenil according to the principles of the present invention is at least 0.2%, within the range of 0.2-2% and in particular embodiments it is 0.4% w/w or 1.6% w/w, while the concentration of Flumzenil in the commercially available formulation of Romazicon® is only 0.01% w/w. Nevertheless, the formulation of the invention was shown to be safe. In addition, the formulation of the invention was shown to be more effective in countering excessive sleepiness induced by sleep drugs than the commercial formulation of flumazenil (which is intended for i.v. administration), when both formulations are administered sublingually (see, for example, Example 17 herein below and Eddy et al., ibid). The formulation of the invention was also shown to be advantageous over a non-formulated flumazenil USP solution which merely includes a solubilizing agent (Tween 80) and saline. This advantage may be attributed to the special combination of excipients of the claimed formulation. Specifically, the formulation of the invention comprises a unique combination of excipients, including, a solubilizing agent selected from an alcohol, a glycol and a combination thereof, a buffering agent, and at least one agent or a plurality of agents, selected from the group consisting of: a penetration enhancer, a surfactant, cyclodextrin, a solubilizing agent and a preservative.

Unexpectedly, the strong countering effect exhibited by the formulation of the invention is maintained for at least 60 minutes after administration in humans. In fact, the anti-sedating effect of the formulation of the invention is enhanced and is more effective an hour after administration as compared with 20 minutes after administration.

A desired secondary effect of the formulation of the invention is that it is effective in reducing balance impairment accompanied with hypnotics and therefore it is also beneficial in decreasing falls and subsequent bone fractures in elderly using sleeping/hypnotic drugs, a known cause of significant morbidity and mortality in this age group

For carrying out the methods of the invention, the GABA_{A} receptor antagonist to be administered may be selected from flumazenil; clarithromycin; picrotoxin; bicuculline; cicutoxin; and oenanthotoxin. In a currently preferred embodiment, the GABA_{A} receptor antagonist is flumazenil.

The methods of the invention are used in conjunction with insomnia treatment modalities, and serve to eliminate or diminish residual soporific effects associated with administration of sleep drugs. The invention is effective for counteracting excessive sleepiness induced by a wide variety of sleep drugs. Such sleep drugs include benzodiazepine and non-benzodiazepine drugs which are classified as hypnotics/sedatives, as well as other prescription and non-prescription sleep drugs, including those classified as 5-HT_{2A} receptor antagonists, melatonin receptor agonists, orexin receptor antagonists, selective serotonin reuptake inhibitors (SSRIs), and other sleep inducing agents such as antihistamines, melatonin and certain herbal products. It is to be specifically understood that a particular sleep drug may be classified under more than one of the aforementioned categories.

Benzodiazepine drugs include those classified as 1,4-benzodiazepines, 1,5-benzodiazepines, 2,3-benzodiazepines, triazolobenzodiazepines, imidazobenzodiazepines, oxazolobenzodiazepines, thienodiazepines, pyridodiazepines, pyrazolodiazepines, pyrrolodiazepines, and benzodiazepine prodrugs. Benzodiazepine drugs include, without limitation, alprazolam, bromazepam, clonazepam, clotiazepam, cloxazolam, diazepam, estazolam, etizolam, fludiazepam, flunitrazepam, flurazepam, halazepam, haloxazolam, lorazepam, medazepam, midazolam, nimetazepam, nitrazepam, olanzapine, oxazepam, quazepam, temazepam and triazolam. In a particular embodiment, the benzodiazepine sleep drug may be selected from estazolam, triazolam and temazepam.

In addition, benzodiazepine analogs such as thienodiazepines (e.g. brotizolam), may be used in the context of the present invention.

Non-benzodiazepine drugs include, without limitation, adipiplon (NG-2-73), agomelatine, brotizolam, divaplon, eszopiclone, indiplon, ocinaplon, pagoclone, pazinaclone, suproclone, suriclone, ramelteon, zaleplon, zolpidem (Intermezzo®), PD 200-390, and EVT-201. In a particular embodiment, the non-benzodiazepine sleep drug may be selected from eszopiclone, zaleplon and zolpidem.

5-HT_{2A} receptor antagonists include, without limitation, doxepin, eplivanserin (SR 46349), pruvanserin (EMD 281014), HY10275, APD125, ACP-103 and volinanserin (M-100907). Melatonin receptor agonists include, without limitation, VEC-162 and PD-6735. Non-benzodiazepine sleep drugs also include selective serotonin reuptake inhibitors (SSRI). Orexin receptor antagonists include for example, almoxerant (ACT-078573) and GW649863.

Antihistamines may be used for inducing sleep, and include, without limitation, diphenhydramine, doxylamine succinate, loratadine, desloratadine, meclizine, fexofenadine, pheniramine, cetirizine, promethazine, chlorpheniramine and levocetirizine.

Other agents used for inducing sleep include, without limitation, L-tryptophan, 5-hydroxy-L-tryptophan, melatonin, muramyl dipeptide, sleep-promoting substance (see for example, Inoue et al Proc Acad Natl Sci USA October 1984, 81:6240-6244) and uridine.

The sleep drug may comprise an herbal product, for example, valerian, linden, kava, chamomile, catnip, passionflower, or a combination thereof.

In a particular embodiment, the sleep drug is an immediate release formulation, a controlled release formulation or a sustained release formulation.

Administration of the GABA_{A} receptor antagonist preferably comprises self administration in accordance with a physician's instructions and under appropriate supervision, using a formulation that provides transmucosal, transdermal or sublingual delivery.

In the methods of the invention, the GABA_{A} receptor antagonist may be administered before, and/or upon awakening from a sleep drug-induced sleep period, either immediately upon awakening, or during later periods of activity when the patient is experiencing drowsiness. For example, the GABA_{A} receptor antagonist may be administered within about four hours, or within about two hours, or within about one hour, or within about 30 minutes, or within about 10 minutes, or within about 5 minutes from awakening from a sleep drug-induced sleep period.

In addition, the formulation used for administration may provide immediate or delayed release of the GABA_{A} receptor antagonist, and the release mode may be any of pulsatile, continuous or repetitive. Single or multiple doses may be provided.

The GABA_{A} receptor antagonist may be administered about 4 to 8 hours after the administration of the sleep drug, such as about 6 hours after the administration of the sleep drug. In alternate embodiments, the GABAA receptor antagonist may be administered at the same time as the sleep drug, with the condition that the GABA_{A} receptor antagonist is administered as a timed-release formulation. This means that the GABA_{A} receptor antagonist will exert its effect only after a suitable period of time has elapsed after a sleep drug administration e.g. 6 hours, thus enabling the patient to experience an adequate sleep period induced by the sleep drug. For this embodiment, the GABA_{A} receptor antagonist and the sleep drug may be provided in separate dosage forms, or together in a single dosage form which incorporates the different types of formulations. In both cases however, the sleep drug and the GABA_{A} receptor antagonist should be formulated so that their peak periods of efficacy do not coincide. For example, the sleep drug is formulated for immediate or controlled release, while the GABA_{A} receptor antagonist is formulated for delayed-release. For example, an oral dosage form providing immediate release of a sleep drug may be administered in the evening before bedtime, and at the same time a transdermal patch containing the GABA_{A} receptor antagonist as a delayed release formulation may be applied to the skin e.g. upper thigh. Alternately, a single transdermal patch containing the sleep drug as an immediate release formulation or as a controlled release formulation, and the GABA_{A} receptor antagonist as a delayed-release formulation may be used. Particular embodiments of such transdermal patches are disclosed herein in Example 9.

The invention further provides a patch for transdermal delivery of flumazenil, wherein the patch provides transdermal delivery of the flumazenil with a lag time of 5 to 7 hours from the time of application. In particular embodiments, the patch comprises a first compartment comprising a sleep drug and a second distinct compartment comprising the flumazenil, wherein the patch provides transdermal delivery of the sleep drug with a lag time of 1 to 2 hours from the time of application and further provides controlled release of the sleep drug.

The methods of the invention are directed to reducing the residual effects of sleep drugs following administration of such drugs to diagnosed insomniac patients, and to other individuals who require sleep drugs on an occasional ad hoc or infrequent basis to overcome transient difficulties in falling asleep, maintaining sleep, or achieving restorative sleep. In diagnosed insomniac patients, the type of insomnia may be any of the various forms of that sleep disorder, including for example, sleep onset insomnia; sleep maintenance insomnia; end of sleep insomnia; idiopathic insomnia; transient insomnia; subacute insomnia; chronic insomnia; Time Zone Change Syndrome (“jet lag”), and a combination thereof.

The invention further provides use of a GABA_{A} receptor antagonist such as flumazenil for manufacturing a medicament for treating excessive sleepiness due to administration of a sleep drug, wherein the medicament is formulated for transmucosal, transdermal or sublingual delivery of the GABA_{A} receptor antagonist.

Further provided is use of a GABA_{A} receptor antagonist such as flumazenil for the manufacture of a medicament for preventing or alleviating post awakening drowsiness due to administration of a sleep drug, wherein the medicament is formulated for transmucosal, transdermal or sublingual delivery of the GABA_{A} receptor antagonist.

Further provided is use of a GABA_{A} receptor antagonist such as flumazenil for the manufacture of a medicament for reversing the residual effect of a sleep drug, wherein the medicament is formulated for transmucosal, transdermal or sublingual delivery of the GABA_{A} receptor antagonist.

The methods disclosed herein are effective for counteracting the residual effects of a sleep drug experienced during waking hours, including for example, any of drowsiness, stupor, psychomotor impairment, cognitive impairment, depressed mood, decreased alertness, decreased performance and memory impairment, subjective and/or objective.

Also, the methods disclosed herein are effective for preventing or alleviating post awakening drowsiness in a subject treated with a sleep/hypnotic/sedative drug, such as during diagnostic or therapeutic procedures. As demonstrated herein, the formulations of the present invention, which are formulated for sublingual administration, are unexpectedly superior to known flumazenil formulations given intravenously for alleviating drowsiness caused by sleep/hypnotic drugs, or for managing sleep drug overdose.

The efficacy of the methods described herein for counteracting such effects may be assessed, for example by direct observation of behavioral and physiological properties, by checking walking and/or standing stability, by self-reporting, and/or by various well-known electrophysiological methods and performance skill methods. Such methods include, for example, examining electroencephalograph (EEG) activity amplitude and frequency patterns, examining electromyogram activity, and examining the amount of time during a measurement time period, in which a mammal is awake or exhibits a behavioral or physiological property characteristic of wakefulness.

Objective and subjective tests for wakefulness, alertness and performance include, for example, the Epworth Sleepiness Scale; the Stanford Sleepiness Scale; the Pittsburgh Sleep Quality Index; an Activity-Rest and Symptom Diary; Actigraphy; Psychomotor Vigilance Task; Polysomnography; Functional Magnetic Resonance Imaging; Profile of Mood States; Functional Outcomes of Sleep Questionnaire; Medical Outcomes Study Short-Form 36; Cambridge Neurophysical Test Automated Battery (CANTAB, including e.g., physcomotor speed, attention, working memory, and executive function); and PAB Battery.

Additional methods used to monitor or assess alertness/drowsiness levels in a subject prior to and following use of the methods disclosed herein may employ various devices for measurement of eye position or closure, assumed to correlate with alertness/drowsiness, as disclosed for example in U.S. Pat. Nos. 5,689,241; 5,682,144 and 5,570,698.

The methods of the invention may further comprise a step of determining the residual level of a sleep drug or a metabolite thereof in the subject prior to the step of administering the GABA_{A} receptor antagonist. Such determinations may be carried out using a monitoring device, a kit for measuring drug and/or metabolite levels in a body fluid, a psychomotor test or a combination thereof. Commercial available kits for detecting the presence of benzodiazepine metabolites include the QuickScreen™ One-Step Rapid Benzodiazepine Test, marketed by Craig™ Medical Distribution Inc.

Typically, such kit technology incorporates a chromatographic absorbent device in which the drug or drug metabolites in the body fluid sample compete with a benzodiazepine or derivative immobilized on a porous membrane for limited antibody sites. In the assay, the sample of body fluid (usually urine) mixes with labeled antibody-dye conjugate and migrates through the test device. When the benzodiazepine level in the sample is below the detection cutoff sensitivity of the test e.g. 200 ng/ml, unbound antibody-dye conjugate binds to immobilized antigen conjugate, producing a signal of a particular color e.g. red or pink, that indicates a negative result. Conversely, when the benzodiazepine level is above the detection limit threshold, antibody-dye conjugate binds to the free drug, forming an antigen-antibody-dye complex, and preventing the development of the color signal. Such kits usually further include a non-specific sandwich dye conjugate reaction for quality control to demonstrate antibody recognition, verifying that the reagents are chemically active.

In particular embodiments, the body fluid used for determining the residual level of a sleep drug is selected from blood, plasma, serum, saliva, urine, cerebral spinal fluid, semen, tears, mucus or sweat.

In other embodiments, determining the residual level of a sleep drug is carried out using a monitoring device, such as a patch (e.g. by reverse iontophoresis). Patches for monitoring drug levels in blood are disclosed for example in U.S. Pat. Nos. 5,817,012; 5,291,887 and 5,113,860. In other embodiments, the monitoring device may be an electronic monitoring device

Alternately or in addition, the determination of the residual level of the sleep drug or a metabolite thereof may be carried out using qualitative assessments, for example, a psychomotor test, as described above.

### Sublingual, Transmucosal and Transdermal Administration and Formulations

It has further been discovered that the sublingual formulations of the present invention containing GABA_{A} receptor antagonist, especially flumazenil, are useful in reversing, reducing or alleviating effects of alcohol intoxication (i.e., a drunken state), or improving performance after alcohol consumption. Preferably, the formulation is self-administered by the subject in need thereof. It is known that ethanol acts in the central nervous system by binding to the GABA_{A} receptor, increasing the effects of the inhibitory neurotransmitter GABA (i.e., it is a positive allosteric modulator). Flumazenil, being a GABA_{A} receptor antagonist, alleviates the effects caused by alcohol (ethanol) intoxication and improves performance after alcohol consumption.

In one embodiment the present invention provides a method to alleviate excessive sleepiness and drowsiness during waking hours when a sedated state is undesirable and is moreover counterproductive to a therapeutic goal including the goal of achieving beneficial sleep-waking cycles, in insomniac patients, using flumazenil which is administered via a sublingual spray, one of the most convenient routes of administration for self use.

Transmucosal administration refers to delivery of a pharmaceutical agent to and across the mucous membranes, in particular of the oral cavity (i.e., oral mucosa) and of the nasal cavity. Examples of suitable sites of administration within the oral mucosa include the mucous membranes of the floor of the mouth (sublingual mucosa), the upper part of the throat below the soft palate and above the larynx (pharyngeal mucosa), the cheeks (buccal mucosa), the gums (gingival mucosa), the roof of the mouth (palatal mucosa), the lining of the lips (labial mucosa) and combinations thereof.

Examples of suitable sites of transmucosal administration within the nasal cavity include the nasal mucosa and the sinusoidal mucosa.

Transmucosal administration allows for rapid absorption and ready bioavailability of a drug, and avoids the first pass effect of hepatic metabolism. Further, it is pain-free and ideally suited for self-administration.

Transmucosal delivery formulations include those in the form of a spray, a tablet, an oral film, a lozenge, a film, a powder, a syrup, a mucoadhesive patch, a gel capsule, gel (a gel which is typically spread/administered over the gingival or buccal area), a liquid drops formulation, a cream and combinations thereof.

When flumazenil is selected as the GABA_{A} receptor antagonist for carrying out the invention, it may be a transmucosal formulation selected from the group consisting of a spray, a tablet, a lozenge, a film, a powder, a syrup, a mucoadhesive patch, a gel capsule, a liquid drops formulation, a cream and a combination thereof.

Sublingual tablets for transmucosal delivery of flumazenil are disclosed herein in Examples 1 and 2. Exemplary sublingual liquid formulations for sublingual delivery of flumazenil are disclosed herein in Examples 11 to 13.

For transmucosal administration, the GABA_{A} receptor antagonist may be combined with one or more inactive ingredients for the preparation of a tablet, packed powder, a spray, edible film strip, soft gel capsule, hard gel capsule, lozenge, cream or troches. For example, in some embodiments, the GABA_{A} receptor antagonist such as flumazenil may be combined with at least one excipient such as fillers, binders, humectants, disintegrating agents, solution retarders, absorption accelerators, wetting agents absorbents, or lubricating agents. According to some embodiments, the antagonist may be combined with one or more of a polyol (e.g., lactose, sucrose, mannitol, or mixtures thereof), an alcohol (e.g., ethanol), and a gum (e.g., acacia and guar), and then formed into a lozenge by conventional methods. In some embodiments, the formulation is a hard, compressed, rapidly dissolving tablet adapted for direct buccal or sublingual dosing. The tablet includes particles made of the GABA_{A} receptor antagonist and a protective material. In some embodiments, these particles are provided in an amount of between about 0.01 and about 75% by weight based on the weight of the tablet (e.g., 1%, 5%, 10%, 15%, 20%, 25%, 30%, 35%, 45%, 50%, 60%, 70%, and 75%). In some embodiments, the tablet may also include a matrix made from a nondirect compression filler, a wicking agent, and a hydrophobic lubricant. In some embodiments, the tablet is adapted to dissolve spontaneously in the mouth of a patient in less than about 60 seconds and, in some cases, in less than about 30 seconds.

Rapidly disintegrating or dissolving buccal tablet formulations are disclosed for example in U.S. Pat. Nos. 7,074,428; 6,923,988; 6,872,405; 6,656,492; 6,589,554; 6,024,981; 5,958,453, and 5,501,861.

In some embodiments, the formulation can be a compressed rapidly dissolving tablet comprising effervescent agents, as disclosed for example in U.S. Pat. No. 6,200,604.

In some embodiments, the GABA_{A} receptor antagonist can be administered transmucosally using an edible film strip, typically comprising water-soluble polymers. In some embodiments, the film is coated and dried utilizing existing coating technology and exhibits instant wettability followed by rapid dissolution/disintegration upon administration in the oral cavity. In some embodiments, such a film can contain a water-soluble polymer or a combination of water-soluble polymers, one or more plasticizers or surfactants, one or more polyalcohols, and flumazenil.

Non-limiting examples of edible films are disclosed for example in U.S. Pat. Nos. 6,923,988; 6,709,671; 6,592,887; 6,284,264; 6,177,096; and 5,948,430.

Powder formulations suitable for transmucosal administration within the nasal cavity are disclosed for example in U.S. Pat. Nos. 6,923,988 and 6,465,626. Gel formulations suitable for transmucosal administration within the buccal and nasal mucosa are disclosed for example in U.S. Pat. Nos. 7,135,190; 5,723,143 and 4,572,832. A porous matrix suitable for transmucosal administration of drugs, including flumazenil, via the sublingual and buccal mucosa is disclosed for example in U.S. Pat. No. 6,932,983.

Spray formulations suitable for administration to the oral or nasal mucosa include aerosol and non-aerosol formulations. Aerosol sprays are typically delivered from a pressurized pack or a nebulizer with the use of a suitable propellant, e.g., dichlorodifluoromethane, trichlorofluoromethane, dichloro-tetrafluoroethane, or carbon dioxide. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch.

Cyclodextrin-drug complexes, including of flumazenil, formulated as sublingual tablets, buccal tablets, nasal spray and nasal drops suitable for transmucosal administration are disclosed in U.S. Pat. No. 6,699,849 and U.S. Patent Application Publication No. 2004/0186075. The disclosure provides formulations of sedative and hypnotic benzodiazepines (alprazolam, triazolam and midazolam) comprising 10% to 14% HPCD, specifically 2-hydroxypropyl-β-cyclodextrin (HPβCD).

Formulations in the form of soft bite gelatin capsules, aerosol and non-aerosol pump sprays, which are suitable for transmucosal administration within the buccal cavity, are disclosed in U.S. Pat. No. 6,977,070. The disclosure provides lingual spray formulations for clozepine, a benzodiazepine, with ethanol, polypropylene glycol (Example 2, Formulation C) or with a propellant (Example 2, Formulation D). The disclosure suggests that the formulations may alternatively contain flumazenil. Formulations comprising the excipients menthol or cyclodextrine are not even mentioned.

Microfluidized nanosuspension pharmaceutical compositions formulated as sprays, aerosols, tablets, capsules, pills, liquids or gels, suitable for transmucosal administration via a buccal route, are disclosed for example in U.S. Pat. No. 6,861,066.

The GABA_{A} receptor antagonist may also be formulated within a mucoadhesive bandage, patch, device or similar preparation that contains the drug and adheres to a mucosal surface. Suitable mucoadhesive patches for transmucosal delivery may comprise a backing, which can be any flexible film that prevents bulk fluid flow and provides a barrier for preventing loss of the drug from the patch. The backing can be any of the conventional materials such as polyethylene, ethyl-vinyl acetate copolymer, polyurethane and the like. In a patch involving a matrix which is not a mucoadhesive, the drug-containing matrix can be coupled with a mucoadhesive component in order that the patch may be retained on the mucosal surface. Suitable configurations include a patch or device wherein the matrix has a smaller periphery than the backing layer such that a portion of the backing layer extends outward from the periphery of the matrix. A mucoadhesive layer covers the outward extending portion of the backing layer such that the underside of the backing layer carries a layer of mucoadhesive around its periphery. The backing and the peripheral ring of mucoadhesive taken together form a reservoir which contains a drug-containing matrix (e.g. a tablet, gel, powder, or a spray). It may be desirable to incorporate a barrier element between the matrix and the mucoadhesive in order to isolate the mucoadhesive from the matrix. The barrier element is preferably substantially impermeable to water and to the mucosal fluids that will be present at intended site of adhesion. A patch or device having such barrier element can be hydrated only through a surface that is in contact with the mucosa, and it is not hydrated via the reservoir. Such patches can be prepared by general methods well known to those skilled in the art.

Suitable mucoadhesives which may be incorporated into transmucosal patches include those well known in the art, such as polyacrylic acids, sodium carboxymethylcellulose, hydroxypropylmethylcellulose and hydroxypropylcellulose.

Transmucosal patches and compositions for preparing such patches, suitable for use in the invention, are disclosed for example in U.S. Pat. Nos. 7,276,246; 7,214,381; 7,198,801; 7,001,609 and 5,750,136, and in International Application Publication No. WO 93/23011.

It may be desirable in some instances to incorporate a mucous membrane penetration enhancer into a transmucosal patch or other transmucosal composition. Suitable penetration enhancers include anionic surfactants (e.g. sodium lauryl sulphate, sodium dodecyl sulphate), cationic surfactants (e.g. palmitoyl DL camitine chloride, cetylpyridinium chloride), nonionic surfactants (e.g. polysorbate 80, polyoxyethylene 9-lauryl ether, glyceryl monolaurate, polyoxyalkylenes, polyoxyethylene 20 cetyl ether), lipids (e.g. oleic acid), bile salts (e.g. sodium glycocholate, sodium taurocholate), and related compounds.

Preparations usable according to the invention may contain additional excipients, also referred to herein as pharmaceutical adjuvants, additives or inert ingredients, such as fillers, bulking agents, binding agents, lubricants, disintegrants, solubilizing vehicles, suspending agents, emulsifying agents, preservatives, flavors, dyes and the like, as is known in the art. Excipients accompany the drug in the formulation of the dosage form in order to facilitate the preparation, patient acceptability and the functioning of the dosage form as a drug delivery system. Excipients should not interfere with the drug's bioavailability, and have no specific pharmacological action in the amount used, but they can alter the pharmacokinetics of the release process and drug absorption.

The GABA_{A} receptor antagonist may be administered by transdermal delivery. The major approaches for transdermal delivery include use of chemical penetration enhancers; physical enhancers, such as ultrasound, iontophoresis, electroporation, magnetophoresis, and microneedles; vesicles; particulate systems, such as those incorporating liposomes, niosomes, transfersomes, microemulsions, or solid lipid nanoparticles, as described for example in Rizwan et al., Recent Pat Drug Deliv Formul., 2009, 3(2):105-24.

Formulations for transdermal administration of the GABA_{A} receptor antagonist may be prepared by mixing the antagonist with suitable pharmaceutical carriers, preservatives, penetration enhancers, and gelling agents to form ointments, emulsions, lotions, solutions, creams, gels, patches or the like, wherein a fixed amount of the preparation is applied onto a certain area of skin.

The term “suitable pharmaceutical carrier” means a non-toxic pharmaceutically acceptable vehicle including, for example, polyethylene glycol, propylene glycol, isopropanol, ethanol, oleic acid, N-methylpyrrolidone, sesame oil, olive oil, wood alcohol ointments, vaseline, and paraffin or a mixture thereof.

The term “penetration enhancer” refers to a chemical compound or combination thereof that facilitates absorption of an active pharmaceutical ingredient across and through a dermal surface (see for example, Karande et al., Proc Natl. Acad. Sci. USA Mar. 29, 2005, 102(13):4688-4693).

Suitable penetration enhancers include, for example, saturated and unsaturated fatty acids and their esters, alcohols, monoglycerides, diethanolamines, N,N-dimethylamines such as linolenic acid, linolenyl alcohol, oleic acid, oleyl alcohol, stearic acid, stearyl alcohol, palmitic acid, palmityl alcohol, myristic acid, myristyl alcohol, 1-dodecanol, 2-dodecanol, lauric acid, decanol, capric acid, octanol, caprylic acid, 1-dodecylazacycloheptan-2-one, ethyl caprylate, isopropyl myristate, hexamethylene lauramide, hexamethylene palmitate, capryl alcohol, decyl methyl sulfoxide, dimethyl sulfoxide, salicylic acid and its derivatives, N,N-diethyl-m-toluamide, 1-substituted azacycloalkan-2-ones, propylene glycol, polyethylene and glycol monolaurate. Any compound compatible with the selected GABA_{A} receptor antagonist, in particular flumazenil, and that has transdermal permeation enhancing activity may be selected.

U.S. Pat. Nos. 4,006,218; 3,551,154; and 3,472,931 describe the use of dimethylsulfoxide, dimethyl formamide, and N,N-dimethylacetamide as penetration enhancers. U.S. Pat. No. 4,973,468 describes the use of combinations of diethylene glycol monoethyl or monomethyl ether with propylene glycol monolaurate and methyl laurate as penetration enhancers. U.S. Pat. No. 4,820,720 describes a dual enhancer consisting of glycerol monolaurate and ethanol. U.S. Pat. No. 5,006,342 lists numerous enhancers for transdermal drug administration consisting of fatty acid esters or fatty alcohol ethers of C2 to C4 alkanediols, where each fatty acid/alcohol portion of the ester/ether is of about 8 to 22 carbon atoms. U.S. Pat. No. 4,863,970 shows penetration-enhancing compositions comprising an active permeant contained in a penetration-enhancing vehicle containing one or more cell-envelope disordering compounds such as oleic acid, oleyl alcohol, and glycerol esters of oleic acid; or a C2 or C3 alkanol. Other disclosed penetration enhances include menthol (U.S. Pat. No. 4,933,184); vegetable oil (U.S. Pat. No. 5,229,130), and eucalyptol (U.S. Pat. No. 4,440,777).

Creams for transdermal delivery of flumazenil are disclosed herein in Examples 7 and 8. Suitable gelling agents include, for example, hydroxy methyl cellulose, hydroxypropyl cellulose, tragacanth, sodium alginate, gelatin, methylcellulose, sodium carboxymethylcellulose, and polyvinyl alcohols.

Suitable preservatives include, for example, parabens, benzoic acid, and chlorocresol. Antioxidants can also be included, for example, ascorbyl palmirate, butylated hydroxyanisole, butylated hydroxytoluene, potassium sorbate, sodium bisulfate, sorbic acid, propyl gallate and sodium metabisulfite.

In some embodiments, the antagonist is administered by a transdermal patch. Transdermal drug delivery using patch technology is based on the ability to hold an active ingredient in constant contact with the epidermis for a substantial period of time, such that drug molecules, held in such a state, will eventually enters the bloodstream (for reviews, see for example, Ball et al., Am J Health Syst Pharm. 2008 Jul. 15; 65(14):1337-46). These delivery systems comprise a patch with an active drug ingredient incorporated therein, and an adhesive for attachment to the skin. Exemplary patch technologies are available from Ciba-Geigy Corporation and Alza Corporation, and may be readily adapted for use with a GABAA receptor antagonist, such as flumazenil, for carrying out the invention.

Adhesives for making transdermal patches for use in the methods described herein include polyisobutylene, silicone based adhesives, and acrylic polymers. The adhesive polymers can be mixed with other excipients such as waxes and oils (e.g., mineral oil). A protective liner can be placed in contact with the adhesive layer to protect against drug release from the patch prior to application. Liners for use with the transdermal patches described herein include, for example, polyethylene terephthalate film, polyester membrane, and polycarbonate film.

The backing membrane of the transdermal patch for use with the methods described herein constitutes the top face surface of the transdermal patch. It may be made of a single layer or film of polymer, or be a laminate of one or more polymer layers and metal foil. Examples of polymers suitable for use in making backing films include, for example, polyester films, polyolefins, ethyl vinyl acetate, polypropylene, polyethylene, polyurethanes, polyvinyl alcohols, polyvinyl chlorides, polyamides, ethylene ethylacrylate copolymer, vinyl acetate vinyl chloride copolymer, cellulose acetate and ethyl cellulose. Drug-impermeable, elastic backing materials may be selected from polyethylene tereplithalate, polyurethane, ethylene vinyl acetate copolymer, plasticized polyvinylchloride, and woven and non-woven fabric.

In particular embodiments, the administration rate of the drug is 0.1-1000 μg/h through a skin area of about 2-90 cm² (e.g., 10-30 cm²).

The amount of drug delivered into the skin can be controlled by a number of factors including skin patch size, degree of drug loading, the use of rate controlling membranes, permeation enhancers, and the like.

Transdermal patches are disclosed for example in U.S. Pat. Nos. 6,541,021; 5,591,767 and 5,124,157.

Also useful for the invention are transdermal patches which incorporate microneedles, as disclosed for example in U.S. Patent Application Publication Nos. 2009/0250176; 2009/0118672; 2009/0043270; and 2008/012543.

Transdermal administration may also comprise use of ethosomal vesicles, as described for example in U.S. Pat. Nos. 5,716,638 and 5,540,934.

In particular embodiments, administering for providing transdermal delivery comprises use of a means selected from the group consisting of a patch, an iontophoretic delivery device, a timed-release formulation, ethosomes, liposomes, microneedles and a combination thereof. In particular embodiments, the patch comprises a timed-release formulation. In particular embodiments, the patch comprises at least one of ethosomes, liposomes and microneedles.

In some embodiments, the transmucosal and/or the transdermal formulation may be a timed-release or controlled release formulation. The transmucosal or transdermal formulation described herein may be formulated so as to provide slow or controlled release of the GABA_{A} receptor antagonist using, for example, hydropropylmethyl cellulose in varying proportions to provide the desired release profile. Other polymer matrices, gels, permeable membranes, osmotic systems, multilayer coatings, microparticles, liposomes and/or microspheres may be used provide the desired release profile.

In general, a timed-release preparation is a pharmaceutical composition in which delivery of the active ingredient is withheld or delayed following administration of the dosage form. Accordingly, there is a lag time (“delay”) between the time point at which the patch is applied to the skin and time point at which the active ingredient becomes bioavailable and/or exerts a pharmacological effect.

In general, a controlled release preparation is a pharmaceutical composition capable of releasing the active ingredient at the required rate to maintain constant pharmacological activity for a desirable period of time. Such dosage forms provide a supply of a drug to the body during a predetermined period of time and thus maintain drug levels in the therapeutic range for longer periods of time than conventional non-controlled formulations.

U.S. Pat. No. 5,120,548 discloses a controlled-release drug delivery device comprised of swellable polymers. U.S. Pat. No. 5,073,543 describes controlled-release formulations containing a trophic factor entrapped by a ganglioside-liposome vehicle. U.S. Pat. No. 5,639,476 discloses a stable solid controlled-release formulation having a coating derived from an aqueous dispersion of a hydrophobic acrylic polymer. Biodegradable microparticles are known for use in controlled-release formulations. U.S. Pat. No. 5,733,566 describes the use of polymeric microparticles that release antiparasitic compositions.

The controlled-release of the active ingredient may be stimulated by various inducers, for example, pH, temperature, enzymes, water, or other physiological conditions or compounds. Various mechanisms of drug release exist. For example, in one embodiment, the controlled-release component may swell and form porous openings large enough to release the antagonist after administration to a patient. The term “controlled-release component” means a compound or compounds, such as polymers, polymer matrices, gels, permeable membranes, liposomes and/or microspheres that facilitate the controlled-release of the active ingredient in the pharmaceutical composition. In another embodiment, the controlled-release component is biodegradable, induced by exposure to the aqueous environment, pH, temperature, or enzymes in the body. Means for controlled release of the formulation of the invention also include iontophoresis.

In some embodiments, administration of the antagonist may be performed using an implantable device, for example, an implantable, self-regulating mechanochemical subdermal pump. In some embodiments, the device may administer the antagonist on a set dosage program. In some embodiments, the device may administer the antagonist on demand as determined by the subject. In some embodiments, the device may administer the antagonist on a constant release profile. In some embodiments, the device may administer the antagonist automatically. These devices are known in the art for the treatment of other disorders, for example, diabetes. Non-limiting examples of various embodiments of this mode of administration are detailed in U.S. Pat. Nos. 7,544,190; 7,162,297; 6,852,104; 5,324,518; and 5,062,841. Furthermore, the device may be one which provides iontophoretic transdermal delivery, as described for example in U.S. Pat. Nos. 7,574,256; 7,031,768 and 5,320,731. In some embodiments, a transmucosal administration of an GABA_{A} receptor antagonist may be combined with transdermal administration of the same or another GABA_{A} receptor antagonist. Without being bound by theory, such a delivery mechanism may be useful for nocturnal application to assist the subject with morning wakefulness or assist night shift workers to improved wakefulness.

In particular embodiments, administering the GABA_{A} receptor antagonist may comprise use of both a transmucosal route of delivery and a transdermal route of delivery. For example, delivery of the antagonist may be initiated by use of a transdermal patch, and the subject may then use a sublingual spray to achieve the desired effect. Alternately, the delivery may be initiated by use of a transmucosal dosage form, followed by use of a transdermal dosage form.

### Dosages of GABA_{A} Receptor Antagonists

The specific dose of a GABA_{A} receptor antagonist required to obtain therapeutic benefit in the methods of treatment described herein will, usually be determined by the particular circumstances of the individual patient including the size, weight, age, and sex of the subject, the nature and stage of the disorder being treated, the aggressiveness of the disorder, and the route of administration of the compound.

For transmucosal administration (e.g., sublingual administration), for example, a daily dosage of flumazenil, for example, can range from about 0.2 mg to about 10 mg (e.g., about 0.5 mg to about 5 mg; about 1 mg to about 3 mg; about 1.5 mg to about 4 mg; about 2 mg to about 6 mg; about 1.25 mg to about 8 mg; and about 4 mg to about 10 mg). In particular embodiments, the flumazenil is administered in an amount in the range of about 0.2 to about mg over a 24 hour period. In particular embodiments, the flumazenil is administered in an amount of about 4 mg over a 24 hour period. In particular embodiments, the flumazenil is administered in an amount in the range of about 0.2 to about 1.0 mg per dose. In particular embodiments, the flumazenil is administered in an amount of about 0.2 mg per dose. In particular embodiments, the flumazenil is administered in an amount of about 0.4 mg per dose. In particular embodiments, the flumazenil is administered in an amount of about 0.8 mg per dose. In particular embodiments, multiple doses of flumazenil are administered. In particular embodiments, the doses of flumazenil are administered every 30 minutes. In particular embodiments, flumazenil in an amount of 0.2 mg per dose is administered every 30 minutes.

Higher or lower doses are also contemplated, as it may be necessary to use dosages outside these ranges in some cases while maintaining a positive risk benefit therapeutic profile.

The transmucosal formulation can be administered in one single dose or the daily dose may be divided, such as being divided equally into two to six times per day daily dosing. In some embodiments, the transmucosal formulation is administered at least twice daily. In some embodiments, the transmucosal formulation is administered at least three times daily. In some embodiments, the transmucosal formulation is administered about every one to six hours (e.g., about every one hour; about every two hours; about every three hours; about every three and a half hours; about every four hours; about every five hours; and about every six hours). In some embodiments, the transmucosal formulation is administered by the subject as needed, e.g., patient controlled titration to a desired end effect (e.g., wakefulness or reduced sleepiness), for example every 10 minutes or increasing dose. In some embodiments the first dose is 0.2 mg and if an insufficient effect is achieved, the subject can administer a subsequent dose of the same amount or a higher dose, e.g. of 0.4 mg. In some embodiments after having partial response the user can administer another dose after 10 minutes.

In addition, as the GABA_{A} receptor antagonist is expected to have reduced effect after 20-40 minutes, an additional dose may be administered when the effect is decreasing. In some embodiments, GABA_{A} receptor antagonist is administered upon awakening, and a subsequent dose is administered 20 minutes thereafter.

A transmucosal formulation may be formulated in a unit dosage form, each dosage containing from about 0.2 to about 10 mg of the antagonist, e.g., flumazenil, per unit dosage (e.g., about 0.2 mg to about 5 mg; about 0.5 mg to about 5 mg; about 1 mg to about 10 mg; about 1.5 mg to about 8 mg; about 2 mg to about 7 mg; about 3 mg to about 6 mg; about 4 mg to about 8 mg; about 5 mg to about 10 mg; about 6 mg to about 8 mg; and about 8 mg to about 10 mg. In some embodiments, each dosage can contain about 5 to about 10 mg of the GABA_{A} receptor antagonist per unit dosage. In some embodiments, each dosage contains about 6 mg of the GABA_{A} receptor antagonist. The term “unit dosage form” refers to physically discrete units suitable as a unitary dosage for human subjects and other mammals, each unit containing a predetermined quantity of active material calculated to produce the desired therapeutic effect, in association with a suitable pharmaceutical excipient. A preprogrammed setting would be able, for example, to limit the minimal time between doses and/or the maximal delivered dose per unit of time (e.g. up to 3 mg of flumazenil per hour) for both transdermal and the transmucosal formulations and delivery systems.

For transdermal administration, for example, a daily dosage of flumazenil can range from about 0.2 mg to about 10 mg (e.g., about 0.2 mg to about 2 mg; about 0.5 mg to about 5 mg; about 1 mg to about 3 mg; about 1.5 mg to about 4 mg; about 2 mg to about 6 mg; about 1.25 mg to about 8 mg; and about 4 mg to about 10 mg). In some embodiments, a daily dosage of transdermal flumazenil can range from about 1 mg to about 5 mg. In some embodiments, a daily dosage of transdermal flumazenil can be about 1.5 mg. In some embodiments, a daily dosage of transdermal flumazenil can be about 2 mg. In some embodiments, a daily dosage of transdermal flumazenil can be about 3 mg. Higher or lower doses are also contemplated as it may be necessary to use dosages outside these ranges in some cases.

The transdermal formulation can be administered in one single dosage or the daily dosage may be divided, such as being divided equally into two to six times per day daily dosing. In some embodiments the transdermal formulation is formulated to a concentration of about 0.2 mg to about 10 mg per mL (e.g., about 0.5 mg to about 8 mg per mL; about 1 mg to about 6 mg per mL; about 1.5 mg to about 5 mg per mL; about 3 mg to about 7 mg per mL; about 4 mg to about 10 mg per mL; and about 4 mg to about 8 mg per mL). In some embodiments, the transdermal formulation is formulated to a concentration of about 4 mg per mL. In some embodiments, the transdermal formulation is administered once daily (e.g., before bed). In some embodiments, the transdermal formulation is administered at least twice daily. In some embodiments, the transdermal formulation is administered about every eight to about twenty-four hours (e.g., about every eight hours; about every ten hours; about every twelve hours; about every sixteen hours; about every twenty hours; about every twenty-two hours; and about every twenty-four hours).

A transdermal formulation may be formulated in a unit dosage form, each dosage containing from about 0.2 to about 10 mg of flumazenil per unit dosage (e.g., about 0.2 mg to about 2 mg; about 0.5 mg to about 8 mg; about 1 mg to about 5 mg; about 1.5 mg to about 4 mg; about 2 mg to about 6 mg; about 3 mg to about 7 mg; about 4 mg to about 8 mg; and about 5 mg to about 10 mg). In some embodiments, each dosage can contain about 1 to about 4 mg of flumazenil per unit dosage. In some embodiments, each dosage contains about 2 mg of flumazenil. The term “unit dosage form” refers to physically discrete units suitable as a unitary dosage for human subjects and other mammals, each unit containing a predetermined quantity of active material calculated to produce the desired therapeutic effect, in association with a suitable pharmaceutical excipient.

The components used to formulate the pharmaceutical compositions described above are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The GABA_{A} receptor antagonist can be administered in combination with other agents, particularly, an agent selected from the group consisting of: energizers, invigorants, nervous system stimulants, and psycostimulants, including but not restricted to amphetamines, methylphenidate, venlafaxine, nefazodone, sodium oxybate, adrafinil, modafinil, armodafinil, phentermine, pemoline, adrenaline, methylxantines, theobromine, caffeine and any medication/substance that will increase blood/brain orexin levels.

In one embodiment, the GABA_{A} receptor antagonist is administered with a wakefulness promoting agent. In some embodiments, the wakefulness promoting agent is selected from modafinil, armodafinil, adrafinil, methylphenidate, venlafaxine, nefazodone, sodium oxybate, phentermine, pemoline, adrenaline, methylxantines, theobromine, caffeine, a herbal product such as ginseng, and a combination thereof. In some embodiments, the wakefulness promoting agent is modafmil. The wakefulness promoting agent can be administered in an amount less than about 600 mg per day (e.g., less than about 100 mg per day; less than about 200 mg per day; less than about 300 mg per day; less than about 400 mg per day; less than about 500 mg per day; and less than about 600 mg per day). The specific dose of a wakefulness promoting agent required to obtain therapeutic benefit in the methods of treatment described herein will usually be determined by the particular circumstances of the individual subject including the size, weight, age, and sex of the subject, the nature and stage of the disorder being treated, the severity of the disorder, and the route of administration of the compound. In some embodiments, the wakefulness promoting agent can be administered twice daily. In some embodiments, the wakefulness promoting agent can be administered in an amount of 5 mg per BMI unit. In some embodiments, the wakefulness promoting agent can be administered in an amount of 100 mg per dose.

The mode of use of the GABA_{A} receptor antagonist can be sporadic, chronic or alternating, unrelated to the pattern of use of the sleep drugs. In some embodiments, the sleep drug administered every evening while the GABA_{A} receptor antagonist can be administered every other day, or in some embodiments every day, or only when as desired by the subject.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1

### Formulation of Flumazenil as Tablet for Sublingual Dosing

TABLE 1

| Ingredient | Amount | |
|---|---|---|
| Flumazenil | 0.3 | grams |
| Tablet triturate base (20%/80% powder) | 4.7 | grams |
| Tablet triturate excipient (flavorless) | 2 | milliliters |
| Flavor | 4 | drops |
| Stevia concentrate (250 mg/mL) | 2 | drops |

The preparation of the tablet triturate base is described in Example 3. The ingredients are combined and mixed to form a thick paste. After the thick paste is formed, a flavor is added. The flavor added is selected from the following: a) 2 drops lemon, 1 drop marshmallow, 4 mg yellow color b) 2 drops creme de mint, 4 mg green color c) 2 drops tangerine, 1 drop marshmallow, 4 mg orange. The preparation is sufficient to provide 50 tablets.

### Example 2

### Formulation of Flumazenil as Tablet for Sublingual Dosing

TABLE 2

| Ingredient | Amount | |
|---|---|---|
| Flumazenil | 0.6 | grams |
| Tablet triturate base (20%/80% powder) | 9.4 | grams |
| Tablet triturate excipient (flavorless) | 4 | milliliters |
| Flavor | 8 | drops |
| Stevia concentrate (250 mg/mL) | 4 | drops |

The preparation of the tablet triturate base is described in Example 3. The ingredients are combined and mixed to form a thick paste. After the thick paste is formed, a flavor is added. The flavor added is selected from the following: a) 2 drops lemon, 1 drop marshmallow, 4 mg yellow color b) 2 drops creme de mint, 4 mg green color c) 2 drops tangerine, 1 drop marshmallow, 4 mg orange d) 5 drops cherry, 2 drops vanilla, 4 mg red color. The formulation is sufficient to provide 100 tablets.

### Example 3

### Formulation of Tablet Triturate Base 20%/80% Powder

TABLE 3

| Ingredient | Amount |
|---|---|
| Sucrose powdered (confectioners) | 20 grams |
| Lactose monohydrate (hydrous) | 80 grams |

The sucrose and lactose monohydrate are sieved through 120 or smaller mesh. After adding the active ingredient (e.g., flumazenil), the mixture is wetted with an excipient of 40% distilled water and 60% alcohol. The formulation is sufficient to provide 100 grams of tablet triturate base 20%/80% powder.

### Example 4

### Formulation of Stevia Concentrate Solution (250 mg/ml)

TABLE 4

| Ingredient | Amount | |
|---|---|---|
| Stevia powder extract | 25 | grams |
| Sodium benzoate | 0.6 | grams |
| Water preserved liquid | 100 | milliliters |

The preparation of the water preserved liquid is described in Example 5. The Stevia powder and sodium benzoate are dissolved in the water preserved liquid. The mixture is warmed to aid in dissolution. The formulation is sufficient to prepare 100 mL of Stevia concentrate solution.

### Example 5

### Formulation of Water Preserved (Paraben) Liquid

TABLE 5

| Ingredient | Amount | |
|---|---|---|
| Water preserved concentrate liquid | 10 | milliliters |
| Water distilled | 3780 | milliliters |

The preparation of the water preserved concentrate liquid formulation is described in Example 6. The liquids are mixed to prepare the water preserved (paraben) liquid.

### Example 6

### Formulation of Water Preserved Concentrate Liquid

TABLE 6

| Ingredient | Amount | |
|---|---|---|
| Methylparaben | 19 | grams |
| Propylparaben | 9.6 | grams |
| Propylene glycol USP | 100 | milliliters |

The ingredients are mixed together and stirred until the methylparaben and propylparaben NF are completely dissolved.

### Example 7

### Formulation of Flumazenil Cream for Transdermal Dosing

TABLE 7

| Ingredient | Amount | |
|---|---|---|
| Flumazenil | 0.04 | grams |
| Propylene glycol USP | 0.1 | milligrams |
| Food color, pink (powder) | 0.03 | mg |
| VersaBase ® cream | 10 | grams |

The ingredients are combined and mixed. The formulation is sufficient to provide 10 milliliters of cream.

### Example 8

### Formulation of Flumazenil Cream for Transdermal Dosing

TABLE 8

| Ingredient | Amount | |
|---|---|---|
| Flumazenil | 0.25 | grams |
| Propylene glycol USP | 0.25 | milligrams |
| Food color, pink (powder) | 0.0075 | mg |
| VersaBase ® cream | 25 | grams |

The ingredients are combined and mixed. The formulation is sufficient to provide 25 milliliters of cream.

### Example 9

### Preparation and Use of a Transdermal Patch for Inducing Sleep and Maintaining Daytime Alertness with Reduced Side Effects

The drugs for incorporation into the transdermal patch are denoted as Component A, Component B, Component C and Component D.

Component A is a sleep drug which provides fast sleep onset, while component B is a sleep drug which maintains sleep. Component C is a GABA_{A} receptor antagonist which counteracts the effect of Components A and B. Component D is a wakefulness promoting agent for sustaining alertness during waking hours. The transdermal patch may contain only one of Components A and B. Component D is also optional.

Components A and B may be selected from the group comprised of tonics, calmatives, hypnotics, muscle relaxants, sedative anti anxiety agents, anti-depressant (e.g. tri-cyclic), anti insomnia agents, tranquilizers, neutral and herbal sedative materials, hormones, hormones and medications such as benzodiazepines (e.g alprazolam, bromazepam, clonazepam, clotiazepam, cloxazolam, diazepam, estazolam, etizolam, fludiazepam, flunitrazepam, flurazepam, halazepam, haloxazolam, lorazepam, medazepam, midazolam, nimetazepam, nitrazepam, olanzapine, oxazepam, quazepam, temazepam, and triazolam and derivates thereof); non-benzodiazepines such as adipiplon (NG-2-73), agomelatine, almoxerant (ACT-078573), brotizolam, diphenhydramine, divaplon, doxepin, eplivanserin (SR 46349), doxylamine succinate, eszopiclone, indiplon, ocinaplon, pagoclone, pazinaclone, pruvanserin (EMD 281014), suproclone, suriclone, L-tryptophan, 5-hydroxy-L-tryptophan, melatonin, melatonin receptor agonists, such as VEC-162 and PD-6735, muramyl dipeptide, ramelteon, sleep-promoting substance, uridine, volinanserin (M-100907), zaleplon, zolpidem, APD125, ACP-103, PD 200-390, HY10275, GW649863, EVT-201, L-tryptophan, 5-hydroxy-L-tryptopan, melatonin and/or melatonin agonists, sleep promoter factor, orexin antagonists, 5-HT agonists and derivatives thereof.

Component C is a GABA_{A} receptor antagonist, for example: flumazenil, clarithromycin, picrotoxin, bicuculline, cicutoxin or oenanthotoxin, and is currently preferred embodiment, is flumazenil.

Component D is any agent selected from the group comprised of pharmaceutically active energizers, invigorants, nervous system stimulants, and psycostimulants, including but not restricted to amphetamines, methylphenidate, venlafaxine, nefazodone, sodium oxybate, adrafinil, modafinil, armodafinil, phentermine, pemoline, adrenaline, methylxantines, theobromine, caffeine and any medication/substance that will increase blood/brain orexin levels.

In a particular embodiment, the transdermal patch comprises triazolam as Component A, temazepam as Component B, flumazenil as Component C and methylphenidate as Component D.

The patch includes several geometrically configured sections or regions that provide a sequence of drug delivery according to the therapeutic effect of each Component. Each region is designed to deliver Component A of quantity Qa for a period of time Ta (delivery rate Ra), optionally with a delay Da; Component B of quantity Qb for a period of time Tb, optionally with a delay Db, and so on.

One embodiment of the patch has N sections (N being the number of sections corresponding to the number of components) as shown in FIG. 2. Each section has an area Ai (i is a number of section from 1 to N) that is proportional to the rate of the delivery (Ra) that is required for the component present in the section.

Each section Ai has several layers that provide controllable drug delivery. FIG. 3 shows a representative cross-sectional view of a section Ai. Layer 21 is a top layer that protects the drug and the label. Capsule 22 is a pressure rupturable compartment that contains the drug in liquid form. Capsule 22 prevents leakage of the drug during storage. Upon exertion of pressure on the patch, capsule 22 opens and the drug liquid is capable of moving to the layer 23 which is diffusion membrane designed to delay the drug delivery across the skin for a time Di at a rate Ri. Adhesive layer 24 and removable sealing tape 25 are designed to adhere to the skin and provide adhesion and enable drug penetration to the skin. Layer 25 may optionally include skin penetration enhancers. Each section Ai of the patch may vary with respect to capsule 22 size and diffusion layer 23 to provide the required delay and delivery rate of the Component provided in the section. One or more additional membranes for the control of delivery may be included in a particular section to ensure the required dosage at the desired time.

The application of the patch involves removal of the sealing tape 25, adhering the patch onto the skin and exerting downward pressure onto the top of the patch in order to activate (i.e. rupture) the capsules so that the drug liquid can access the skin via the diffusion membranes. This is only one possible configuration of the patch design. Several other variations are possible, including a configuration of one section positioned on top of the other so that drugs diffuse in sequence across the skin.

In one embodiment, the patch provides: a first sleep drug as Component A at a rate which induces sleep within up to 30-60 minutes; a second sleep drug as Component B, the delivery of which is delayed for a lag time of 1 to 2 hours and released over a period of 2 to 4 hours for sustaining the sleep; flumazenil as Component C the delivery of which is delayed for a lag time of 5 to 7 hours and released over a period of 20 minutes for awakening the patient.

The determination of the geometry and configuration of each patch section so as to provide the required delivery parameters for each specific drug component, is within the ability of one of average skill in the art.

### Example 10

### Iontophoretic Drug Delivery Device

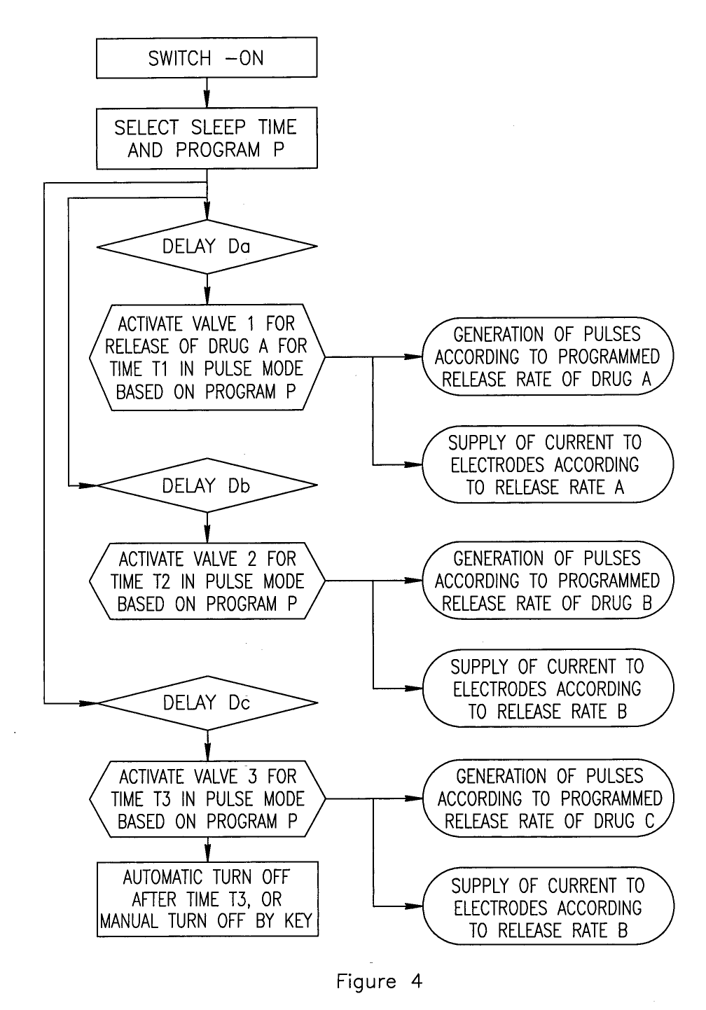

An electronic transdermal drug delivery may be used for drug delivery by micro-valves or electronically controlled membranes and enhance the skin penetration by electric current in the process known as iontophoresis. An advantage of this technique is that the device may be programmed for exact adjustment of delivery of each component. The system includes a drug capsule coupled to an electronic micro-valve or micro-pump that controls the drug delivery to the patch that enables the drug penetration through the skin. One embodiment is shown in FIG. 4. Capsule 31 contains one drug component that is coupled by a tube to a micro-valve 33 and/or micro-pump 36 that is controlled by electronic control unit 32 (preferably ASIC with a battery). By activation of the micro-valve 33 and/or micro-pump 36, the drug components penetrate into the diffusion membrane or matrix 34 and diffuse onto adhesion layer 39 that is attached to the skin surface. The electronic unit is switched on and optionally programmed by user interface 38 via keys. The electronic unit controls several micro-valves 33 and/or micro-pumps 36 and capsules 37 and 31. Optionally the electronic unit 32 controls and provides electric current to the electrodes 35 that provide ion transport and iontophoresis. The user is able to set the starting time of the drug delivery and the total sleeping time that will be used by the electronic unit to release the drug components according to the corresponding program. Optionally the user interface includes controls that enable adjustment of a personal drug dosage and delivery of a special combination of the respective components, for example the user can add a dose by pressing a button, or for example can initiate the delivery by pushing a button. Alternatively, the device is preprogrammed for dosage and timing of the components according to the total sleep time only. The device can be disposable or multiple use. In the case of a multiple-use device, it has on-off switch (key), and any of the adhesive layer 39, diffusion membrane 34, and electronic control unit 32 may be disposable and replaceable. An alternative design for a reusable device includes an electronic unit 32 as a separate module that connects via mechanical and electric interface (connector) to disposable (or limited use) patch module. The device can be pre-programmed as to limit the maximal total dose delivered, and/or the maximal delivered units per unit of time or preprogrammed for the minimal time between doses.

The algorithm of operation of the electronic circle includes turning on, getting input of the sleep time and optionally the program type that the device should perform. The system (ASIC) calculates the delays Di, times Ti and delivery rates Ri for each drug component based on the program parameters and input sleep time. The Ri is translated to number of electrical pulses per minute that activate the micro-pump or micro valve. The current to electrodes is also calculated based on Ri.

An example of a program is:

a) delivering 0.125 mg of Drug A for 20 minutes at device onset, Ra=0.6 mg/h;
b) delivering 2 mg/h of Drug B after 2 hours from onset, for 2 hours; and
c) delivering Drug C 0.2 mg during 20 minutes Rc=1 mg/h, 7 hours from the onset of patch activation.

Providing that the input is 7 hours of sleep, the system calculates the delays for activation of each of drug components A, B and C and the respective delivery rates Ri, by using simple formulas. The rates are translated into pulse sequence rate based on constant factor of the delivery system or function.

In an optional embodiment, the sensor and signal processing module 310 is connected to 32 and provides input on sleep quality and/or sleep staging for optimal control of sedation. When the sleep quality is poor, as detected by body movement, the control unit 32 increases the delivery rate of the drug. In a case of skin temperature sensor, the decrease of skin temperature is related to NREM sleep and slow wave sleep (SWS). In normal sleep, after 60-80 minutes of NREM and SWS the drug delivery rate can be changed to enable REM sleep. Thus the sensor enables adaptive control of drug delivery.

### Example 11

### Flumazenil Formulation for Sublingual Administration

TABLE 9A
Formulation A1 (without preservatives)

| Ingredient | % w/w | Function |
|---|---|---|
| Flumazenil | 0.4 | API |
| Ethanol | 20 | Solubilizing Agent |
| Propylene glycol | 20 | Solubilizing Agent |
| 30% HPCD (hydroxypropyl-β- | 59.5 | Solubilizing Agent |
| cyclodextrin) in citric buffer | | |
| 10 mM pH 4.0 | | |
| Menthol in EtOH (1:1 w/w) | 0.1 | Penetration Enhancer |

Formulation preparation:
1. Citric buffer 10 mM pH 4.0 is prepared according to the following composition: Citric acid anhydrous—0.1199% w/v and sodium citrate dihydrate—0.1105% w/v is dissolved in water and the pH is adjusted to 4.0±0.2 if needed.
2. HPCD 30% w/w in Citric buffer 10 mM pH 4.0 solution is prepared.
3. Menthol in EtOH solution is prepared by dissolving Menthol in Ethanol absolute in a ratio of 1:1 w/w.
4. Flumazenil is weighed and EtOH, Menthol solution, propylene glycol and HPCD 30% in Citric buffer 10 mM pH 4.0 solution is added according to Table 9A. The mixture is stirred until a clear solution is obtained.

TABLE 9B
Formulation A2

| Ingredient | % (w/w) |
|---|---|
| Flumazenil | 0.4 |
| Ethanol Absolute | 20.05 |
| Propylene glycol | 20.0 |
| Kleptose HPB oral (hydroxypropyl-β- | 17.85 |
| cyclodextrin-HPCD | |
| Citric acid anhydrous | 0.05 |
| Sodium citrate dihydrate | 0.05 |
| Menthol in EtOH (1:1 w/w) | 0.05 |
| Water for Injection | 41.55 |

### Example 12

### Flumazenil Formulation for Sublingual Administration

TABLE 10
Formulations B and C (containing benzyl alcohol)

| | Formu- | Formu- | |
|---|---|---|---|
| | lation B | lation C | |
| Ingredient | % w/w | % w/w | Function |
| Flumazenil | 0.4 | 0.4 | API |
| Ethanol | 20 | 20 | Solubilizing Agent |
| Propylene glycol | 20 | 20 | Solubilizing Agent |
| 30% HPCD (hydroxypropyl- | 57.5 | 58.5 | Solubilizing Agent |
| β-cyclodextrin) in citric | | | |
| buffer; 10 mM pH 4.0 | | | |
| Menthol in EtOH (1:1 w/w) | 0.1 | 0.1 | Penetration Enhancer |
| Benzyl alcohol | 2.0 | 1.0 | Preservative |

Formulation preparation: Formulations B and C are prepared as described above for formulation A, with the addition of benzyl alcohol according to Table 10.

### Example 13

### Flumazenil Formulation for Sublingual Administration

TABLE 11
Formulations D and E (containing methyl and propyl parabens)

| | Formu- | Formu- | |
|---|---|---|---|
| | lation D | lation E | |
| Ingredient | % w/w | % w/w | Function |
| Flumazenil | 0.4 | 0.4 | API |
| Ethanol | 10 | 15 | Solubilizing Agent |
| Propylene glycol | 20 | 20 | Solubilizing Agent |
| 30% HPCD (hydroxypropyl- | 59.3 | 59.4 | Solubilizing Agent |
| β-cyclodextrin) in citric | | | |
| buffer | | | |
| 10 mM pH 4.0 | | | |
| Menthol in EtOH (1:1 w/w) | 0.1 | 0.1 | Penetration Enhancer |
| Propylparaben/Methylparaben | 10.2 | 5.1 | Preservative |
| in EtOH 0.02:0.18:10 w/w/w | | | |

Formulation preparation:
1. Citric buffer 10 mM pH 4.0 is prepared according to the following composition: Citric acid anhydrous—0.1199% w/v and sodium citrate dihydrate—0.1105% w/v is dissolved in water and the pH is adjusted to 4.0±0.2 if needed.
2. HPCD 30% w/w in Citric buffer 10 mM pH 4.0 solution is prepared.
3. Menthol in EtOH solution is prepared by dissolving Menthol in Ethanol absolute in a ratio of 1:1 w/w.
4. Propylparaben and Methylparaben are dissolved in Ethanol in a ration of 0.02:0.18:10 w/w/w.
5. Flumazenil is weighed and EtOH, parabens solutions, Menthol solution, propylene glycol and HPCD 30% in Citric buffer 10 mM pH 4.0 solution is added according to Table 11. The mixture is stirred until a clear solution is obtained.

### Example 14

### Flumazenil Formulation for Sublingual Administration

TABLE 12
Formulations F and G - no preservatives

| | Formu- | Formu- | |
|---|---|---|---|
| | lation F | lation G | |
| Ingredient | % w/w | % w/w | Function |
| Flumazenil | 0.4 | 0.4 | API |
| Ethanol | 25 | 20 | Solubilizing |
| | | | Agent |
| Propylene glycol | 25 | 20 | Solubilizing |
| | | | Agent |
| Citric buffer | 49.6 | | Buffer |
| 10 mM pH 4.0 | | | |
| 30% HPCD (hydroxypropyl-β- | | 59.6 | Solubilizing |
| cyclodextrin) in citric buffer | | | Agent |
| 10 mM pH 4.0 | | | |
| Appearance | C | C | |
| pH after preparation | 4.93 | 5.13 | |
| pH after 1 week storage at 25° C. | 5.07 | 5.14 | |
| pH after 3 week storage at | 5.04 | 5.22 | |
| ambient temperature | | | |

C = clear

Formulations F and G were prepared in accordance with the method described above for Example 11. 100 μl sublingual formulation contains 0.4 mg flumazenil.

### Example 15

### Placebo Formulations

TABLE 13
Placebo formulations for formulations A′-G′

| | Placebo for Formulation No. | | | | | | |
|---|---|---|---|---|---|---|---|
| | A′ | B′ | C′ | D′ | E′ | F′ | G′ |
| Ethanol | 20 | 20 | 20 | 10 | 15 | 25 | 20 |
| Propylene glycol | 20 | 20 | 20 | 20 | 20 | 52 | 20 |
| Citric buffer 10 mM pH 4.0 | | | | | | 50 | |
| 30% HPCD in citric buffer | 59.9 | 57.9 | 58.9 | 59.7 | 59.8 | | 60 |
| 10 mM pH 4.0 | | | | | | | |
| Menthol in EtOH (1:1 w/w) | 0.1 | 0.1 | 0.1 | 0.1 | 0.1 | | |
| Benzyl alcohol | | 2.0 | 1.0 | | | | |
| Propylparaben/ | | | | 10.2 | 5.1 | | |
| Methylparaben in EtOH | | | | | | | |
| 0.02:0.18:10 w/w/w | | | | | | | |

### Example 16

### Reversal of Diazepam-Induced Sedative-Hypnotic Effects in Rats Following Sublingual Administration of Flumazenil Sublingual Formulations

A preclinical study was performed in a rat sleep model in order to assess the therapeutic effects of flumazenil sublingual formulations in reversing diazepam-induced sedative effects following administration of the flumazenil formulations to Sprague-Dawley™ (SD) rats.

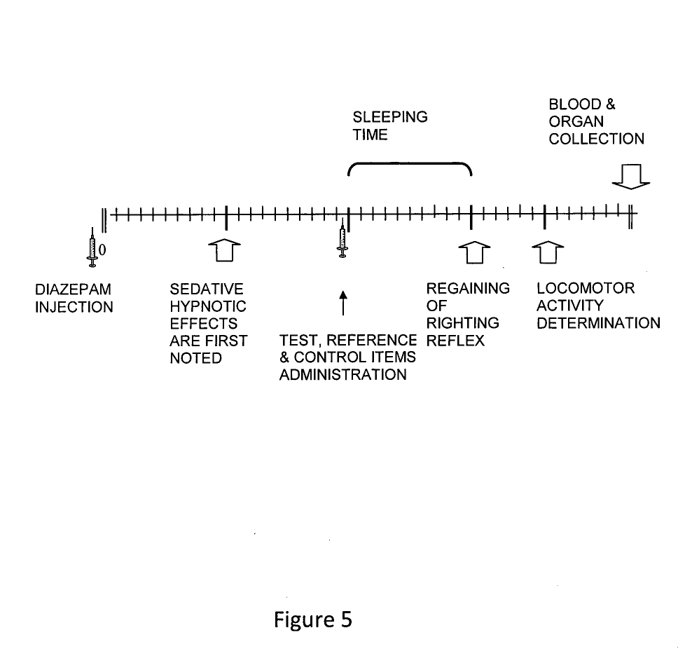

This study included two phase: the preliminary phase and the main phase. The aim of the preliminary phase was to determine the appropriate dose levels of the Induction (diazepam) and Reference (flumazenil) Items, both of which were administered i.v. The main phase included administration of the Test Items (flumazenil formulations) by the sublingual (SL) route after induction of sedative-hypnotic effects. The study protocol is summarized in FIG. 5.

In the preliminary and main phases final volumes of 2.5 ml or 5.05 ml of flumazenil USP solution at concentration of 2 mg/ml were used, respectively. The 2.5 ml (and 5.05 ml) solutions were prepared as follows:
1. 5.0 mg (10.1 mg) of flumazenil USP were weighed in a glass vial.
2. 125 ul (250 ul) of Tween 80 were added into the vial and the vial was mixed by hand until the flumazenil USP powder was moist.
3. 2.375 ml (4.8 ml) of 0.9% Sodium Chloride Injection were added to the vial.
4. The vial was gently mixed by hand until no sediment was observed.
   FLUMUP Placebo (Control Item) was prepared from the non active excipients.

Animals and Sleep Induction:

Rat/Hsd: Sprague-Dawley™ (Harlan Laboratories Israel) in good health, 7-8 weeks of age at study initiation, with weight variation that did not exceed ±20% were used as follows: Preliminary Phase (7 days)—n=8 (4 [symbol] & 4 [symbol] ); Main Phase (6 days)—n=32 (1 [symbol] & 16 [symbol] ). Diazepam (Induction Item) was injected intravenously as supplied by the manufacturer. During the acclimation and throughout the entire study duration, animals were housed within a limited access rodent facility and kept in groups of maximum 2 animals/cage, were fed ad libitum and were allowed free access to drinking water.

Preliminary Phase:

The experimental groups are listed in Table 27 below. This phase consisted of two groups of n=4 male and female SD rats/group. The first group was subjected to induction of sedative-hypnotic effects by IV injection of diazepam and served as an untreated control group, while the second group was injected in addition with the Reference Item flumazenil USP by the IV route.

Dose Level of Diazepam for Sleep Induction:
1. Untreated Control Group—Males:
a. Animal no. 14 was injected with a dose level of 10 mg/kg and ‘Sleep’ was induced for 46 seconds.
b. Animal no. 2 was injected with a dose level of 15 mg/kg and ‘Sleep’ was induced for 130 seconds. After a supplement dose level of 5 mg/kg ‘Sleep’ was induced for additional 850 seconds.
c. Animal no. 5 was injected with a dose level of 20 mg/kg and ‘Sleep’ was induced for 1700 seconds.
2. Reference Item Group—Males:
a. Animal no. 3 was injected with a dose level of 20 mg/kg and ‘Sleep’ was induced for 328 seconds until administration of the Reference Item flumazenil USP.
b. Animal no. 4 was injected with a dose level of 20 mg/kg and died immediately following injection.
3. Untreated Control Group—Females:
a. Animal no. 11 was injected with a dose level of 20 mg/kg and ‘Sleep’ was induced for 240 seconds. After a supplement dose level of 5 mg/kg ‘Sleep’ was induced for additional 1180 seconds.
b. Animal no. 15 was injected with a dose level of 25 mg/kg and died immediately following injection.
4. Reference Item Group—Females:
a. Animal no. 13 was injected with a dose level of 20 mg/kg and ‘Sleep’ was induced for 180 seconds. After a supplement dose level of 5 mg/kg ‘Sleep’ was induced for additional 300 seconds, until administration of the Reference Item flumazenil USP.
b. Animal no. 14 was injected with a dose level of 20 mg/kg and a supplement dose level of 5 mg/kg 125 seconds later, during its arousal. ‘Sleep’ was then induced for additional 322 seconds until administration of the Reference Item flumazenil USP.

Dose Level of Flumazenil:
The dose level of flumazenil USP was 2 mg/kg, excluding animal No. 14 which was injected with a second dose level of 2 mg/kg 5 minutes after the first dose. Flumazenil USP was injected at a volume dosage of 1 ml/kg.

TABLE 27
Preliminary phase experiment.

| | | Induction | | | | | Treatment | | | | |
|---|---|---|---|---|---|---|---|---|---|---|---|
| | | (Diazepam IV injection) | | | | | (Flumazenil USP IV injection) | | | | |
| | | | | Second Dose | | | | | Second Dose | | |
| | | | | | | Time | | | | | Time |
| | | First Dose | | | | Post | First Dose | | | | Post |
| | | Dose | Volume | Dose | Volume | First | Dose | Volume | Dose | Volume | First |
| Group | Animal | Level | Dosage | Level | Dosage | Dose | Level | Dosage | Level | Dosage | Dose |
| No. | No. | (mg/kg) | (ml/kg) | (mg/kg) | (ml/kg) | (sec.)* | (mg/kg) | (ml/kg) | (mg/kg) | (ml/kg) | (sec.)* |
| 1M | 2 | 15 | 3 | 5 | 1 | 182 | Untreated control | | | | |
| | 5¹ | 20 | 4 | 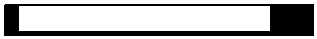 | | | | | | | |
| 1F | 11 | 20 | 4 | 5 | 1 | 311 | | | | | |
| | 15 | 25 | 5 | FDC | | | | | | | |
| 2M | 3 | 20 | 4 | 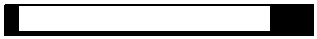 | | | 2 | 1 | 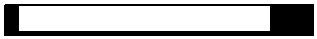 | | |
| | 4 | 20 | | FDC | | | Not applicable | | | | |
| 2F | 13 | 20 | 4 | 5 | 1 | 232 | 2 | 1 | | | |
| | 14 | 20 | | | | 125 | 2 | 1 | 2² | 1 | 300 |

¹Animal No. 1 was injected with diazepam at an initial dose level of 10 mg/kg, followed by a paravenous injection of the second dose, thus was removed from the study and replaced with animal No 5.
²Animal No. 14 was subjected to a second dose of flumazenil USP
FDC = Found Dead in the Cage;
IV = Intravenous;
Shaded area - A second dose of diazepam or flumazenil USP was not required
*The Time was recorded from the end of the previous administration of either diazepam or flumazenil USP.

Main Phase:

The experimental groups are listed in Table 28. The main phase consisted of 4 groups of n=8 male and female SD rats/group. Induction of sedative-hypnotic effects, as measured by Sleeping Time and locomotor activity, was carried out by two intravenous injections of diazepam at an interval of approximately 2 minutes. The Test, Reference and Control Items were administered twice at interval of 2 minutes as well, about 5 minutes after the second dose of diazepam. Both Test Items FLUMUP-F22 (F22, Table 26 below) and FLUMUP-F26 (F26, Table 26 below) were administered by the sublingual route at a constant dose volume of 0.1 ml/animal per administration. An additional group was administered with FLUMUP Placebo under identical experimental conditions and served as a Control group. A fourth group was subjected to IV injection of the Reference Item under identical experimental conditions. Animals were placed in the activity cage immediately following arousal from ‘Sleep’ or 10 minutes post the second dose of the respective treatment if arousal was not evident.

Induction of Sedative-Hypnotic Effects:

Males were injected with an initial dose level of 15 mg/kg and approximately 2 minutes later an addition of 5 mg/kg was administered. Females were injected with an initial dose level of 20 mg/kg and approximately 2 minutes later an addition of 5 mg/kg.

Treatment:

(i) The Test and Control Items were administered by the sublingual route twice at an interval of approximately 2 minutes about 5 minutes post the second injection of diazepam. (ii) The Reference Item flumazenil USP was injected twice intravenously at an interval of approximately 2 minutes about 5 minutes post the second injection of diazepam. The Test, Reference and Control Items were administered at a constant dose volume of 0.1 ml/animal per administration over approximately 15 seconds.

TABLE 28
Main phase experiment

| | | Induction (Diazepam IV injection) | | | | | Treatment | | | | |
|---|---|---|---|---|---|---|---|---|---|---|---|
| | | First Dose | | Second Dose | | | First Dose | | | Second Dose | |
| | | Dose | Dose | Dose | Dose | Time | Test | Dose | Time | Dose | Time |
| Group | Animal | Level | Level | Level | Level | Post First | Material & | Volume | Post First | Volume | Post First |
| No. | No. | (mg/kg) | (ml/kg) | (mg/kg) | (ml/kg) | Dose (sec.)* | Route | (ml/An) | Dose (sec.)* | (ml/An) | Dose (sec.)* |
| 3M | 21 | 15 | 3 | 5 | 1 | 124 | FLUMUP | 0.1 | 315 | 0.1 | 130 |
| | 22 | | | | | 125 | Placebo | | 315 | | 145 |
| | 23 | | | | | 131 | SL | | 319 | | 131 |
| | 62 | | | | | 124 | | | 329 | | 87³ |
| 3F | 41 | 20 | 4 | | | 129 | | | 320 | | 134 |
| | 42 | | | | | 126 | | | 317 | | 151 |
| | 43 | | | | | 125 | | | 320 | | 138 |
| | 44 | | | | | 123 | | | 308 | | 127 |
| 4M | 65 | 15 | 3 | 5 | 1 | 133 | FLUMUP | 0.1 | 316 | 0.1 | 139 |
| | 26 | | | | | 120 | F22 | | 297 | | 132 |
| | 27 | | | | | 125 | SL | | 316 | | 143 |
| | 28 | | | | | 116 | | | 312 | | 142 |
| 4F | 73 | 20 | 4 | | | 127 | | | 322 | | 147 |
| | 46 | | | | | 125 | | | 322 | | 150 |
| | 47 | | | | | 125 | | | 311 | | 126 |
| | 48 | | | | | 128 | | | 319 | | 136 |
| 5M | 29 | 15 | 3 | 5 | 1 | 124 | FLUMUP | 0.1 | 318 | 0.1 | 135 |
| | 30 | | | | | 116 | F26 | | 321 | | 138 |
| | 61 | | | | | 128 | SL | | 317 | | 142 |
| | 32 | | | | | 123 | | | 329 | | 132 |
| 5F | 49 | 20 | 4 | | | 125 | | | 319 | | 143 |
| | 50 | | | | | 129 | | | 311 | | 138 |
| | 51 | | | | | 158 | | | 316 | | 147 |
| | 52 | | | | | 125 | | | 319 | | 138 |
| 6M | 33 | 15 | 3 | 5 | 1 | 130 | Flumazenil | 0.1 | 319 | 0.1 | 135 |
| | 34 | | | | | 130 | USP | | 315 | | 135 |
| | 35 | | | | | 135 | IV | | 315 | | 135 |
| | 36 | | | | | 130 | | | 315 | | 135 |
| 6F | 53 | 20 | 4 | | | 130 | | | 315 | | 135 |
| | 54 | | | | | 130 | | | 315 | | 135 |
| | 55 | | | | | 130 | | | 315 | | 135 |
| | 56 | | | | | 130 | | | 315 | | 135 |

SL = Sublingual; IV = Intravenous;
*The Time was recorded from the end of the previous administration of either diazepam or the treatment.

Observations:

Sleeping Time Determination:

Sleeping time was determined for all animals. The clock time post the second diazepam injection was recorded when the test animal was no longer capable to maintain its Righting Reflex, which defined as the animal's failure of righting 2 times within 30 seconds. Thereafter the test animal was placed on its back in a bedded standard cage. The clock time was recorded again after the administration of the Test, Reference and Control Items and when the animal regained its Righting Reflex.

In the preliminary phase Sleeping Time corresponded to the time elapsed between last injection of diazepam and animals' regaining of its Righting Reflex. In the main phase, Sleeping Time corresponded to the time elapsed between the second administration of the Test, Reference or Control Items and animal's regaining of its Righting Reflex.

Locomotor Activity Determinations:

Locomotor activity was measured on 2 occasions, during acclimation period for baseline control determination and at a fixed time point that was determined to be as soon as possible following animals' regaining its Righting Reflex or 10 minutes following the second administration of the Test, Reference and Control Items in case the animal still lack of its Righting Reflex.

Each rat was placed in the center of a special designed commercial activity cage, measuring 40×40×46 cm (TruScan Photo Beam Activity System-Coulbourn Instruments, Allentown, Pa., USA) for a duration of 15 minutes. With the use of software provided with the system, the following parameters were recorded for each 5-min interval during the 15 min testing session: (1) Total distance covered by the animal; (2) Number of rears exhibited by the animal, i.e. vertical body extension while standing on its hind feet; and (3) Time spent in the activity cage's center area for the entire session.

Blood and Organ Collection:

A blood sample was collected from all animals assigned to the main phase following completion of the locomotor activity determination by retroorbital sinus bleeding under light CO₂ anesthesia. Following blood (at least 0.5 ml/animal) collection animals were euthanized by CO₂ asphyxiation and the organs were collected from main phase animals (brain, liver, left kidney, heart, lung, spleen, tongue with the lower jaw, esophagus and stomach).

Following centrifugation of the whole blood samples, at least 150 ul of plasma were collected into pre-labeled (by HBI Study No.; Group No.; Animal No. and time post administration) cryo plastic screw tubes, frozen in liquid nitrogen and then kept on dry ice until transference to storage at (−70)-(−80)° C.

Data Evaluation:

Evaluation was based on the relative recorded changes in the measured Sleeping Time and locomotor activity, obtained from the Test & Reference Items-treated animals vs. those of the Control Item group. Data analysis of all measurable parameters was performed by the use of the following methods in order to determine significance of treatment effects: Software: GraphPad Instat®—Version 3.02 (Statistical Method: 1-Way ANOVA; Dunnett Multiple Comparisons Test for locomotor activity parameters; Kruskal-Wallis Test for Sleeping Time data) Microsoft” Excel 2000.

Results:

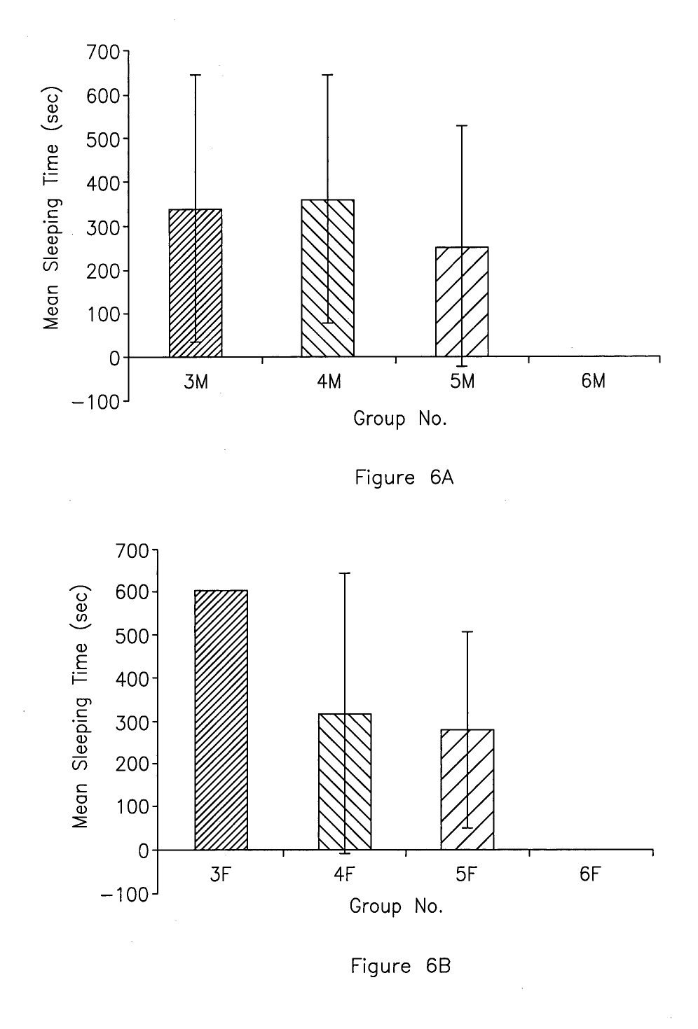

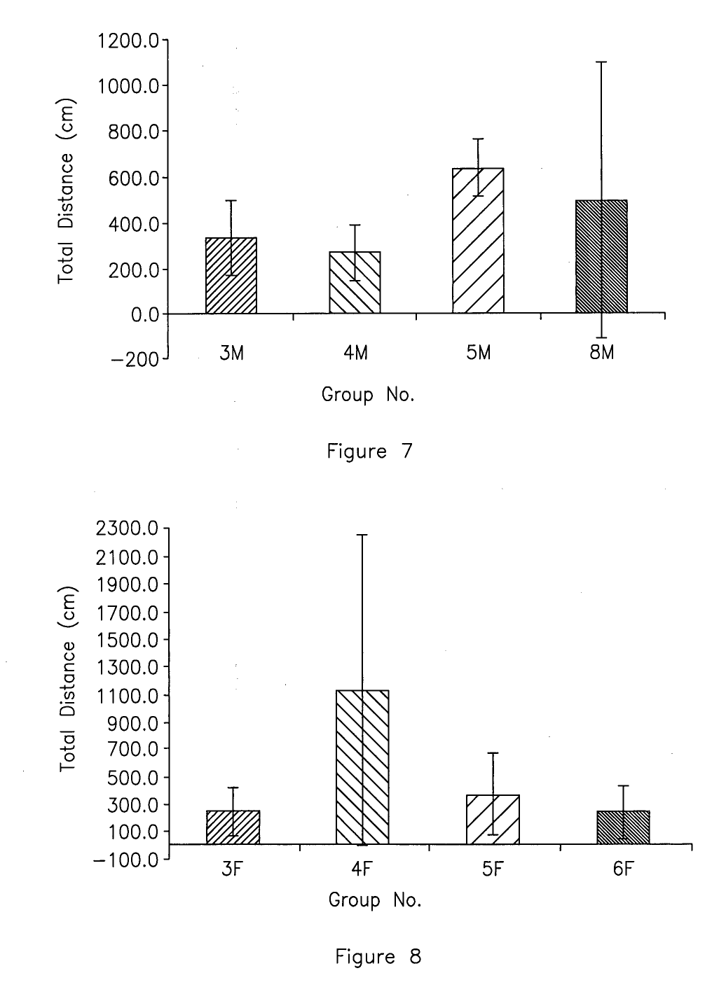

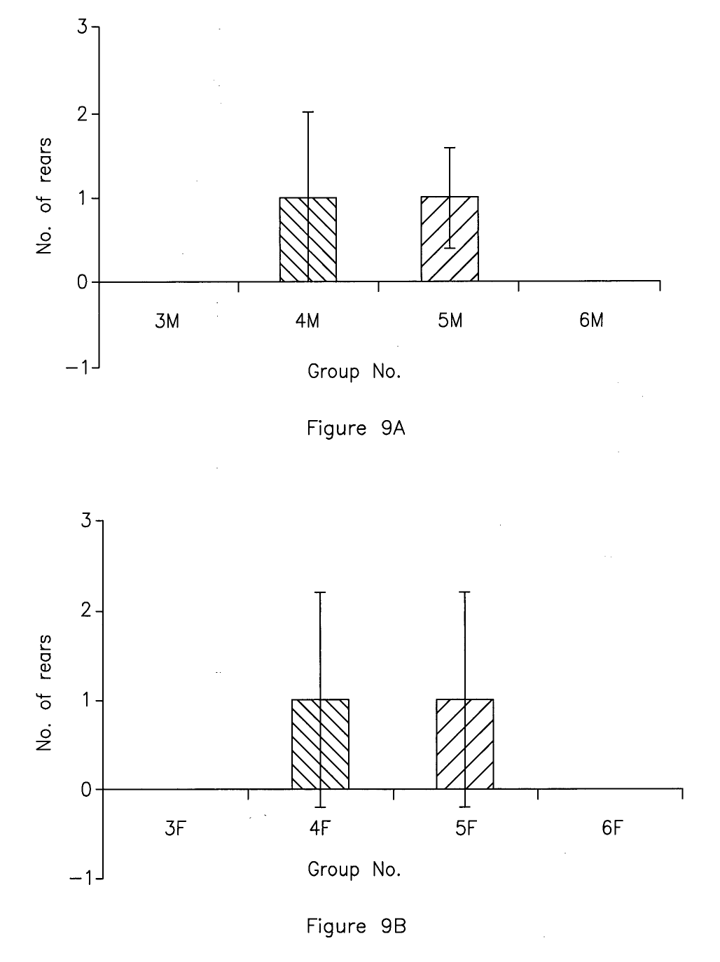

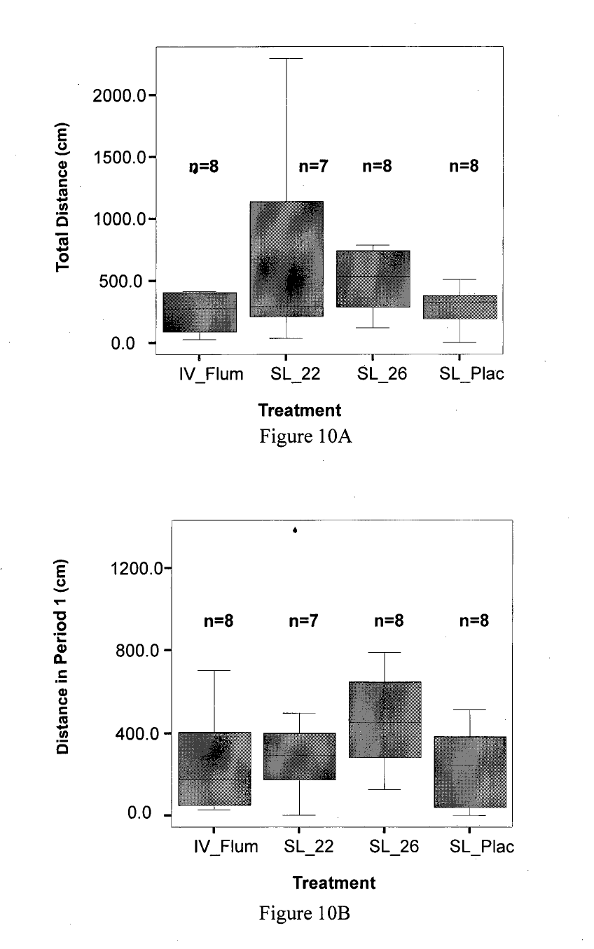

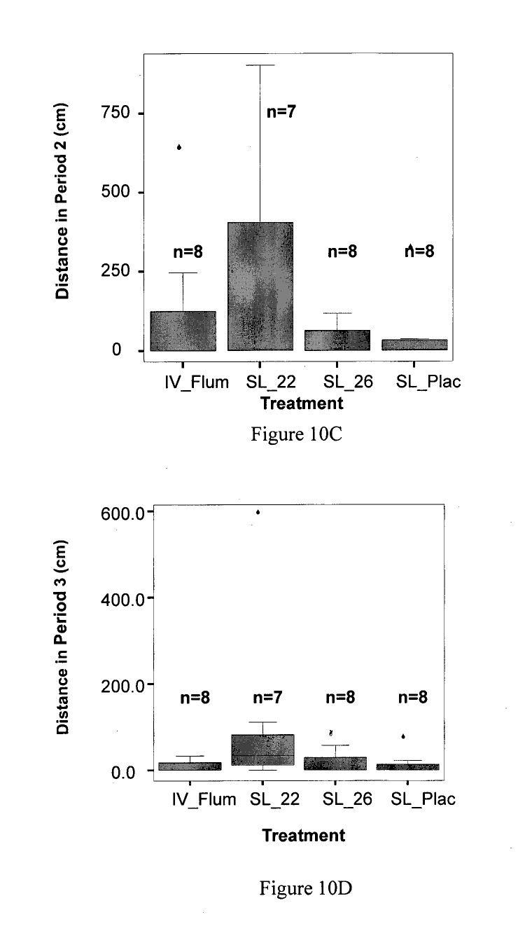

Sleeping Time(s) of the Main Phase are shown in FIGS. 6A-B, Locomotor Activity, specifically, the total distance values and number of rears recorded are shown in FIGS. 7-8. The numbers of rears for all groups is presented in FIGS. 9A-B. FIG. 10 presents the distance of movement at the first (A), second (B) and third time periods (C) and the total distance of movement (D) in the cages of all animals upon treatment with Flumzenil formulation administered sublingually, with respect to flumazenil administered i.v. and control (placebo, sublingual).

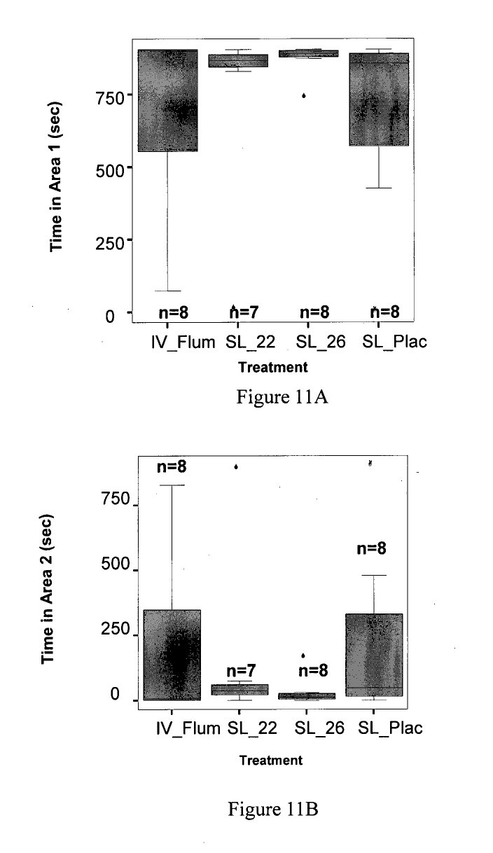

As shown in the aforementioned figures, Locomotor Activity, specifically, the total distance values and number of rears recorded in males and females of all groups following treatment, were lower in comparison to the baseline measurements. The results further indicate that the flumazenil formulations of the invention induced movement to longer distances as compared to IV Flumzenil and s/1 placebo (e.g. FIG. 10A). It is also shown that the countering effect induced by sublingual administration of the Flumzenil formulations of the invention is maintained throughout the experiment time (periods 1-3, FIGS. 10B-C) and in fact it is maintained even after 60 min. (period 3; e.g. FIG. 10D). Although not statistically significant, mean group values of total distance recorded in males treated with FLUMUP-F26 (5M) was relatively higher comparing to that of the other groups and in the Reference Item treated group only one out of the four males exhibited higher values of total distance in comparison to controls. FLUMUP-F22 treated males (4M) exhibited mean group values of total distance similar to that of the Control group (3M). Mean group time spent in the extent of the activity cage of the males treated with the FLUMUP-F26 or the Reference Item was relatively higher comparing to that of the controls. Mean group number of rears was similar in the males of all groups. Another surprising observation is that sublingual administration of the Flumzenil formulations of the invention induced movement in the peripheries of the cages (Area 1; FIG. 11A), which is a normal behavior. However, rats stayed in the central area of the cage (Area 2; FIG. 11B), where they do not normally spend time, when treated with placebo or with Flumzenil USP administered i.v.

Two out of four females treated with FLUMUP-F22 (4F) exhibited remarkably higher values of total distance comparing to that of the Control group, while total distance covered by the remaining two females in the group was similar to that of controls. No statistical significant difference was noted in mean group values of total distance covered by the females of FLUMUP-F26 and Reference Item treated groups in comparison to controls. Mean time spent in the extent of the activity cage of FLUMUP-F26 treated females was relatively higher comparing to that of the Control group. Mean group number of rears was similar in the females of all groups.

In consideration of the selected sublingual route of administration which requires general anesthesia in order to avoid swallowing of the administered substance, diazepam was injected intravenously at extremely high dose levels to induce hypnotic effects. In males a total dose of 20 mg/kg (i.e. an initial dose level of 15 mg/kg and an additional dose of 5 mg/kg 2 minutes later) and in females a total dose of 25 mg/kg (i.e. an initial dose level was mg/kg and an additional dose of 5 mg/kg 2 minutes later). It should be noted that the recommended dose level of diazepam for induction of sedation in rats by intravenous injection is 2 mg/kg. Furthermore, diazepam injections at a single dose level of 20 mg/kg to males and 25 mg/kg to females resulted in animals' death and lower dose level did not induce hypnotic effects.

Flumazenil, which is used for reversal of benzodiazepine effect by intravenous route, is injected at an initial dose of 0.2 mg over a period of 15 seconds to adult patients. Addition of 0.1 mg of flumazenil may be repeated at intervals of 60 seconds until the desired degree of consciousness is obtained and up to a total dose of 1 mg. Repeated injections may be used at intervals of 60 seconds up to a total dose of 2 mg. In this study, the Test and Reference Items were administered at a total dose of 0.8 mg/animal (2 administrations of 0.4 mg/animal at an interval of 2 minutes), which is extremely higher than the human dose. However, it was essential to counteracting the diazepam severe hypnotic effects which were induced in this model.

In continuation to the above-mentioned dose levels, it should be noted that the Reference Item treated animals regained their righting reflex very shortly following injection; however their locomotor activity was still very low and similar to that of the control animals.

The results of this study indicate a therapeutic potential in terms of reversing the diazepam-induced sedative-hypnotic effects of FLUMUP-F22 & FLUMUP-F26 (Batch No's: F22 & F26) administered twice at 2 minutes interval by the sublingual route at a dose volume of 0.1 ml (corresponding to 0.4 mg) per administration to male and female Sprague-Dawley rats.

### Example 17

### Safety and Efficacy of a Sublingual Formulation of Flumazenil to Reverse the Effect of Hypnotic Drugs (Zolpidem or Brotizolam) in Humans

The purpose of this study was to evaluate the safety and effectiveness of treatment with sublingual (s/l) dosage form of flumazenil in healthy volunteers (n=20) for reversing the hypnotic effect of sleeping pills. The present clinical study presents short-term safety and tolerability data, the psychomotor/cognitive and behavioral effects of flumazenil and the degree and the duration of action in a single use of flumazenil administered in doses of 0.4 mg per 100 μl up to 1.6 mg in a sublingual spray using a formulation comprising the following non-active excipients: Ethanol, Propylene glycol, 30% HPCD in citric buffer 10 mM pH 4.0, Menthol in EtOH (1:1 w/w) and propylparaben/methylparaben.

Study Design:

All subjects (n=20; 10 males, 10 females) were tested for the safety and efficacy of s/l administration of flumazenil to reverse the sleep/hypnotic effect of zolpidem or brotizolam.

Subjects arrived at the clinic after having partial sleep deprivation. Sleep induction was achieved with brotizolam for 10 subjects (Study Arm A) and with zolpidem for 10 subjects (Study Arm B). After taking the hypnotic drug subjects were allowed to sleep for 1.5 hours. They were then awakened and a series of functional tests and questionnaires were performed (visual analog scale rating for sleepiness, ability to focus, effectiveness in work; immediate word recall test—iWRT, Digit Symbol Substitution test—DSST; and Profile Of Mood Status—POMS). Then, subjects were awakened and randomly treated by sublingual flumazenil, 0.4 mg (n=10) and 1.6 mg (n=10) or placebo and were re-evaluated for sleepiness and psychomotor performances (same tests) 20 min and 1 hour following administration. In the next visit (7±2 days apart), the experiment was repeated with placebo or flumazenil.

Evaluation of response and safety data for each study arm was performed for the first 10 randomized subjects (5 subjects with brotizolam and 5 subjects with zolpidem). Since differences between placebo and flumazenil at that stage (still blinded) did not seem significant, a decision to double the hypnotic drug doses was taken, as well as increasing the flumazenil dose to 1.6 mg thus enabling dose ranging examination in this trial by analyzing the data.

### Randomization and Blinding

Each subject was treated by both flumazenil and placebo in either one of the treatment visits. Subjects were randomized for “the order of treatment”. Flumazenil and Placebo were packaged identically. Labels were clearly indicated the randomization number the treatment assigned for each treatment day: Treatment for the 1st day was labeled “A” and for the 2nd day “B”.

### Inclusion Criteria

Subjects met all of the following inclusion criteria at the screening stage, in order to be eligible for enrolment into the study:

1. The subject signed an informed consent.
2. Male or female aged ≧18.
3. Body mass index ≧18.5 and <32 kg/m²
4. Normal sleeping habits.
5. Good health.
6. Negative for any use of illicit drug, alcohol (ethanol) and stimulants.

### Exclusion Criteria

1. Any use of medications within 1 month prior to screening visit, except for contraceptive pills.
2. Any sleep associated complains.
3. Previous exposure to benzodiazepines and/or non-benzodiazepine hypnotic drugs within 3 months prior to study initiation.
4. History of Epilepsy and or anti-epileptic drugs.
5. Excessive caffeine consumption (2:500 mg per day).
6. Pregnancy or breast feeding.
7. Night shift workers within 1 month prior to the screening visit.
8. Clinically relevant ECG abnormalities.
9. History of alcohol or drug abuse within 3 years prior to the screening visit.
10. Cognitive Behavioral Therapy (CBT) started within 1 month prior to the screening visit.

11. Known hypersensitivity to drugs of the same class as the study treatment, or any excipients of the drug formulation.
12. Treatment with another investigational drug within 1 month prior to the screening visit.
13. History of severe head injury.

### Study Visits and Overall Duration

The following visits were included in the study:

Visit 1—Screening visit: No screen's failures were reported.

Visit 2&3—Treatment Visits, which included the following procedures:
Prior to administration of medication—Full physical examination, including the sublingual and the oral area; Vital signs measurements; Adverse events and Concomitant medication inquiry.
Sleep induction: Sleep/hypnotic drug:
   Arm A: Brotizolam (benzodiazepine)—10 subjects (5-0.25 mg and 5-0.5 mg)
   Arm B: Zolpidem (non-benzodiazepine)—10 subjects (5-10 mg and 5-20 mg).
All subjects were allowed to sleep for 90 min, then all subjects were awakened to perform a battery of cognitive/behavioral tests (Hypnotic Baseline). After completing the battery of tests, at time set as time 0, s/l flumazenil or placebo (randomly and blindly) were administrated. 20 minutes post administration of flumazenil or Placebo, subjects were asked to perform the performance tasks (see below) and vital signs were measured. Subjects had to perform the same tasks 60 minutes post administration of flumazenil or Placebo.
The following safety measurements were assessed after performing the last performance tasks: Full physical examination, including the sublingual and the oral area; Neurological assessment, Vital signs measurements; Adverse events (AE) inquiry.
The following performance tasks were assessed: Visual Analogue Scale (VAS) for Sleepiness, ability to focus, and effectiveness in performance; Scores of the Profile Of Mood States (POMS) Brief form for sleepiness and vigilance; Total score on the digit symbol substitution test (DSST) and Total score on the immediate Word Recall Test (iWRT).

The scores of each of the above tasks under flumazenil were compared to placebo. In addition, scores with both treatments (flumazenil and placebo) at baseline were compared to scores at 90 min. after sleep/hypnotic drug administration (Hypnotic Baseline).

Response to treatment (R) was defined as follows: Evaluation of efficacy variables for flumazenil treatment versus efficacy variables for placebo treatment, where Good Response to treatment (GR) corresponds to R≧30% and Very Good Response to treatment (VGR) corresponds to R≧50%.

Within a 7±2 days from study visit 2, visit 3 was conducted according to the visit plan described for visit 2. Subjects that were randomized for treatment with flumazenil in the first treatment day were treated with placebo in the second treatment and vice versa. Subjects participated in the study for up to 5 weeks.

### Statistical Methodology

Each subject was his own control. Mean and standard deviation in the above scores were analyzed using the following statistics tests: student-t test and ANOVA. Excel and Access softwares were used for data collection. SPSS software was used for data analysis. Statistical significance was defined as p<0.05.

Drug preparation (FLUMUP)

FLUMUP formulation (Formulation D, Table 11 above) was filled into glass bottles (5 ml) and screwed with pumps (Pump 100 μl Pfeiffer; dip tube length 40.5 mm) fitted with an Actuator delivering 0.1 ml (metered dose) per puff. Pumps were routinely tested for accuracy and reproducibility by the manufacturer. Filled bottles were stored at room temperature. Each puff produced 0.1 ml, equal to 0.4 mg of the FLUMUP (flumazenil concentration of 4 mg/mL). The study was designed for 0.4 mg and/or 1.6 mg (1 or 4 puffs). It is noted that the recommended dose for i.v. administration is up to 3 mg/hr.

### Preparation of FLUMUP:

Citric buffer 10 mM pH 4.0 was prepared from citric acid anhydrous 0.1199% w/v and sodium Citrate dihydrate 0.1105% w/v dissolved in water and pH was adjusted to 4.0±0.2 (if needed). HPCD 30% w/w in Citric buffer 10 mM pH 4.0 solution was prepared. Menthol in EtOH solution was prepared by dissolving Menthol in Ethanol absolute in a ratio of 1/1 w/w. Propylparaben and Methylparaben were dissolved in Ethanol in ratio 0.02/0.18/10 w/w/w.

The required amount of flumazenil was weighed in a glass vessel following addition of EtOH, parabens solution, Menthol solution; PG and HPCD 30% in Citric buffer 10 mM pH 4.0 solution. The mixture was stirred using a rotor stirrer until a clear solution was obtained. Placebo (Formulation D′; Table 13 above) was prepared similarly.

It is noted that due to administration of low volume of flumazenil (0.1-0.4 ml per day) the amounts of each excipient in the active formulation and in the placebo are far below the maximal FDA approved daily dosages for the i.v. formulation (Romazicon®).

### Results:

As stated, the study was performed on 20 participants, 10 males and 10 females. Of the subjects, 10 subjects received brotizolam and 10 subjects received zolpidem for sleep induction. All participants have completed the study. No drop-outs or any side effects were reported. All physical examinations, neurological assessment and local mouth and buccal examination, cardiac and lung examination, and ECG evaluation were not adversely affected by flumazenil with respect to placebo. Vital signs (pulse, respiration), temperature and blood pressure were within normal values for flumazenil and placebo. None of the participants complained of any unusual sensation, pain, nausea, abdominal pain or any other potential side effect, with both doses of flumazenil. Table 15 presents the general characteristics of the participants:

TABLE 15
Participants characteristics

| n = 20, 10men + 10 women | Average ± SEM | range |
|---|---|---|
| Age | 28.8 ± 5.7 | 19-42 |
| BMI | 23.6 ± 3.5 | 18.7-31.8 |
| Baseline pulse rate | 74.4 ± 9.3 | 64-98 |
| Baseline systolic BP | 118.6 ± 11.6 | 100-136 |
| Baseline diastolic BP | 73.4 ± 7.0 | 64-89 |

As stated, since in the first 10 participants (5 with brotizolam and 5 with zolpidem for sleep induction) there were no significant differences between A or B oral spray (blinded at that time) the doses were increased in both arms and in the hypnotic drug while maintaining blinding and in the following 10 participants the dose of flumazenil was 1.6 mg. This led to significant differences between flumazenil and Placebo in both arms.

Table 16 describes the baseline performance of the participants, and the effects of sleep induction, in both arms.

TABLE 16
Baseline performances and the effect of sleep induction

| | Baseline | Baseline sleep (after hypnotic, before oral spray) | | |
|---|---|---|---|---|
| | (visit 1) | Brotizolam | Zolpidem | All - BL Sleep |
| Pulse rate | 74.4 ± 9.3 | 69.8 ± 1.8 | 77.5 ± 3.5 | 73.7 ± 2.2 |
| Systolic BP | 118.6 ± 11.6 | 116.9 ± 4.8 | 119.5 ± 4.0 | 118.2 ± 2.8 |
| Diastolic BP | 73.4 ± 7.0 | 69.0 ± 2.8 | 71.4 ± 1.9 | 70.2 ± 1.6 |
| iWRT | 12.1 ± 0.7 | 7.6 ± 1.3 | 4.1 ± 0.3 | 5.9 ± 0.8 |
| Vigilance | 7.8 ± 0.4 | 2.3 ± 0.4 | 1.4 ± 0.3 | 1.8 ± 0.2 |
| Focus ability | 7.8 ± 0.3 | 2.4 ± 0.4 | 1.5 ± 0.4 | 2.0 ± 0.3 |
| Effectiveness | 8.3 ± 0.2 | 2.3 ± 0.3 | 1.5 ± 0.4 | 1.9 ± 0.2 |
| POMS- fatigue | 3.2 ± 0.7 | 7.2 ± 1.2 | 9.3 ± 1.4 | 8.3 ± 0.9 |
| POMS-Alertness | 12.0 ± 0.9 | 4.3 ± 1.0 | 1.9 ± 1.0 | 3.1 ± 0.8 |
| DSST | 88.5 ± 3.5 | 76.3 ± 7.7 | 62.5 ± 3.6 | 69.4 ± 4.5 |

Interim abbreviated report summarizing safety and evaluation of response to treatment was prepared for the first 10 subjects enrolled to the study. Since no significant differences were observed between substance A and B (flumazenil/placebo), the dosage had been increased:

TABLE 17
Interim (planned) analysis (N = 10)

| | Δ parameter 20 min after spray | | | Δ parameter 60 min after spray | | |
|---|---|---|---|---|---|---|
| | A | B | P | A | B | P |
| iWRT | 2.8 ± 1.2 | 2.9 ± 1.4 | .47 | 4.7 ± 1.9 | 3.7 ± 2.1 | .29 |
| Vigilance | 2.3 ± 0.7 | 1.5 ± 1.1 | .26 | 3.7 ± 0.9 | 3.9 ± 1.2 | .44 |
| Focus ability | 1.7 ± 0.7 | 1.1 ± 0.8 | .27 | 2.9 ± 0.7 | 3.3 ± 1.2 | .38 |
| Effectiveness | 1.9 ± 0.8 | 1.8 ± 0.8 | .47 | 3.5 ± 0.8 | 4.0 ± 1.0 | .33 |
| POMS- fatigue | −1.5 ± 0.9 | −1.9 ± 1.3 | .39 | −2.7 ± 1.0 | −4.9 ± 1.0 | .05 |
| POMS-Alertness | 2.7 ± 1.1 | 0.8 ± 1.4 | .11 | 3.3 ± 1.5 | 3.5 ± 1.5 | .45 |
| DSST | 12.1 ± 1.9 | 13.7 ± 3.6 | .35 | 14.3 ± 2.9 | 17.7 ± 4.2 | .27 |

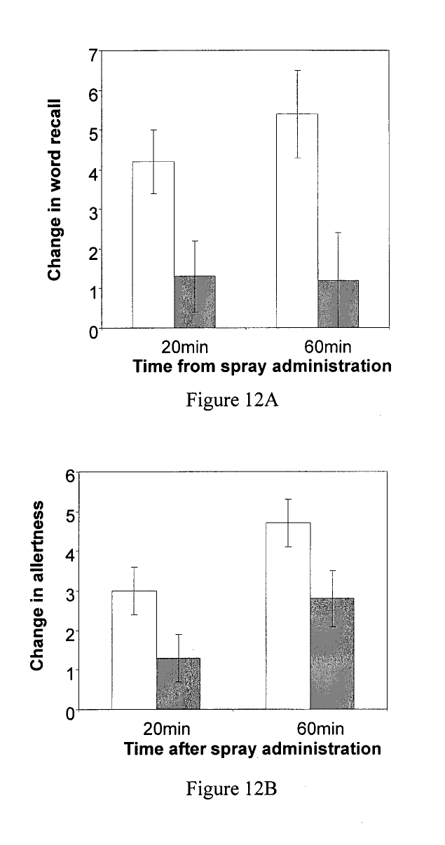

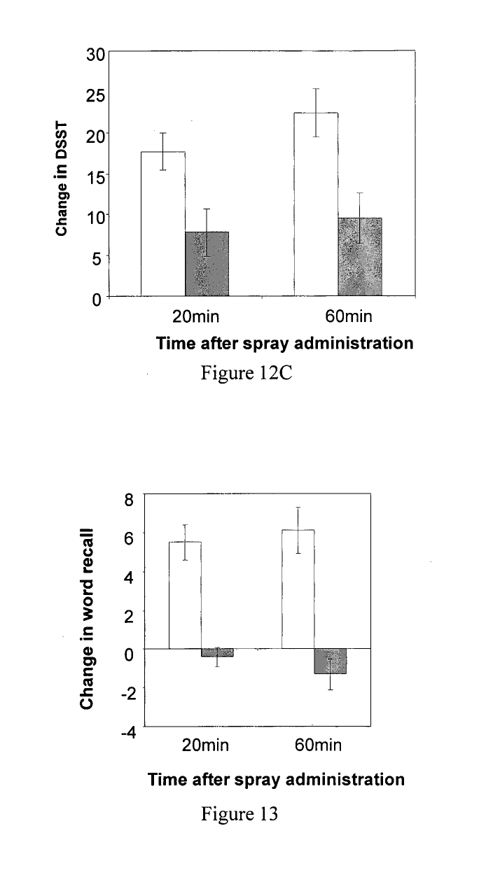

Following sleep and baseline sleep evaluation, flumazenil (FIGS. 12A-C; white columns) or placebo (FIGS. 12A-C; gray columns) were given under the tongue, and efficacy EP's re-assessment took place 20 min and 60 min thereafter. The following table (Table 18) and figures (FIGS. 12A-C) present the differences between flumazenil (white columns) and placebo (gray columns). Since baseline values following sleep were different in visit 2 and 3, the results are expressed as the change with flumazenil or placebo from baseline sleep results per the specific visit when they were given. As stated above, for the first 10 subjects there were no significant differences between flumazenil and placebo. For this reason for the last 10 participants the dose of the hypnotic was doubled, and the dose of flumazenil/placebo was escalated by 4 (to 1.6 mg). Indeed, for the last 10 participants the response is rated as very good (over 30% differences between flumazenil and placebo).

TABLE 18
Performance and subjective - FLUMUP vs. Placebo (N = 20)

| | Δ parameter 20 min aft spray | | | Δ parameter 60 min aft spray | | |
|---|---|---|---|---|---|---|
| | Flumazenil | Placebo | P | Flumazenil | Placebo | P |
| Pulse | −2.9 ± 1.3 | −4.8 ± 1.8 | .22 | −3.4 ± 1.6 | −3.6 ± 1.8 | .47 |
| Systolic BP | −5.5 ± 2.3 | −3.0 ± 2.2 | .22 | −6.1 ± 2.7 | −0.1 ± 2.2 | .03 |
| Diastolic BP | −0.5 ± 1.5 | −2.2 ± 1.8 | .17 | −2.3 ± 1.2 | 0.7 ± 1.9 | .09 |
| iWRT | 4.2 ± 0.8 | 1.3 ± 0.9 | .005 | 5.4 ± 1.1 | 1.2 ± 1.2 | .002 |
| Vigilance | 3.0 ± 0.6 | 1.3 ± 0.6 | .02 | 4.7 ± 0.6 | 2.8 ± 0.7 | .003 |
| Focus Ability | 2.7 ± 0.5 | 1.3 ± 0.5 | .03 | 4.3 ± 0.6 | 2.4 ± 0.7 | .003 |
| Effectiveness | 2.9 ± 0.6 | 1.5 ± 0.6 | .05 | 4.5 ± 0.6 | 2.5 ± 0.7 | .003 |
| POMS- fatigue | −3.7 ± 0.9 | −1.8 ± 0.8 | .09 | −5.5 ± 1.0 | −3.8 ± 0.8 | .14 |
| POMS-Alertness | 3.5 ± 0.9 | 0.6 ± 0.8 | .008 | 5.3 ± 1.2 | 1.9 ± 1.1 | .002 |
| DSST | 17.7 ± 2.3 | 7.8 ± 2.9 | .01 | 22.4 ± 3.0 | 9.5 ± 3.2 | .008 |

The following table (Table 19) and figure (FIG. 13) present the differences between flumazenil and placebo for the high dose study (FLUMUP 1.6 mg; 10 participants), as a function of word recall (iWRT):

TABLE 19
Performance and subjective - high dose FLUMUP subpopulation

| | Δ parameter 20 min aft spray | | | Δ parameter 60 min aft spray | | | % difference |
|---|---|---|---|---|---|---|---|
| | Flumazenil | Placebo | P | Flumazenil | Placebo | P | Flum vs. P |
| Pulse | −0.4 ± 1.9 | −5.1 ± 3.7 | .13 | −1.2 ± 2.7 | −3.8 ± 3.4 | .27 | 8 |
| Systolic BP | −4.7 ± 3.5 | −2.8 ± 3.6 | .38 | −1.7 ± 4.2 | −1.7 ± 3.7 | .5 | 59 |
| Diastolic BP | −2.0 ± 1.9 | −1.4 ± 2.2 | .41 | −2.6 ± 1.8 | 0.4 ± 3.6 | .22 | 70 |
| iWRT | 5.5 ± 0.9 | −0.4 ± 0.8 | .001 | 6.1 ± 1.2 | −1.3 ± 0.5 | .001 | 92 |
| Vigilance | 3.6 ± 0.8 | 1.1 ± 0.6 | .02 | 5.8 ± 0.8 | 1.6 ± 0.7 | .001 | 69 |
| Focus Ability | 3.7 ± 0.7 | 1.5 ± 0.7 | .02 | 5.8 ± 0.8 | 1.6 ± 0.9 | .001 | 59 |
| Effectivenes | 3.8 ± 0.7 | 1.1 ± 0.9 | .01 | 5.5 ± 0.7 | 1.0 ± 1.0 | .001 | 61 |
| POMS- | −5.8 ± 1.6 | −1.6 ± 1.0 | .04 | −8.2 ± 1.6 | −2.7 ± 1.4 | .01 | 72 |
| POMS- | 4.3 ± 1.5 | 0.3 ± 0.8 | .02 | 7.2 ± 2.0 | 0.3 ± 1.4 | .01 | 93 |
| DSST | 23.2 ± 3.4 | 1.8 ± 3.5 | .005 | 30.4 ± 3.6 | 1.3 ± 2.9 | .001 | 92 |

As can be seen, with flumazenil there was a significant improvement in words recall 20 min and 60 min following administration, compared to slight deterioration with placebo (probably due to continuation of hypnotic effect).

The results clearly show that treatments with brotizolam and zolpidem result in sleepiness and reduced neurocognitive functions, which is completely, and in a significant manner, reversed by sublingual administration FLUMUP but not by placebo. With low dose of hypnotics (Bondormin® 0.25 mg or 10 mg zolpidem) the initial effects on the performance functions were mild, and could not be better reversed by the low dose of flumazenil (0.4 mg). However, with the double hypnotic dose and 4 times FLUMUP dose the reversing action was robust, with FLUMUP superior over placebo by 59-93% in the various performance tasks. Thus, in terms of dosage it seems that the necessary effective FLUMUP dose is 1.6 mg.

In terms of time and duration of effect both 20 minutes and 60 minutes following the sublingual spray administration there were clear improvements in vigilance, alertness and performances with flumazenil compare to placebo. Thus, the duration of effect starts as soon as 20 min following administration and remains effective at least 60 min following administration. Surprisingly, the effect was even more robust after 60 minutes vs. the 20 minutes interval.

The results suggest that even the usage of a higher dose of drugs inducing sleep, which could result with a more effective treatment of insomnia during the night, would be reversed upon awakening by sublingual spraying of 1.6 mg FLUMUP.

### Example 18

### Passive Transdermal Delivery of Flumazenil with 3M Drug Delivery Drug-in-Adhesive Technology

The feasibility of transdermal delivery was evaluated for flumazenil and zolpidem. The target transdermal dose for flumazenil used in this assessment was 0.6 mg/hr. For zolpidem, the target transdermal dose was estimated from the oral dose (10 mg/day) and the oral bioavailability (70%).

The specific requirements for the dosing duration of flumazenil, are to provide a 6 hour delay of drug release after initial application of the patch, followed by a rise time within 30 minutes to a blood level of 20 ng/mL, followed by 1.5 hours of dosing to a target blood level of 40 ng*hr/mL. The patch is removed after the 2 hours of dosing. The patch is applied in the evening and removed in the morning. For zolpidem, the dosing requirements are to reach a blood level of 60 to 100 ng/mL within one hour of patch application, followed by 4 hours of constant delivery at that blood level, followed by decreasing delivery by a factor of >4 after seven hours.

The ability to control the drug delivery profile from a passive transdermal patch depends on the drug, the patch design, and the formulation. Transdermal delivery profiles are characterized by a period of onset, followed by a period of sustained delivery (from hours up to a week), followed by a period of decreasing delivery. The period of onset is typically longer than that for oral delivery, and may take, at a minimum, several hours to achieve Cmax, and can be controlled by the patch design and chemical permeation enhancers used in the formulation. The period of decreasing delivery can be controlled by the formulation design, or by removing the patch.

This study addresses the feasibility of passively delivering a drug from a transdermal patch based on requirements specified for flumazenil and thereby provides evaluation of drug diffusion across the skin at a feasible rate. The paper assessment does not consider feasibility associated with pharmacokinetic parameters or clinical efficacy. The assessment involves first qualitatively assessing the physicochemical properties and target transdermal dose of the drug by comparing them to the properties of an ideal passive transdermal drug.

Compounds that are similar to the ideal transdermal drug may provide good transdermal drug candidates. Then, the target transdermal patch product requirements, namely the target dose and patch size, are quantitatively evaluated for feasibility by comparing the target dose, translated into a target permeability coefficient, to the maximum permeability coefficient for drugs that are delivered passively from transdermal patches, based on those that have been commercialized into transdermal patch products. This maximum limit is defined empirically by a 3M model, which can be used to estimate the largest flux and minimum patch size of the proposed drug product. Drugs that have patch size estimates less than or equal to the target patch size are feasible drugs for transdermal drug delivery. In addition, it is feasible to utilize 3M Drug Delivery Systems proprietary drug-in-adhesive technology to develop transdermal patches for these drugs.

Transdermal drug delivery involves passive diffusion of a drug across the skin. The stratum corneum layer, a heterogeneous, tightly packed layer of dead skin cells, is considered the rate limiting barrier in the skin. Ideal transdermal drugs, defined as those that diffuse easily across this layer, tend to be small, unionized, lipophilic molecules. Drugs with low melting points (e.g. oils) tend to diffuse easily across the skin.

The target transdermal flux is calculated by dividing the target transdermal dose by the patch area. Using Fick's Law of Diffusion, the steady-state transdermal flux (μg/cm²/hr) of a drug across the stratum corneum, j, is described by Equation 1:

J=D/1Δc=Pc_{oct}

where D is the diffusion coefficient of the drug in the stratum corneum (cm²/hr), 1 is the thickness of the stratum corneum, P is the permeability coefficient (cm/hr), and Δc is the drug's concentration gradient across the stratum corneum (μg/mL), which is commonly approximated in the literature, under infinite sink conditions, by the drug's solubility in octanol (coct).

The target drug flux is estimated by dividing the target daily transdermal dose (mg/day) by the target patch size (cm²). If the target patch size is not defined, then a value of 40 cm² is used. Then, using equation (1), the target permeability coefficient can be estimated by dividing the target drug flux by the drug's solubility in octanol.

### 3M Drug Delivery Systems Proprietary Transdermal Drug-in-Adhesive Technology

a. Patch Design:
(i) 3M drug-in-adhesive technology utilizes pressure sensitive adhesives and excipients, if needed, for permeation enhancement, drug solubility, or formulation stability.
(ii) patch area is modified based on the dosage
(iii) Wear duration is customized as required and wear periods vary from several hours through one day to multi-day, up to one week, where varying wear periods can be accommodated by changing the patch's drug loading.
(iv) The aim is to provide smooth plasma profile and maintain drug serum levels within specified target minimum/peak levels.

### Flumazenil Transdermal Paper Feasibility

Flumazenil is a neutral, low molecular weight, lipophilic molecule, properties which makes it attractive for passive transdermal delivery. Although it has a high melting point, several drugs in commercial transdermal products have similar melting points.

TABLE 20A
Flumazenil for Transdermal Delivery Mode

| | Ideal | | |
|---|---|---|---|
| | Transdermal | | |
| Property | Drug | Flumazenil | vs. Ideal |
| Molecular Weight | ≦500 | 303 | + |
| (g/mole) | | | |
| Ionization | Neutral | Neutral | + |
| Melting Point (° C.) | ≦150 | 198 | − |
| log P | 1 to 3 | 1 | + |
| Transdermal Dose | ≦10 mg/day | 0.6 mg/hr for 2 | − 0 − for 1 day |
| (low dose) | for 1 day patch | hours (equiv. to | − for 3, 4 day |
| | ≦4 mg/day for | 14.4 mg/day) | − for 7 day |
| | 3, 4 day patch | | |
| | ≦1 mg/day | | |
| | for 7 day patch | | |

Key: + (in the ideal range), −0− (borderline ideal), − (outside the ideal range)

TABLE 20B
Passive Transdermal Feasibility Estimates
using the 3M Empirical Model

| | Target | Estimate from 3M Model |
|---|---|---|
| Transdermal Permeability | 2.94 × 10⁻⁴ | 2.57 × 10⁻⁴ |
| Coefficient (cm/hr): | | |
| Passive Transdermal Flux | 15.0 | 13.1 |
| (μg/cm²/hr) | | |
| Patch Area (cm²): | ≦40 | 46 |

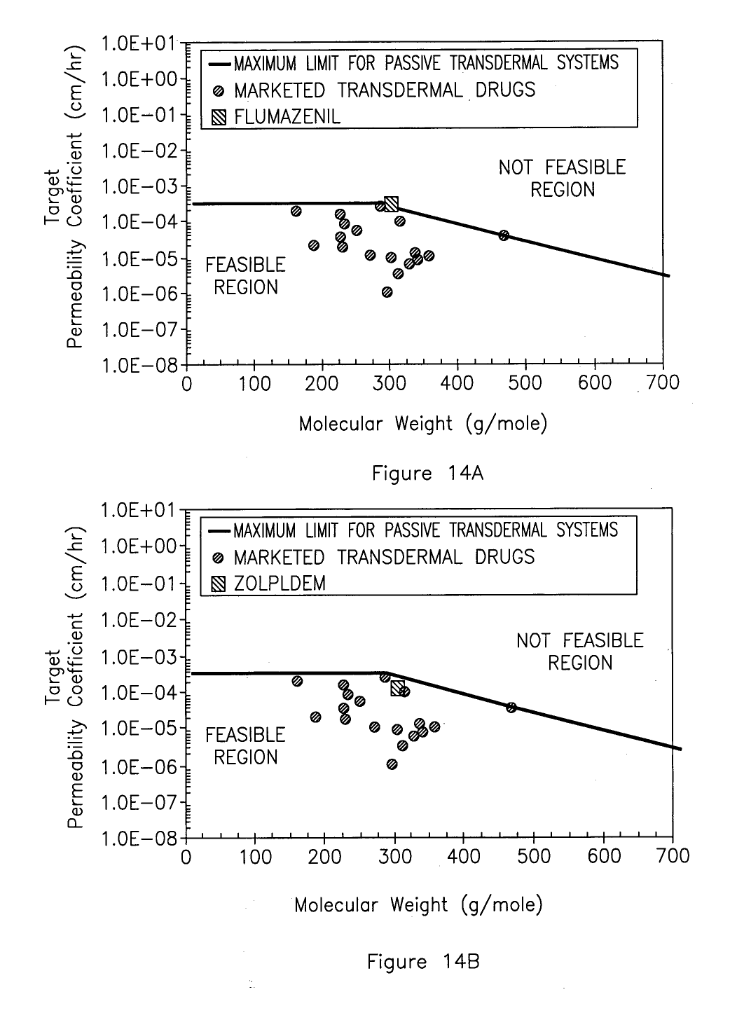

The permeability coefficient measured for flumazenil is shown in FIG. 14A. The high target dose is borderline feasible for the specified dosing and delivery duration requirements. Overall, transdermal delivery of flumazenil is borderline for the target product definition.

### Example 19

### Passive Transdermal Delivery of Zolpidem with 3M Drug Delivery Drug-in-Adhesive Technology

Zolpidem is a neutral, low molecular weight, lipophilic molecule, properties which are attractive for passive transdermal delivery. Although it has a high melting point, several drugs in commercial transdermal products have similar melting points.

TABLE 21
Comparison of Zolpidem to an Ideal Passive Transdermal Drug.

| | Ideal | | |
|---|---|---|---|
| | Transdermal | | |
| Property | Drug | Flumazenil | vs. Ideal |
| Molecular Weight | ≦500 | 307 | + |
| (g/mole) | | | |
| Ionization | Neutral | Neutral | + |
| Melting Point (° C.) | ≦150 | 196 | − |
| log P | 1 to 3 | 3.2 | + |
| Transdermal Dose | ≦10 mg/day | ~0.3 mg/hr for 7 | + for 1 day |
| (low dose) | for 1 day patch | hours (equiv. to 7 | − for 3, 4 day |
| | ≦4 mg/day for | mg/day) | − for 7 day |
| | 3, 4 day patch | | |
| | ≦1 mg/day | | |
| | for 7 day patch | | |

Key: + (in the ideal range), −0− (borderline ideal), − (outside the ideal range)

TABLE 22 A
Passive Transdermal Feasibility Estimates
using the 3M Empirical Model

| | | Estimate from |
|---|---|---|
| | Target | 3M Model |
| Transdermal Permeability | 1.30 × 10⁴ | 2.40 × 10⁴ |
| Coefficient (cm/hr): | | |
| Passive Transdermal Flux (μg/cm²/hr) | 7.3 | 13.5 |
| Patch Area (cm²): | ≦40 | 22 |

The permeability coefficient measured for zolpidem is shown in FIG. 14B. While the target transdermal dose of 0.3 mg/hr is a feasible dose, the delivery profile requirements, particularly the short duration for delivery onset, is borderline feasible.

### Example 20

### Iontophoretic Delivery of Flumazenil—Part I

In this study the potential of iontophoresis to deliver flumazenil through the skin is determined. Iontophoresis involves the application of a weak, direct electric current to a membrane (typically the skin) so as to facilitate the passage of a poorly-absorbed active agent. The approach is usually applied to ionic species, which have negligible passive permeability. A cation (a positively-charged ion) is therefore formulated at the anode (the positive electrode) and, upon application of the field, is driven across the skin by electromigration, i.e., the electrorepulsion between the ion and electrode of like charge. A second mechanism of transport usually referred to as electro-osmosis or convective solvent flow has its origin on the skin permselective properties. The skin has a net negative charge at physiological pH, thus being permselective to cations, a convective solvent flow occurs on the same direction of transport of the counterions, therefore in the anode to cathode direction.

Flumazenil (MW. 303.29) is a weak base (pKa=1.78 product information to 1.14±0.2 SciFinderScholar) and is sparingly soluble. As detailed above, the common IV dose for flumazenil is 0.2 mg administered over 15 seconds; it may be repeated to a maximum 1 mg and the mean dose required for efficacy is 2.7 mg or within the range of 1-3 mg. Accordingly, the parameters for effective iontophoresis are:
(a) a flux of 660 nmoles (0.2 mg, MW=303.29 daltons) delivered in 15 seconds or 158 μmoles/h.
(b) for a maximum 3 mg with administration time of 5-10 minutes; the target flux is 9.89 moles in 5-10 minutes or 118-60 μmoles/h.

The following challenges for formulating flumazenil in an iontophoretic device were identified:
1. Based on the aforementioned information a pH<2 would be required to ensure 50% ionization. Apparently, it would seem interesting to formulate a low pH donor to increase flumazenil ionization (activate the electrorepulsion mechanism), however, this strategy is unadvisable to two reasons: (i) this low pH would result in skin irritation and (ii) a decreased drug transport as result of competition for charge carrying with the very mobile. [H⁺](10 mM at pH 2) and (iii) a decreased or even partially reversal of the skin cation permselectivity which would result in decreased drug transport as the electroosmotic contribution and the cation transport numbers (electrorepulsion) are decreased at low pH.
2. If the drug is delivered uncharged (see point 1) the only mechanism available for drug transport is electro-osmosis. The strategy to maximize transport involves in this case: maximizing drug concentration in the vehicle and the convective solvent flow in the anode-to-cathode direction (achieved at pH 6-8 and low ionic strength).
3. Flumazenil has a relatively low solubility (about maximum 33 mM solubility in pure ethanol and ˜23 mM in Propylene glycol, PEG 300 and PEG 400).
4. While the dose (mass) of flumazenil required is deliverable via iontophoresis the rate of delivery (mass/time) required for a practical application may not be simply achievable.

The iontophoretic delivery of flumazenil from simple aqueous and cosolvent formulations across dermatomed pig skin is measured and compared to the target fluxes above determined. All experiments involve at least 5 replicates.

First the optimization of the formulation is addressed. The apparent solubility of flumazenil in cosolvent systems (ethanol:water, PEG:water or PEG:water) and other aqueous systems containing surfactants is determined.

Second, the first group of experiments uses donor phase containing the drug in the optimized donor system. The receptor phase for all experiments is pH 7.4 buffered normal saline. A different receptor phase can be used to ensure good solubilization of any permeated drug.

In a preliminary experiment 0.4 mA direct constant current is applied for 6 hours from Ag/AgCl electrodes, prepared as commonly known. Samples are taken every 1 hour from the receptor solution and analyzed by HPLC. Upon completion of the experiment, the stratum corneum is sampled by tape-stripping; the drug is extracted with an appropriate solvent and analyzed by HPLC.

The next experiments use a donor phase containing drug optimized donor systems. Commonly, the same receptor phase is used. Sampling times and length of experiments are deduced from the preliminary experiment.

These experiments explore the effect of donor solution. Initially, the iontophoretic transport of flumazenil delivered from various vehicle formulations is studied. Optionally, a marker of electroosmosis, such as paracetamol, is included, in order to evaluate the magnitude of the driving force for the drug. In addition, the effect of ionic strength/pH/polarity is studied, as follows:
(i) Anodal delivery: Ionic strength is kept as low as possible, and pH in the range 7-8 to increase electroosmosis in the anode-to-cathode direction. A source of chloride ions must be available at the anode for the electrochemical reaction, which contributes a certain ionic strength. Whether a vehicles is oxidized at the anode is tested a priori.
(ii) Cathodal delivery: Ionic strength should is as low as possible, and pH ˜4 (lower than the skin isoelectric point) to increase electroosmosis in the cathode-to-anode direction. No source of chloride ions is required, just some conductivity. However, given that the subdermal solution is kept at 7.4 (physiological range) the permselectivity of the skin may not completely reverse.
(iii) Effect of current: The current is initially set at 0.5 mA. The possible effect of current density is determined for the best formulation.
(iv) Passive controls is carried out for the best formulation.

### Example 21

### Iontophoretic Delivery of Flumazenil—Part II

HPLC analysis was performed under the following parameters: (i) HPLC Reverse Phase Assay; (ii) Mobile Phase: water:Acetonitrile: Acetic Acid=80:20:0.1; (iii) Flow rate: 1 ml/min; (iv) UV Detector Setting: 247 nm; (v) Pressure: 100 bar; (vi) Injection volume: 20 ul; (vii) Retention time: 4.5 min; and (ix) Run time: 10 min.

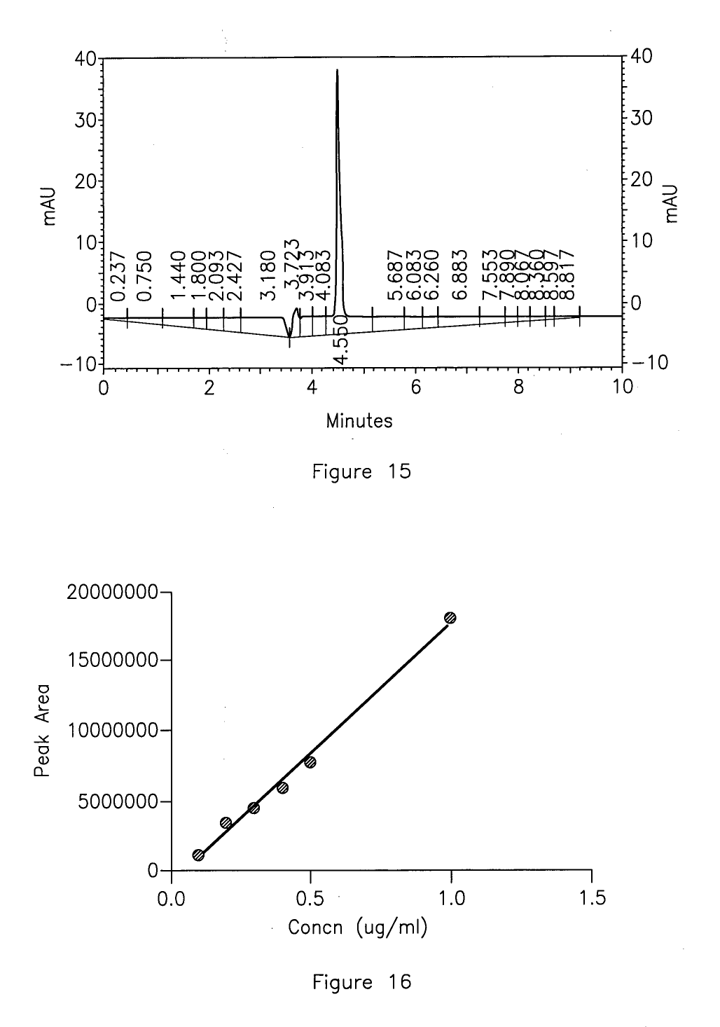

The resulting chromatorgram (FIG. 15) indicates a peak at 4.5 min. The correlation between flumazenil concentration and peak area was also determined (FIG. 16) using the following solution Standards
(i) 0.1 ug/ml flumazenil in mobile phase (Water:Acetonitrile: Acetic Acid=80:20:0.1)
(ii) 0.2 ug/ml flumazenil in mobile phase (Water:Acetonitrile: Acetic Acid=80:20:0.1)
(iii) 0.3 ug/ml flumazenil in mobile phase (Water:Acetonitrile: Acetic Acid=80:20:0.1)
(iv) 0.4 ug/ml flumazenil in mobile phase (Water:Acetonitrile: Acetic Acid=80:20:0.1)
(v) 0.5 ug/ml flumazenil in mobile phase (Water:Acetonitrile: Acetic Acid=80:20:0.1)
(vi) 1.0 ug/ml flumazenil in mobile phase (Water:Acetonitrile: Acetic Acid=80:20:0.1)

The graph (FIG. 16) presenting the correlation between peak area and concentration indicates a highly liner correlation with R²=0.9924.

### Gel Electrophoresis

The following parameters were applied in order to evaluate migration in agarose gel (1%; 3 mm thick):

Total Width: 15.5 cm; Length: 15 cm; No of Lanes: 4; No of sections: 10; Buffer solution used: TAE (Tris borate, Acetic acid, EDTA); Well vol: 10 ul/well; Voltage used: 50V-100V DC; and Run Time: 1 hr.

The following solutions were tested:

1. 0.66 mg/ml flumazenil pH 4.5—Positive Electrode
2. 0.66 mg/ml flumazenil pH 5.5—Positive Electrode
3. 0.66 mg/ml flumazenil in tween80 pH 7.2—Positive Electrode

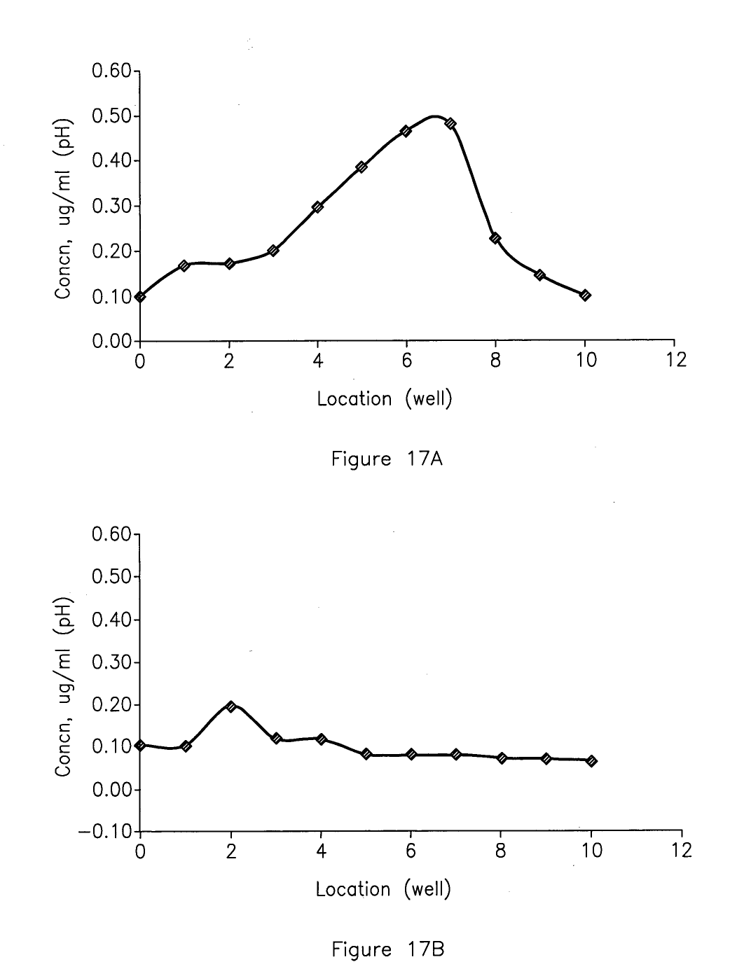

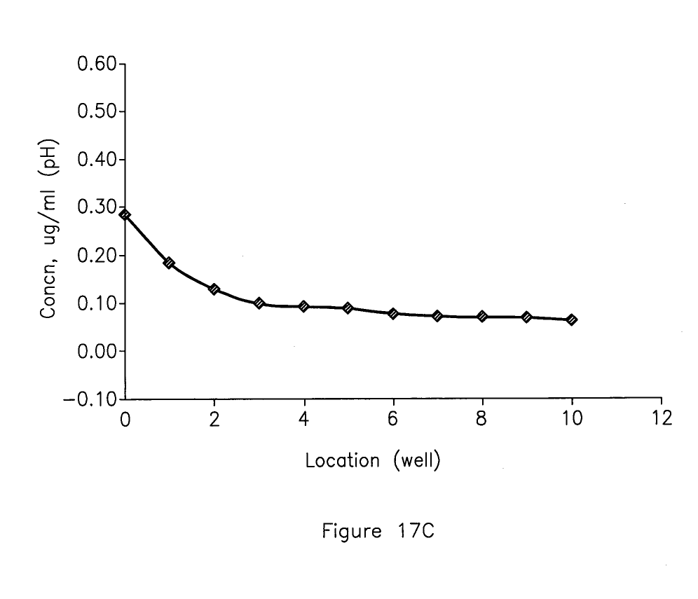

The results indicate that at pH 4.5 the migration of flumazenil in agarose gel is most efficient (FIG. 17A-C).

Analytical testing with HPLC on a skin sample (Franz cells) was performed between 0 to 0.1 μg/ml (N=6) with zero (0) current (passive), 0.2 mA and 0.5 mA using flumazenil formulation at pH 4.5, resulted with a standard curve of a straight line (r²=0.9958; B=2263637.838; A=143498.027) suggesting good correlation. Representative data collected for obtaining the standard curve are provided in Table 22B.

TABLE 22B
Standard curve.

| Sample No | Concentration (μ/ml) | Peak Area |
|---|---|---|
| 1 | 0.01 | 170917 |
| 2 | 0.04 | 225674 |
| 3 | 0.08 | 328176 |

### Example 22

### Formulations Containing Surfactants

Formulations containing Benzalkonium chloride (BZC), a cationic surfactant and solubilizing agent (by increasing the CMC—critical micellar concentration), were prepared in a final volume of 5 g with the following excipients:
Citric buffer 10 mM pH 4.0—prepared according to the following composition: Citric acid anhydrous—0.1199% w/v and Sodium Citrate dihydrate—0.1105% w/v dissolved in water and pH adjusted to 4.0±0.2, if needed.
HPCD 30% w/w in Citric buffer 10 mM pH 4.0 solution.
Menthol in EtOH solution was prepared by dissolving Menthol in Ethanol absolute in a ratio of 1/1 w/w.
Benzalkonium chloride in water solution was prepared by dissolving the cationic surfactant in water.
The alkyltrimethyl ammonium chloride or bromide (where the no. of alkyl carbons is 16 or 18) in water solution is prepared by dissolving the cationic surfactant in water.

For all formulations the required amount of flumazenil was weighed in a glass beaker and other materials were added according to Tables 23-25, respectively. The mixture were then stirred using a magnetic stirrer plate until a clear solution was obtained.

TABLE 23
Formulation optimization matrix without HPCD (% w/w)

| Material name | F22-3 | F22-4 | F22-5 |
|---|---|---|---|
| Flumazenil | 1 | 1 | 1 |
| Ethanol | 25 | 25 | 25 |
| Propylene glycol | 25 | 25 | 25 |
| Citric buffer 10 mM pH 4.0 | 47.4 | 44.4 | 48.7 |
| Menthol in EtOH (1/1 w/w) | 0.1 | 0.1 | 0.1 |
| Benzalkonium chloride 50% solution | | | 0.2 |

TABLE 24
Formulation optimization matrix with HPCD (% w/w)

| Material name | F26-5 |
|---|---|
| Flumazenil | 1 |
| Ethanol | 20 |
| Propylene glycol | 20 |
| 30% HPCD in citric buffer 10 mM pH 4.0 | 58.7 |
| Menthol in EtOH (1/1 w/w) | 0.1 |
| Benzalkonium chloride 50% solution | 0.2 |

### Example 23

### Formulations Containing Increased Menthol Concentration

TABLE 25
Flumazenil 0.4% formulation containing menthol 0.2% (% w/w)

| Material name | F26-6 |
|---|---|
| Flumazenil | 0.4 |
| Ethanol | 20 |
| Propylene glycol | 20 |
| 30% HPCD in citric buffer 10 mM pH 4.0 | 59 |
| Menthol in EtOH (1/1 w/w) | 0.4 |

### Example 24

### Appearance of Formulations After Storage

TABLE 26
Appearance of Flumazenil formulations following storage.

| | 4 mg/ml | | | | | | 2 mg/ml | | | | | | | | |
|---|---|---|---|---|---|---|---|---|---|---|---|---|---|---|---|
| Formulation name | F20 | F21 | F22 | F24 | F25 | F26 | F27 | F28 | F29 | F30 | F31 | F32 | F33 | F34 | F16 |
| Ethanol (% w/w) | 30 | 25 | 25 | 30 | 25 | 20 | 10 | 20 | 25 | 25 | 5 | 10 | 20 | 25 | 10 |
| Propylene glycol (% w/w) | 30 | 25 | 25 | 30 | 25 | 20 | 10 | 20 | 25 | 25 | 5 | 10 | 20 | 25 | 40 |
| 30% HPCD in citric | | | | 40 | 50 | 60 | | | | | 90 | 80 | 60 | 50 | |
| buffer 10 mM pH 4.0 | | | | [12] | [15] | [18] | | | | | [27] | [24] | [18] | [15] | |
| (% w/w of HPCD | | | | | | | | | | | | | | | |
| solution and [% w/w of | | | | | | | | | | | | | | | |
| total HPCD]) | | | | | | | | | | | | | | | |
| Citric buffer 10 mM pH | | | 50 | | | | | | | 50 | | | | | |
| 4.0 | | | | | | | | | | | | | | | |
| Water (% w/w) | 40 | 50 | | | | | 80 | 60 | 50 | | | | | | 50 |
| Appearance | c | c | c | c | c | c | n/c | c | c | c | n/c | n/c | c | c | c |
| pH after preparation | 5.80 | 6.14 | 4.93 | 5.56 | 5.35 | 5.13 | | 7.38 | 7.03 | 5.03 | | | 5.22 | 5.44 | 5.48 |
| pH after 1 week storage | 6.78 | 6.98 | 5.07 | 5.05 | 5.35 | 5.14 | | 5.93 | 6.48 | 4.99 | | | 5.22 | 5.35 | 7.29 |
| at 25° C. | | | | | | | | | | | | | | | |
| pH after 1 week storage | 6.77 | | 5.04 | | | 5.22 | | | | | | | 5.24 | | |
| at ambient temperature | | | | | | | | | | | | | | | |

NA—not applicable; c—clear; n/c—not clear

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-59. (canceled)

60. A liquid formulation comprising flumazenil as an active ingredient, a solubilizing agent selected from an alcohol, a glycol and a combination thereof, a buffering agent, and at least one agent selected from the group consisting of: a penetration enhancer, a surfactant and cyclodextrin.

61. The formulation of claim 60, wherein the cyclodextrin is hydroxypropyl 3-cyclodextrin (HPCD) having a pH of about 4 and a concentration in acidic buffer of about 30%.

62. The formulation of claim 60, comprising a preservative selected from the group consisting of: benzyl alcohol, propylparaben, methylparaben and combinations thereof.

63. The formulation of claim 60, comprising the penetration enhancer menthol.

64. The formulation of claim 60, wherein the buffering agent is selected from the group consisting of: citric buffer, sodium chloride and combination thereof.

65. The formulation of claim 60, comprising the surfactant benzalkonium chloride.

66. The formulation of claim 60, comprising a plurality of agents selected from the group consisting of: a penetration enhancer, a surfactant, a complexation agent, a solubilizing agent and a preservative.

67. The formulation of claim 60, in the form of a sublingual spray, wherein the concentration of flumazenil is at least 0.2% w/w.

68. The formulation of claim 60, wherein the concentration of flumazenil is within the range of 0.2% w/w to 2% w/w.

69. A method of treating excessive sleepiness during waking hours in a subject treated with a sleep drug, the method comprising administering to a subject in need thereof an effective amount of the formulation according to claim 60.

70. The method of claim 69, wherein the formulation is administered in a route selected from the group consisting of: repetitive administration, self-administeration, sublingual administration, subdermal administration, transdermal administration, inhalation and transmucosal administration.

71. The method of claim 69, wherein the formulation is administered in an amount within the range of about 0.2 mg to about 2.0 mg flumazenil per dose.

72. The method of claim 69 wherein said formulation is administered at least about 2 hours after the administration of the sleep drug, concurrently with the sleep drug or upon awakening from a sleep drug-induced sleep period.

73. A method for treating or preventing breathing compensation associated with general anesthesia or for treating alcohol intoxication, comprising administering to a subject in need thereof, an effective amount of the formulation according to claim 60.

74. The method of claim 73 treating or preventing breathing compensation, wherein the subject is suffering from sleep apnea or has been diagnosed with a form of insomnia selected from the group consisting of sleep onset insomnia; sleep maintenance insomnia; end of sleep insomnia; idiopathic hypersomnia; idiopathic insomnia transient insomnia; subacute insomnia; chronic insomnia; narcolepsy; Time Zone Change Syndrome, and a combination thereof.

75. The method of claim 73 for treating or preventing breathing compensation wherein said formulation is administered at least about 2 hours after the administration of the sleep drug or upon awakening from a sleep drug-induced sleep period.

76. The method of claim 73, comprising self-administering of said formulation.

77. The method of claim 73 for treating alcohol intoxication, comprising a treatment selected from the group consisting of: reversing the effects of alcohol intoxication, reducing the effects of alcohol intoxication, alleviating the effects of alcohol intoxication, alcohol withdrawal and improving performance after alcohol consumption.

78. The method of claim 77, wherein the formulation is administered in an amount within the range of about 0.2 to about 10 mg flumazenil, over a 24 hour period.

79. The method of claim 77, wherein the formulation is administered in an amount within the range of about 0.2 to about 2.0 mg flumazenil per dose.
